(19) United States
(12) Patent Application Publication
(10) Pub. No.: US 2022/0125876 A1
(43) Pub. Date: Apr. 28, 2022

(54) A METHOD OF IMMUNE MODULATION BY MODULATING ABCF1

(71) Applicant: Pacific Myco Biosciences, Ltd., Kelowna (CA)

(72) Inventors: Wilfred JEFFERIES, Surrey (CA); Hitesh ARORA, Surrey (CA); Sara WILCOX, Surrey (CA); Lonna MUNRO, Surrey (CA); Cheryl PFEIFER, Surrey (CA)

(73) Assignee: Pacific Myco Biosciences, Ltd., Kelowna, BC (CA)

(21) Appl. No.: 17/430,552

(22) PCT Filed: Feb. 14, 2020
(86) PCT No.: PCT/CA20/50192

§ 371 Date: Aug. 12, 2021

Related U.S. Application Data
(60) Provisional application No. 62/805,397, filed on Feb. 14, 2019.

(51) Int. Cl. A61K 38/17; A61P 19/02; A61P 37/02

(52) U.S. Cl. CPC A61K 38/177; A61P 37/02; A61P 19/02

(57) ABSTRACT

The present invention relates to methods of immune modulation. In particular, the present invention relates to regulation of inflammation and immune responses by modulation of ABCF1. Modulation of ABCF1 may be useful in the treatment of sepsis, autoimmune diseases, cancer or infections.

### FIELD OF THE INVENTION

The present invention relates to methods of immune modulation. In particular, the present invention relates to regulation of inflammation and immune responses by modulation of ABCF1.

### BACKGROUND

Inflammation and immune responses are tightly controlled cellular mechanisms that help maintain cellular homeostasis. These mechanisms are governed by several proteins that regulate a cascade of downstream effectors. Modulation of these downstream effectors may be used to treat disease, including treatment of sepsis, inflammatory bowel disease, Crohn's disease and cancer. ABCF1 is an ABC transporter family protein that has been shown to regulate the innate immune response and is a risk gene for autoimmune pancreatitis and arthritis. Unlike other ABC family members, ABCF1 lacks the transmembrane domain and does not appear to function as a transporter. The ABCF1 gene is located in the class I region of the major histocompatibility complex locus on chromosome 6 in humans and on chromosome 17 in mice. Previous studies have shown that ABCF1 participates in translation initiation through its association with eIF2 and ribosomes (Garcia-Barrio, M., et al., 2000; Marton, M. J., et al., 1997; Paytubi, S., et al., 2008; Campbell, S. G., et al., 2005; Pestova, T. V. et al., 2006). ABCF1 is known to be located in the cytoplasm and nucleoplasm, but not in the nucleolus (Paytubi, S., et al., 2008). Gene expression of ABCF1 has been shown to be elevated substantially in human synoviocytes isolated from the inflamed joints of rheumatoid arthritis patients, and this increases further when stimulated with TNF-α (Richard, M., et al., 1998). Also, the ABCF1 locus is linked to increased susceptibility to autoimmune pancreatitis in the Japanese population (Ota, M. et al., 2007) and, importantly, ABCF1 has been associated with susceptibility to rheumatoid arthritis in European and Asian populations. Immunological studies in mouse embryonic fibroblasts have shown that ABCF1 associates with dsDNA and DNA sensing components HMGB1 and IFI204, and further interacts with SET complex members (SET, ANP32A and HMGB2) to facilitate cytosolic DNA sensing mechanisms.

ABCF1(+/−) mice appear normal under specific pathogen-free conditions. Studies of these mice showed that ABCF1 acts as a molecular switch between inflammatory pathways downstream of TLRs (Arora, H. et al., 2019). ABCF1 possesses an E2 ubiquitin enzyme activity, through which it controls the LPS—Toll-like Receptor-4 (TLR4)—mediated gram-negative insult by targeting key proteins for K63-polyubiquitination. K63-ubiquitination by ABCF1 shifts the inflammatory profile from an early phase MyD88-dependent to a late phase TRIF-dependent signalling pathway, thereby regulating TLR4 endocytosis and modulating macrophage polarization from M1 to M2 phase. Physiologically, ABCF1 regulates the shift from the inflammatory phase of sepsis to the endotoxin tolerance phase and modulates cytokine storm and interferon-β-dependent production by the immunotherapeutic mediator, SIRT1. Consequently, ABCF1 controls sepsis-induced mortality by repressing hypotension induced renal circulatory dysfunction. Further, ABCF1 is necessary to maintain macrophage polarization in M2b state and the lack of ABCF1 shifts the state to the pro-inflammatory M1 state (Arora, H. et al., 2019).

In the MyD88 pathway (M1 macrophage-like), the early phase of TLR4 signalling leads to UBC13 targeting TRAF6 for K63-polyubiquitination, which further targets cIAP1/2 for K63-polyubiquitination. cIAP1/2 then enhances K48-proteasomal degradation of ABCF1 and TRAF3. In the absence of ABCF1, TAK1 is phosphorylated, which leads to activation of MAPK and NF-kB pathways and elevated production of pro-inflammatory cytokines like TNFα, IL-1β, IL-6, thereby polarizing macrophages to M1 phenotype. Subsequently in the TRIF pathway (M2 macrophage-like), self K48-proteasomal degradation of cIAP1/2 results in K63-polyubiquitination of ABCF1 by TRAF6, which results in ABCF1 to bind and forms a complex with TRAF3 and SYK leading to the formation of K63-polyubiquitylated TRAF3 and SYK. This leads to TLR4 endocytosis into the endosomes, which then initiates TRIF-dependent TLR4 signalling and eventual production of IFN-I stimulated genes. This triggers phosphorylation of TBK1 that leads to phosphorylation and eventual dimerization of IRF3 and production of IFN-I stimulated genes. This shift from MyD88 to TRIF signalling by ABCF1 leads to increased production of IL-10, minimal production of TNFα, IL-1β, IL-6 and CD86, MHC-II surface markers and decreased CD206 levels, thus polarizing macrophages to M2b phenotype.

Immune response disfunction and inflammation are implicated in numerous diseases, there exists a need for modulating immune response and inflammation.

### SUMMARY OF THE INVENTION

An object of the present invention is to provide a method of immune modulation by modulating ABCF1.

In accordance with an aspect of the invention, there is provided a method of inhibiting an inflammatory response and/or an immune response in a patient in need thereof, the method comprising administering an agonist of ABCF1.

In accordance with another aspect of the invention, there is provided a method of inhibiting an inflammatory response and/or an immune response, the method comprising administering an ABCF1 protein or a polynucleotide encoding ABCF1. In some embodiments of the method of the invention, the ABCF1 protein is a soluble ABCF1 protein. In some embodiments of the method of the invention, the polynucleotide is a vector, optionally a viral vector.

In accordance with another aspect of the invention, there is provided a method of preventing and/or treating sepsis, the method comprising administering an agonist of ABCF1.

In accordance with another aspect of the invention, there is provided a method of preventing and/or treating sepsis, the method comprising administering an ABCF1 protein or a polynucleotide encoding ABCF1. In some embodiments of the method of the invention, the ABCF1 protein is a soluble ABCF1 protein. In some embodiments of the method of the invention, the polynucleotide is a vector, optionally a viral vector.

In accordance with another aspect of the invention, there is provided a method of treating an autoimmune disease, the method comprising administering an agonist of ABCF1.

In accordance with another aspect of the invention, there is provided a method of treating an autoimmune disease, the method comprising administering an ABCF1 protein or a polynucleotide encoding ABCF1. In some embodiments of the method of the invention, the ABCF1 protein is a soluble ABCF1 protein. In some embodiments of the method of the invention, the polynucleotide is a vector, optionally a viral vector.

In accordance with another aspect of the invention, there is provided a method of determining clinical outcome of diseases and/or disorders associated with increased or decreased inflammatory and/or immune responses, the method comprising determining expression of ABCF1.

In accordance with another aspect of the invention, there is provide use of an agonist of ABCF1 to reduce an inflammatory response and/or an immune response in a patient in need thereof.

In accordance with another aspect of the invention, there is provide a method of inhibiting an inflammatory response and/or an immune response, the method comprising administering an ABCF1 protein or a polynucleotide encoding ABCF1. In some embodiments of the method of the invention, the ABCF1 protein is a soluble ABCF1 protein. In some embodiments of the method of the invention, the polynucleotide is a vector, optionally a viral vector.

In accordance with another aspect of the invention, there is provided a method of preventing and/or treating sepsis, the method comprising administering an agonist of ABCF1.

In accordance with another aspect of the invention, there is provided a method of preventing and/or treating sepsis, the method comprising administering an ABCF1 protein or a polynucleotide encoding ABCF1. In some embodiments of the method of the invention, the ABCF1 protein is a soluble ABCF1 protein. In some embodiments of the method of the invention, the polynucleotide is a vector, optionally a viral vector.

In accordance with another aspect of the invention, there is provided a method of treating an autoimmune disease, the method comprising administering an agonist of ABCF1.

In accordance with another aspect of the invention, there is provided a method of treating an autoimmune disease, the method comprising administering an ABCF1 protein or a polynucleotide encoding ABCF1. In some embodiments of the method of the invention, the ABCF1 protein is a soluble ABCF1 protein. In some embodiments of the method of the invention, the polynucleotide is a vector, optionally a viral vector.

In accordance with another aspect of the invention, there is provided a method of determining clinical outcome of diseases and/or disorders associated with increased or decreased inflammatory and/or immune responses, the method comprising determining expression of ABCF1.

In accordance with another aspect of the invention, there is provided a method of modulating an immune response by modulating activity or expression of ABCF1.

In accordance with another aspect of the invention, there is provided a method of stimulating an immune response by inhibiting expression or activity of ABCF1. In some embodiments, the immune response is an anti-cancer or anti-pathogen immune response, optional the pathogen is a viral or bacterial pathogen.

In some embodiments of the invention, the methods are used to treat patients with autoimmune diseases including inflammatory bowel disease such as Crohn's disease or ulcerative colitis, rheumatoid arthritis, or pancreatitis.

### BRIEF DESCRIPTION OF THE FIGURES

These and other features of the invention will become more apparent in the following detailed description in which reference is made to the appended drawings.

FIG. 1 illustrates that ABCF1 negatively regulates pro-inflammatory cytokines in TLR2 and TLR9 signalling and positively regulates anti-inflammatory cytokines in TLR5 signalling. Briefly, bone marrow derived macrophages (BMDM) were treated with Abcf1 siRNA and stimulated for 24 hours with A) TLR2 agonist peptidoglycan (100 ng/ml), B) TLR9 agonist CpG DNA (5 μM), C) TLR5 agonist flagellin (100 ng/ml). Heat map representing fold change of various cytokines in the presence and absence of the agonists in cell culture supernatants is shown.

FIG. 2 illustrates that ABCF1 is necessary for ISG specific cytokine, transcription factor production and IRF3 dimerization after Poly I:C stimulation. A) BMDM overexpressing ABCF1 (Over Exp.) or BMDM treated with specific siRNA in the presence and absence of Poly I:C (10 μg/ml) for 24 hours were assessed. Heat map representing fold change of various cytokines and chemokines in cell culture supernatants are shown. B) Heat map representing fold change of various MAPK and ISG specific phosphoproteins and transcription factors from whole cell lysates (WCL) from Abcf1 siRNA treated and Poly I:C stimulated BMDM are shown. C) Cytoplasmic and nuclear fractions from BMDM treated with Abcf1 specific siRNA in presence and absence of Poly I:C were analyzed by immunoblot (IB) for levels of transcription factors involved in TLR3 signaling. D) WCLs were separated by native PAGE gel and were analyzed for IRF3 dimerization.

FIG. 3 illustrates that ABCF1 associates with OAS1a and controls 2′5′A activity of OAS1a. A) BMDM were treated with poly I:C (10 μg/ml) for 24 hours and WCL were immunoprecipitated with anti-ABCF1 antibody. The immunoprecipitates were immunoblotted with anti-ABCF1 and anti-OAS1a antibodies. B) ABCF1 was over expressed (Over Exp.) in BMDM, WCL were immunoprecipitated with anti-ABCF1 antibody and the immunoprecipitates were immunoblotted with anti-ABCF1 and anti-OAS1a antibodies. C) Protein levels of OAS1a, ABCF1 and RNaseL were analyzed from WCL from Abcf1 siRNA-treated BMDM in the presence or absence of Poly I:C. D) Increased pyrophosphate production (a readout of 2′5′A activity) was measured in scrambled and Abcf1 siRNA treated BMDM in the presence and absence of 2′5′A.

FIG. 4 provides a model for how ABCF1 regulates dsRNA infection via OAS1a, TLR3 and JAK-STAT signaling. Briefly, after dsRNA infection, several signaling cascades are triggered within the cell to eliminate the infection. One of the first antiviral proteins to be produced is OAS1a. ABCF1 binds to OAS1a in an ATP dependent manner and associates with dsRNA. This binding leads to production of 2′5′A, which binds and activates RNaseL, which degrades viral RNA. dsRNA can also be detected by TLR3 in the endosomes. Once activated TLR3 leads to phosphorylation and dimerization of IRF3 and shuttles it to the nucleus. IRF3 leads to production of IFNβ and other pro-inflammatory cytokines, a step regulated by ABCF1. IFNβ leads to production of positive feedback loop by activating JAK-STAT pathway. ABCF1 was found to be essential for STAT 2, 3 and 6 phosphorylation, which led to an even elevated levels of IFNβ hence helping in clearing viral infection.

FIG. 5 illustrates that ABCF1 positively regulates anti-inflammatory cytokines and ISG specific transcription factors and negatively regulates MAPK after LPS stimulation. A) ABCF1 was either over-expressed (Over Exp.) in BMDM or BMDM were treated with specific siRNAs in the presence and absence of LPS (100 ng/ml) for 24 hours. Heat map representing fold change of various cytokines and chemokines in cell culture supernatants. B) Heat map representing fold change of various MAPK and ISG specific phosphoproteins and transcription factors from Abcf1 siRNA and LPS treated BMDM WCL.

FIG. 6 illustrates that ABCF1 is necessary for FcgR II A mediated phagocytosis. A) Absorbance value of phagocytosis was measured either in Abcf1 siRNA treated BMDM incubated with fluorescent labeled IgG coated latex beads for 2 hours in presence and absence of LPS or Abcf1 siRNA treated BMDM incubated with fluorescent labeled E. coli for 2 hours. B) CD32 levels were analyzed by flow cytometry in treated BMDM. Bar graph represents change in mean fluorescence intensity (MFI) and error bars represent standard deviation. C) ABCF1 was overexpressed in BMDM in the presence and absence of LPS. WCL were immunoprecipitated with anti-CD32 antibody. The immunoprecipitates were immunoblotted with anti-CD32 and anti-phosphotyrosine antibodies. D) Heat map representing fold change in phosphorylation levels of SFK's in WCL from Abcf1 siRNA treated and LPS stimulated BMDM.

FIG. 7 provides a model for how ABCF1 regulates FcγR II A mediated phagocytosis. Briefly, ABCF1 was found to be essential for phosphorylation of ITAM's by SRC, LYN or FGR, which initiates FcγR mediated phagocytosis of E. coli particles. ABCF1 then targets SYK for K63-linked polyubiquitination, which is followed by SFK mediated phosphorylation of SYK. Phosphorylation of SYK leads to downstream phosphorylation of PLCγ2, FYN and other SFK's, which lead to actin polymerization and formation of the phagocytic cup. The downstream cascade leads to ABCF1 dependent elevated phosphorylation levels of STAT 2, 3 and 6, which leads to increase in production of anti-inflammatory cytokines.

FIG. 8 illustrates that ABCF1 polarizes macrophages to M2b state in TLR4 mediated LPS signaling. ABCF1 was either over-expressed (Over Exp.) or was knocked down with specific siRNA in BMDM in the presence and absence of LPS (100 ng/ml for 30 mins). A) Treated BMDM were stimulated with LPS and analyzed by flow cytometry for M1 (CD86 and MHC-II) and M2a (CD206) specific surface markers. Graphs represent change in mean fluorescence intensity (MFI) and error bars represent standard deviation. B) Heat map representing fold change of various cytokines and chemokines in cell culture supernatants. C) Whole cell lysates (WCL) were separated by native PAGE gel and were analyzed for IRF3 dimerization.

FIG. 9 illustrates that ABCF1 is necessary for macrophage polarization to M2b phenotype. ABCF1 was either over-expressed in BMDM or treated with specific siRNA in the presence and absence of LPS (100 ng/ml for 30 mins). A) Cytoplasmic and nuclear fractions, were analyzed by immunoblotted for levels of transcription factors involved in macrophage polarization. WCL from B) BMDM treated with Abcf1 specific siRNA in presence and absence of LPS and C) ABCF1 over expressed BMDM in the presence and absence of LPS, were immunoblotted with regulatory proteins, kinases and transcription factor.

FIG. 10 illustrates that ABCF1 negatively regulates MyD88-dependent signaling and pyroptosis and is necessary for TRIF-dependent signaling in BMDM. Briefly, Abcf1 siRNA treated BMDM were stimulated in the presence or absence of LPS (100 ng/ml) for 30 min and levels of phosphoproteins were measured from WCL. A) Heat map representing fold change of various MAPK and ISG specific phosphoproteins and transcription factors B) Heat map representing fold change of cell survival specific phosphoproteins.

FIG. 11 illustrates that ABCF1 is a novel class IV E2 Ubiquitin-conjugating enzyme and catalyzes the reaction with a cysteine residue at position 647. A) Murine ABCF1 protein sequence aligns with conserved regions of the UBC domain from other known murine E2 conjugating enzymes, using Clustal Omega software. B) ABCF1 has E2 Ubiquitin-conjugating activity. ABCF1 was immunoprecipitated from WCL's and was used in the in vitro ubiquitination assay with the addition of E1 activating enzyme and ubiquitin, in the presence or absence of the reducing agent DTT. ABCF1 appears to catalyze the conjugation of ubiquitin through the cysteine at position 647. C) According to gene structure, ABCF1 appears to be a class IV E2 conjugating enzyme.

FIG. 12 illustrates that ubiquitination activity of ABCF1 is necessary for TLR4 endocytosis. TLR4 endocytosis was analyzed by flow cytometry in BMDM A) Stimulated with various LPS concentrations for 30 mins. B) Stimulated with LPS at 10 ng/ml concentration for various time points. C) Treated BMDM stimulated with LPS (100 ng/ml) for 30 mins. D) Both WT and Abcf1+/− mice were intraperitoneally (i.p.) injected with 50 μg LPS, and after various time points, mice were euthanized and the spleens harvested. TLR4 surface expression on CD11b+ macrophages were measured using flow cytometry (n=9 mice per group). Graphs represent percentage change in MFI. Error bars represent standard deviation.

FIG. 13 illustrates that a change in the polyubiquitination state of ABCF1 enables TLR4 endocytosis as indicated by a shift from MyD88-dependent to TRIF-dependent signaling. A) BMDM were stimulated with LPS (100 ng/ml) at various time points, and WCL were then immunoprecipitated with anti-ABCF1 antibody and immunoblotted with anti-poly-ubiquitin antibodies. K63-polyubiquitination of ABCF1 represents activation of ABCF1, whereas K48-polyubiquitination of ABCF1 indicates proteasomal degradation. B) BMDM were stimulated with various concentrations of LPS (0-1000 ng/ml) for 30 min, and WCL were then immunoprecipitated with anti-ABCF1 antibody and immunoblotted with anti-polyubiquitin antibodies. C) cIAP1/2 K48-polyubiquitinates ABCF1. BMDM were treated with SMAC mimetic (SM) to degrade cIAP1/2, in the presence or absence of Dynasore (dynamin inhibitor) and were stimulated with LPS (100 ng/ml) for 30 min. WCL were then immunoprecipitated with anti-ABCF1 antibody and immunoblotted with anti-polyubiquitin antibodies. D) TNF-receptor-Associated Factor 6 (TRAF6) K63-polyubiquitinates ABCF1. BMDM were transfected with plasmids containing: Flag-tagged Traf6, HA-tagged WT ubiquitin, K48-ubiquitin, K63-ubiquitin and GFP-tagged Abcf1. WCL were blotted with respective antibodies to check the extend of over expression and were immunoprecipitated with anti-GFP antibody. The immunoprecipitates were analyzed by immunoblot with anti-HA antibody.

FIG. 14 illustrates that ABCF1 levels are negatively regulated by cIAP1/2 in the early phase of TLR4 signaling, whereas ABCF1 forms a complex with SYK, TRAF6 and TRAF3 and negatively regulates SYK and PLCγ2 phospho levels in late phase TLR4 signaling in BMDM. A) BMDM were treated with cIAP1/2 siRNA in presence and absence of LPS (100 ng/ml) for 30 mins and immunoblotted for anti-ABCF1, anti-TAK1, anti-phospho TAK1 and anti-cIAP1/2 antibodies. B) BMDM were treated with LPS (100 ng/ml) for 30 mins and WCL were immunoprecipitated with anti-ABCF1 antibody. The immunoprecipitates were immunoblotted with anti-ABCF1, anti-SYK, anti-TRAF6 and anti-TRAF3 antibodies. C) SYK was over expressed (Over Exp.) in BMDM, WCL were immunoprecipitated with anti-SYK antibody and the immunoprecipitates were immunoblotted with anti-ABCF1 and anti-SYK antibodies. D) TRAF3 was over expressed in BMDM, WCL were immunoprecipitated with anti-TRAF3 antibody and the immunoprecipitates were immunoblotted with anti-ABCF1 and anti-TRAF3 antibodies. E) Protein analysis of p-SYK and p-PLCg2 in Abcf1 siRNA-treated BMDM in presence or absence of LPS.

FIG. 15 illustrates that ABCF1 is a K63-specific E2 conjugating enzyme and targets SYK and TRAF3 for K63-polyubiquitination thereby regulating TLR4 early and late phase pathways. A) ABCF1 targets SYK for K63-polyubiquitination. BMDM were transfected with plasmids containing: Myc-tagged Syk, HA-tagged WT ubiquitin, K48-ubiquitin, K63-ubiquitin, GFP-tagged Abcf1WT, and/or Abcf1C647S. WCL were immunoprecipitated with anti-MYC and the immunoprecipitates were immunoblotted with anti-SYK and anti-HA antibodies. B) ABCF1 targets TRAF3 for K63-polyubiquitination. BMDM were transfected with Myc-tagged Traf3 and above-mentioned plasmids. WCL were immunoprecipitated with anti-MYC and the immunoprecipitates were immunoblotted with anti-TRAF3 and anti-HA antibodies. C) Binding of ABCF1-TRAF3 complex with TRIF is necessary for TRAF3 ubiquitination. BMDM were treated with Trif siRNA in presence or absence of LPS and WCL were immunoblotted with anti-TRIF, anti-p-TBK1 and anti-TBK1. WCL were immunoprecipitated with anti-ABCF1. The immunoprecipitates were immunoblotted with anti-TRAF3 and anti-ABCF1.

FIG. 16 provides a model for how ABCF1 regulates TLR4 endocytosis in macrophages. cIAP1/2 targets ABCF1 for K48-mediated proteasomal degradation, which inversely regulates early phase TLR4 mediated MyD88 signaling. Self K48-proteasomal degradation of cIAP1/2 triggers the onset of late phase TLR4 signaling. Degradation of cIAP1/2 results in activation and K63-polyubiquitination of ABCF1 by TRAF6. ABCF1 then forms a complex with TRAF6 and SYK and targets SYK for K63-polyubiquitination in a RING domain like fashion. This leads to SYK and subsequent PLCg2 phosphorylation, which leads to TLR4 endocytosis and activation of TRIF dependent signaling. ABCF1 also forms a complex with TRAF3 and TRIF and targets TRAF3 for TRIF dependent K63-polyubiquitination in a RING domain like fashion, thereby modulating IRF3 phosphorylation and dimerization hence polarizing macrophages to M2b phenotype.

FIG. 17 illustrates that ABCF1 is necessary for TRIF-dependent transition from SIRS to ET phase during sepsis. A) Abcf1Het-ETT mice produce more pro-inflammatory cytokines and revert to SIRS phase. Serum was isolated from whole blood from both WT and Abcf1+/− mice, which were subjected to either NETT or ETT treatments. Heat map represents fold change of cytokines and chemokines from mouse serum. B) Abcf1+/− mice die earlier than WT mice when treated with either NETT or ETT treatments. Percentage survival after second LPS injection with n=9 mice per group was checked until 96 hours, p value <0.001. C) BMDM WCL from NETT or ETT mice were analyzed for the expression of regulatory proteins, phosphoproteins and caspases. D) ABCF1 is necessary for K63-polyubiquitination of TRAF3 in ET phase. BMDM WCL were immunoblotted with anti-TRAF3 and later immunoprecipitated with anti-TRAF3. The immunoprecipitates were immunoblotted with anti-TRAF3 and polyubiquitin antibodies. E) BMDM WCL were separated by native PAGE gel and were analyzed for IRF3 dimerization. All cytokine and protein analysis were done after 3 hours of second LPS injection.

FIG. 18 illustrates that ABCF1 is necessary for TRIF dependent transition from SIRS to ET phase and apoptosis during sepsis. Analysis of phosphoprotein levels in BMDM. BMDM were isolated from NETT or ETT mice and WCL were prepared. A) Heat map representing fold change of MAPK and ISG specific phosphoproteins and transcription factors levels in BMDM. B) Heat map representing fold change of phospho levels of cell survival proteins and various Src family kinases. C) WCL's from Abcf1 siRNA treated and LPS stimulated BMDM were analyzed by immunoblotted for levels of proteins specific for pyroptosis.

FIG. 19 illustrates that ETT-Abcf1+/− mice revert back to SIRS phase of sepsis and die due to Renal Circulatory Failure. ETT-WT and ETT-Abcf1+/− mice were perfused and sacrificed after 3 hours of second LPS injection. Kidney tissues were fixed in 4% paraformaldehyde for 24 hours, washed in PBS and imbedded in paraffin. 5-μm-thick sections were cut and were stained with hematoxylin and eosin (HE). The slides were seen under Olympus BX51 microscope and images were taken with Olympus cellSens software. Black arrows represent areas of red blood cells sludging.

FIG. 20 shows that ABCF1 polarizes macrophages to the M2 state in response to LPS stimulation. ABCF1 was silenced with siRNA in BMDMs in the presence or absence of LPS (100 ng/ml-30 mins). A) Treated BMDMs were stimulated with LPS and analyzed. Bar graphs represent mean fluorescence intensity (MFI) and error bars represent mean±standard deviation and p-values are indicated. B) Heatmap representing fold change of various cytokines and chemokines in BMDM cell culture supernatants, p-value<0.01. C) Whole cell lysates (WCLs) were separated by native PAGE gel and were analyzed for IRF3 dimerization. D) BMDMs were treated with Cobalt Chloride CoCl₂ (100 μM for 24 hours) and WCLs were prepared and immunoblotted (IB). E) WCLs from BMDMs treated with Abcf1-specific siRNA in the presence or absence of LPS and were immunoblotted.

FIG. 21 illustrates that ABCF1 is a class IV E2 ubiquitin-conjugating enzyme whose activity is critically dependent on a cysteine residue at position 647. A) The protein sequence of the UBC domain of murine ABCF1 was aligned with UBC domain sequences of known murine E2 conjugating enzymes. B) Recombinant proteins were incubated in the presence or absence of cofactors and DTT in an in vitro ubiquitination assay and reactions were immunoblotted. C) The in vitro ubiquitination assay described in (B) was performed using ABCF1 immunoprecipitated from WCLs of BMDMs transfected with either Abcf1 WT or Abcf1 C647S overexpression constructs. Endogenous ABCF1 expression was silenced using specific siRNA before transfection of Abcf1 C647S. The reactions were analyzed as in B). D) The in vitro ubiquitination assay described in (C) was performed using ABCF1 immunoprecipitated from WCLs of BMDMs and reactions were immunoblotted with ABCF1 antibody conjugated with FL4 and ubiquitin antibody conjugated with FL1. E) Various classes of E2 enzymes are shown w.r.t. N- and C-terminus overhangs.

FIG. 22 illustrates a change in the poly-ubiquitination state of ABCF1 is associated with TLR4 endocytosis. Abcf1 siRNA-treated BMDMs were stimulated in the presence or absence of LPS (100 ng/ml for 30 min) and expression of phospho-proteins in WCLs was measured. A) Heatmap representing fold change of various MAPK and ISG phospho-proteins and transcription factors, p-value<0.01. B) Heatmap representing fold change of phospho-proteins associated with cell survival, p-value<0.01. C) BMDMs were stimulated with LPS for the indicated time intervals, cells were then lysed and WCLs were immunoprecipitated with ABCF1 antibody, followed by immunoblotted. D) BMDMs were stimulated with various concentrations of LPS for 30 min, after which the presence of polyubiquitinated species of ABCF1 was determined as in (C).

FIG. 23 illustrates that the poly-ubiquitination state of ABCF1 is regulated by cIAP1/2 and TRAF6. A) BMDMs were treated with SMAC mimetic (100 nM) and/or Dynasore (80 μM) in the presence or absence of LPS. Cells were then analyzed for the presence of polyubiquitinated species of ABCF1 and WCLs were IP with ABCF1 antibody, followed by immunoblotted. B) BMDMs were co-transfected with the indicated combinations of the constructs. Cells were lysed and WCLs were immunoprecipitated with an GFP antibody, followed by immunoblotted. C) BMDMs were co-transfected with the indicated combinations of the constructs. Cells were lysed and WCLs were IP with MYC antibody, followed by immunoblotted.

FIG. 24 illustrates that ABCF1 targets TRAF3 for K63-polyubiquitination. A) BMDMs were co-transfected with the indicated combination of the constructs, and processed as in (FIG. 23C). B) WCLs were immunoprecipitated with ABCF1 antibody and immunoblotted from Trif siRNA-treated BMDMs in presence and absence of LPS.

FIG. 25 illustrates that ABCF1 targets TRAF3 for K63-polyubiquitination and controls SIRS to ET phase transition during sepsis. A) Heatmap represents fold change of various serum cytokines and chemokines, p-value<0.01. B) Percentage survival of both NETT and ETT treated WT and Abcf1+/− mice after second LPS injection with n=9 mice per group was monitored until 96 hours, p-value <0.001. C) BMDM WCLs from NETT and ETT mice were analyzed for the expression of regulatory proteins, phospho-proteins and caspases. D) BMDM WCLs were immunoprecipitated with TRAF3 antibody. The immunoprecipitates were immunoblotted with TRAF3 and polyubiquitin antibodies. E) BMDM WCLs were separated by native PAGE gel and were analyzed for IRF3 dimerization. All cytokine and protein analysis were done after 3 hours of second LPS injection.

FIG. 26 illustrates that hypotension induced kidney failure led to increased mortality in Abcf1+/− mice. A) Cytoplasmic and nuclear fractions from BMDMs of ETT and NETT treated WT and Abcf1+/− mice were analyzed by immunoblotted. B) Heatmap representing fold change of MAPK- and ISG-specific phospho-protein and transcription factor expression in WCLs prepared from BMDMs derived from NETT and ETT mice, p-value<0.01. Serum expression of C) Procalcitonin, D) L-Lactate, E) Creatinine from ETT and NETT treated WT and Abcf1+/− mice were measured through ELISA.

FIG. 27 is related to FIG. 20 and illustrates that ABCF1 regulates macrophage polarization to the TRIF-dependent M2 phenotype. Briefly, ABCF1 was silenced with specific siRNA in BMDMs in the presence or absence of various TLR agonists. A) Heatmap representing fold change of various cytokines and chemokines in BMDM cell culture supernatants after TLR2 agonist peptidoglycan (100 ng/ml for 24 hours), TLR9 agonist CpG DNA (5 μM) for 24 hours and TLR3 agonist Poly I:C (10 μg/ml) for 24 hours stimulation, p-value<0.01. Heatmap was generated and fold change calculations were done as indicated in materials and methods. ABCF1 was silenced with specific siRNA in BMDMs in the presence or absence of LPS (100 ng/ml) for 30 mins, B) Cytoplasmic and nuclear fractions were analyzed by immunoblot for the expression and phosphorylation of the indicated transcription factors involved in macrophage polarization. C) WCLs from BMDMs treated with Abcf1-specific siRNA in the presence or absence of LPS, and D) ABCF1-overexpressing BMDMs in the presence or absence of LPS, were immunoblotted with specific antibodies to detect expression of regulatory proteins, kinases, and transcription factors. E) BMDMs were treated with recombinant TNFα (10 ng/ml) for 24 hours, recombinant IL-1β (10 ng/ml) for 24 hours, recombinant IL-10 (50 ng/ml for 24 hours) and recombinant IFNβ (10,000 IU/50 μl) for 24 hours and whole cell lysates were prepared and immunoblotted with ABCF1 antibody.

FIG. 28 is related to FIGS. 20, 22 and 24. ABCF1 is necessary for TLR4 endocytosis TLR4 endocytosis was analyzed by flow cytometry in BMDMs A) Stimulated with various LPS concentrations for 30 mins. B) Stimulated with LPS at 10 ng/ml concentration for various time points. C) Treated BMDMs were stimulated with LPS (100 ng/ml) for 30 mins and D) without LPS. E) Both WT and Abcf1+/− mice were intraperitoneally (i.p.) injected with 50 μg LPS, and after various time points, mice were euthanized and the spleens harvested. TLR4 surface expression on CD11b+ and F4/80+ splenic macrophages were measured using flow cytometry (n=9 mice per group). Bar graphs represent mean fluorescence intensity (MFI), error bars represent standard deviation and p-values are indicated.

FIG. 29 is related to FIGS. 22, 23 and 24 and illustrates that ABCF1 forms a complex with SYK, TRAF6 and TRAF3 during LPS-TLR4 signaling A) BMDMs were treated with cIAP1/2-specific siRNA in the presence or absence of LPS (100 ng/ml) for 30 mins. Cells were lysed and WCLs were immunoblotted with ABCF1, TAK1, p-TAK1, and cIAP1/2 antibodies. B) BMDMs were stimulated with LPS (100 ng/ml) for 30 mins and WCLs were immunoprecipitated with anti-ABCF1 and anti-IgG antibodies. The immunoprecipitates were immunoblotted with ABCF1, SYK, TRAF6, TRIF and TRAF3 antibodies. C) SYK was overexpressed (Over Exp.) in BMDMs, WCLs were immunoprecipitated with SYK antibody, and the immunoprecipitates were immunoblotted with anti-ABCF1 and SYK antibodies. D) TRAF3 was overexpressed in BMDMs, WCLs were immunoprecipitated with TRAF3 antibody, and the immunoprecipitates were immunoblotted with ABCF1 and TRAF3 antibodies. E) Protein analysis of p-SYK and p-PLCγ2 in Abcf1 siRNA-treated BMDMs in presence or absence of LPS.

FIG. 30 is related to FIGS. 25 and 26 and illustrates that ABCF1 is necessary for TRIF dependent transition from SIRS to ET phase and apoptosis during sepsis. A) Splenic macrophages from ETT and NETT treated WT and Abcf1+/− mice were analyzed for A20 and TAK1 phosphorylation. B) BMDM WCLs from untreated WT, Abcf1+/− and the sepsis-induced mice at various time points after their mortality were immunoblotted with ABCF1 and IRF3 antibodies. C) Heatmap representing fold change of phosphorylation of cell survival proteins and various Src family kinases in WCLs prepared from BMDMs derived from NETT and ETT mice, p-value<0.01. Heatmap was generated and fold change calculations were done as indicated in materials and methods. D) WCLs from Abcf1 siRNA-treated BMDMs stimulated with LPS (100 ng/ml for 30 mins) were analyzed for the expression of proteins associated with apoptosis (CASP-3) and pyroptosis (CASP-1, NLRP3, ASC) by immunoblotting. E) ETT and NETT WT and Abcf1+/− mice were perfused and sacrificed after 3 hours of second LPS injection. Kidney tissues were fixed in 4% paraformaldehyde for 24 hours, washed in PBS and embedded in paraffin. 5-μm-thick sections were cut and were stained with hematoxylin and eosin (HE). The slides were seen under Olympus BX51 microscope and images were taken with Olympus cellSens software. Scale bar 20 μm. Black arrows represent areas of red blood cells sludging.

FIG. 31 is related to FIGS. 25 and 26 and illustrates that sepsis-induced pathogenesis in the bone marrow cells is ABCF1 dependent. A) Bone marrow transplant efficiency was greater than 95% in all the recipient mice. Representative flow cytometric plots of peripheral blood cells depict bone-marrow transplantation (BMT) CD45.1 vs CD45.2 status of donor and recipient mice both pre-BMT and 8-weeks post-BMT. B) Heatmap represents fold change of various serum cytokines and chemokines from untreated WT, Het and bone marrow transplanted mice, p-value<0.01. Heatmap was generated and fold change calculations were done as indicated in materials and methods. C) Percentage survival of both NETT and ETT treated bone marrow transplanted mice after second LPS injection with n=10 mice per group was monitored until 96 hours, p-value <0.001. Expression of D) Procalcitonin, E) L-Lactate, F) Creatinine in serum from ETT and NETT treated bone marrow transplanted mice were measured through ELISA.

### DETAILED DESCRIPTION

The present invention is based on the discovery that ABCF1 is an E2 ubiquitin-conjugating enzyme that acts as an innate immune regulator by targeting key inflammatory pathway proteins for polyubiquitination. It was also discovered that ABCF1 plays a role in controlling production of pro-inflammatory cytokines and the shift from systemic inflammatory response (SIRS) phase to endotoxin tolerance (ET) phase of sepsis. Given this activity and involvement, the present invention provides methods and compositions for modulating inflammation and/or an immune response by modulating activity and/or expression of ABCF1. A worker skilled in the art would readily appreciate that depending on the particular disease or condition that it may be desirable to either stimulate or inhibit inflammation and/or an immune response. For example, inhibition of inflammation and/or an immune response may be useful in the prevention and/or treatment of inflammatory or autoimmune diseases or disorders while stimulation of an immune response may be useful in the prevention and/or treatment of infection and/or cancer. Accordingly, the present invention provides ABCF1 antagonists and agonists for use in the treatment of diseases and disorders.

In certain embodiments, there is provided a method of stimulating an inflammatory response and/or an immune response by inhibiting the expression and/or activity of ABCF1. Non-limiting examples of methods to inhibit expression and/or activity a polypeptide interest such as ABCF1 include administration of an antagonist, including but not limited to small molecules, antibodies against the polypeptide of interest and nucleic acids such as antisense oligonucleotides or siRNA which target the nucleic acids which encode ABCF1.

In certain embodiments, the stimulation of an immune response by inhibiting ABCF1 may be used in the treatment of cancer and/or stimulation of an immune response against a pathogen. Pathogens include but are not limited to viruses and bacteria.

In certain embodiments, there is provided a method of inhibiting an inflammatory response and/or an immune response by upregulating the expression and/or activity of ABCF1. Non-limiting examples of methods to enhance expression and/or activity a polypeptide interest, such as ABCF1, include administration of the polypeptide of interest, administration of a nucleic acid or vector which encodes the polypeptide of interest or administration of one or more molecules which enhance expression of the polypeptide of interest. Appropriate vectors are known in the art and include but are not limited to adenoviral vectors. Escitalopram is a known enhancer of the ABCF1 pathway.

In certain embodiments, there is provided a method of preventing and/or treating sepsis by stimulating the expression and/or activity of ABCF1. In certain embodiments, there is provided a method of treating an inflammatory or autoimmune disease by stimulating the expression and/or activity of ABCF1. The diseases include but are not limited to inflammatory bowel disease including but not limited to Crohn's disease, ulcerative colitis, arthritis including but not limited to autoimmune arthritis such as Rheumatoid Arthritis, multiple sclerosis, pancreatitis and diabetes.

Previously, it has been determined that ABCF1 extrinsically regulates phagocytosis. Accordingly, in certain embodiments, a soluble form of ABCF1 is used to regulate phagocytosis. In specific embodiments, soluble ABCF1 is a phagocytic ligand. In specific embodiments, soluble ABCF1 promotes phagocytosis of apoptotic cells.

In certain embodiments, there is provided a soluble ABCF1. The soluble ABCF1 may be utilized in the treatment of diseases. In certain embodiments, soluble ABCF1 is utilized to modulate an inflammatory response and/or an immune response. In certain embodiments the soluble ABCF1 acts as an agonist. In such embodiments where ABCF1 acts as agonist, the soluble ABCF1 may be used in the treatment of diseases which require an inhibition of an inflammatory response and/or an immune response. Such diseases include for example sepsis and autoimmune diseases. In certain embodiments, the soluble ABCF1 acts as an antagonist. In such embodiments where ABCF1 acts as antagonist, the soluble ABCF1 may be used in the treatment of diseases which require stimulation of an inflammatory response and/or an immune response.

ABCF1 may be used as a biomarker. The ABCF1 protein and nucleic acid sequences (genomic and cDNA) are known in the art. See for example GenBank Accession numbers AQY76226.1, AQY76225.1, KY500135.1 and KY500134.1. Methods of measuring gene expression including mRNA and protein expression are known in the art. In certain embodiments, decreased expression of ABCF1 is indicative of an inflammatory and/or immune response. In certain embodiments, increased expression of ABCF1 is indicative of a decreased inflammatory and/or immune response. Accordingly, ABCF1 expression may be used as a biomarker for diseases or disorders associated with increased or decreased inflammatory and/or immune responses. ABCF1 expression may be used in methods of determining clinical outcome of diseases and/or disorders associated with increased or decreased inflammatory and/or immune responses. Accordingly, in certain embodiments, the present invention provides a method of determining clinical outcome of diseases and/or disorders associated with increased or decreased inflammatory and/or immune responses by determining expression of one or more genes including Abcf1.

ABCF1 may be used as a biomarker for inflammation and/or immune response associated with infection, autoimmune diseases, inflammatory diseases and/or cancer. ABCF1 may also be used as a biomarker for cancer. In particular, it is known in the art that elevated microRNA-23a expression enhances the chemoresistance of colorectal cancer cells with microsatellite instability to 5-Fluorouracil by directly targeting ABCF1 (Li et al. Current Protein and Peptide Science 16(4):301-309). It is also known that ABC transporter genes are down regulated in prostate cancer) Demidenko et al BMC Cancer. 2015; 15: 683).

In certain embodiments, ABCF1 is used as a biomarker for inflammation and optionally cell survival during sepsis. In specific embodiments, the ABCF1 is soluble ABCF1. Accordingly, in certain embodiments, the present invention provides a method of determining clinical outcome of a sepsis patient by determining expression of one or more genes including Abcf1.

In certain embodiments, ABCF1 is used as biomarker to determine clinical outcome of inflammatory bowel disease including but not limited to Crohn's disease and ulcerative colitis.

In certain embodiments, there is provided bioassay screens which utilize ABCF1 to identify new drugs. For example, the screens may be used to identify drugs that modulate an immune response (including an anti-pathogen or anti-cancer immune response, reduce inflammation, treat autoimmune disease.

In certain embodiments, there is provided methods to determine ABCF1 expression. Such methods may be used to identify agents that modulate ABCF1 expression and therefore may be useful in the identification of drugs. In specific embodiments, a reporter gene is placed under the control of the ABCF1 promoter and the reporter gene product is measured (either qualitatively or quantitatively). Cells, including but not limited macrophages such as RAW 264.7 cell line, comprising the ABCF1 promoter reporter gene product may be used in assays to identify agents that modulate ABCF1 expression.

To gain a better understanding of the invention described herein, the following examples are set forth. It will be understood that these examples are intended to describe illustrative embodiments of the invention and are not intended to limit the scope of the invention in any way.

### Example 1: A Novel Ubiquitin E2 Conjugating Enzyme ABCF1 Inversely Regulates TLR4 Endocytosis and Cytokine Storm During Sepsis

ABCF1 inversely regulates inflammation by TLR2 and 9. It associates with OAS1 and regulates dsRNA viral cleavage and controls viral infection. After LPS stimulation, ABCF1 modulates ITAM phosphorylation in FcγR II A and regulates E. coli phagocytosis in BMDM.

### Material and Methods

Mice and cells: Bone marrow cells were isolated from femur and tibia of C57BL/6 mice and were matured into macrophages as described (Trouplin et al. 2016). Abcf1+/− mice (Het) mice were generated as previously described (Wilcox et al. 2017) and C57BL/6J (Jackson Laboratories; 000664) mice were used as wild type (WT) controls for in vivo experiments.

Transfections: All gene knockdowns in cells were done via RNAi interference using Lipofectamine™ RNAiMAX Transfection Reagent (ThermoFisher Scientific; 13778075) according to manufacturer's instructions and the extent of knockdown was checked by western blot. Mouse Abcf1 siRNA (sc-140760), mouse cIAP2 siRNA (sc-29851), mouse Trif siRNA (sc-106845) were from Santa Cruz Biotechnology. All gene overexpressions were done using Lipofectamine™ 2000 Transfection Reagent (ThermoFisher Scientific; 11668027) according to manufacturer's instructions and the extent of overexpression was checked by western blot. Mouse gene plasmids: Mouse Abcf1WT expression vector was purchased from GenScript (Clone Id: OMu73202). The Abcf1 C647S mutation and GFP tag was cloned both in Abcf1 WT and Abcf1 C647S by Top Gene Technologies (Montreal, Canada). FLAG-Traf6 was a gift from John Kyriakis (Addgene plasmid #21624). pMSCV-mCherry-Syk was a gift from Hidde Ploegh (Addgene plasmid #50045). Mouse Traf3 expression vector was purchased from Origene (MR225761). Ubiquitin plasmids: pRK5-HA-Ubiquitin-WT (Addgene plasmid #17608) contains the wild type ubiquitin with all lysines intact, pRK5-HA-Ubiquitin-K48 (Addgene plasmid #17605) and pRK5-HA-Ubiquitin-K63 (Addgene plasmid #17606) showed 100% similarity with mouse ubiquitin C gene. Ub-K48 and Ub-K63 plasmids contain lysine at residues 48 and 63 only; all other lysine were mutated to arginine.

Cytokine Analysis: Cell culture supernatant or blood serum were prepared and incubated on nitrocellulose membranes containing different anti-cytokine antibodies printed in duplicates provided with Proteome Profiler Mouse Cytokine Array Kit, Panel A (R&D Systems; ARY006). Cytokine levels are depicted as heat maps as described below.

Co-immunoprecipitation: 2×10⁷ cells were lysed with freshly prepared 20 mM Tris HCl pH 8.0, 137 mM NaCl, 1% Nonidet P-40, 2 mM EDTA, 1× Halt protease and phosphatase inhibitor cocktail (ThermoFisher Scientific; 78440) and 20 mM N-Ethylmaleimide (Santa Cruz Biotechnology; sc-202719). Protein A or protein G beads were washed twice with wash buffer comprising 10 mM Tris HCl pH 7.4, 150 mM NaCl, 1% Nonidet P-40, 1 mM EDTA, 1× Halt protease and phosphatase inhibitor cocktail and 20 mM N-Ethylmaleimide, centrifuged at 3,000×g for 2 minutes at 4° C. and incubated with indicated antibody for 4 hours at 4° C. on a rotating shaker. Beads were then washed twice and incubated with the cell lysate overnight at 4° C. Beads were washed and the complex was eluted by acidification using 3×50 μl 0.1 M glycine at pH 2 by incubating the sample for 10 minutes with frequent agitation before gentle centrifugation. The eluate was neutralized by adding equal volumes of Tris HCl pH 8.0.

Western blotting: Cell lysates were prepared as described above. All co-immunoprecipitation samples were prepared without DTT in the sample buffer, unless otherwise mentioned. All other western blot samples were prepared with DTT in the sample buffer. Lysates were electrophoresed and immunoblotted with indicated antibodies.

### Antibodies and Chemicals:

Antibodies used for western blots and immunoprecipitation, anti ABCF1 antibody for western blotting (ThermoFisher Scientific; PA5-29955), anti ABCF1 antibody for co-immunoprecipitation (Proteintech; 13950-1-AP), anti I-KB antibody (abcam; ab32518), phospho-l-KB antibody (abcam; ab12135), anti NF-KB p65 (abcam; ab16502), anti phospho-NF-KB p65 (abcam; ab86299), anti-cleaved CASP1 antibody (Santa Cruz Biotech; sc-514), anti CASP1 antibody (abcam; ab108362), anti-cleaved CASP3 (Cell Signaling Technology; 9661), anti CASP3 antibody (abcam; ab13847), anti NLRP3 antibody (R&D Systems; MAB7578), anti ASC antibody (Santa Cruz Biotech; sc-514414), anti TAK1 antibody (abcam; ab109526), anti phospho-TAK1 antibody (abcam; ab192443), anti TBK1 antibody (abcam; ab40676), anti phospho-TBK1 antibody (abcam; ab109272), anti IRF3 antibody (Santa Cruz Biotech; sc-15991), anti phospho-IRF3 (Cell Signaling Technology; 4947), anti SYK antibody (Santa Cruz Biotech; sc-1240), anti phospho-SYK antibody (abcam; ab58575), anti PLCγ2 antibody (Santa Cruz Biotech; sc-5283), anti phospho-PLCγ2 antibody (Cell Signaling Technology; 3871), anti A20 antibody (Santa Cruz Biotech; sc-69980), anti K48 antibody (Cell Signaling Technology; 4289), anti K63 antibody (Cell Signaling Technology; 5621), anti TRAF6 antibody (Santa Cruz Biotech; sc-8409), anti cIAP antibody (Santa Cruz Biotech; sc-1869), anti TRIF antibody (abcam; ab13810), anti TRAF3 antibody (Santa Cruz Biotech; sc-6933), anti UB antibody (Santa Cruz Biotech; sc-8017), anti GAPDH antibody (abcam; ab181602), anti-Histone H3 antibody (abcam; ab8580), anti GFP antibody (abcam; ab290), anti MYC antibody (abcam; ab9132), anti mCherry antibody (ThermoFisher Scientific; PAS-34974), anti HA antibody (Santa Cruz Biotech; sc-7392). Antibodies for fluorescence associated cell sorting (FACS), CD86 (BioLegend; 105011), CD206 ((BioLegend; 1417097), MHC-II (BioLegend; 141607), TLR4 (BioLegend; 145406), CD11b (BioLegend; 101223). Chemicals used, NP-40 (abcam; ab142227), Smac Mimetic (Tocris; 5141), Dynamin inhibitor I Dynasore (Santa Cruz Biotech; sc-202592), Lipopolysaccharide (Santa Cruz Biotech; sc-221855), N-Ethylmaleimide (Santa Cruz Biotech; sc-202719), PMSF solution (Santa Cruz Biotech; sc-482875) and Aprotinin (Santa Cruz Biotech; sc-3595).

Phospho-kinase Analysis: Cell lysates were prepared as described above. Lysates were incubated on nitrocellulose membranes containing different antibodies printed in duplicates provided with Proteome Profiler Human Phospho-Kinase Array Kit (R&D Systems; ARY003B). This kit can also be used for mouse samples and has previously been tested by (O'Hara et al. 2016; Chen et al. 2010). Phospho-kinase levels are depicted as heat maps as described below.

IRF3 dimerization assay: Cells were lysed in a buffer containing 50 mM Tris, pH 8.0, 1% NP40, 150 mM NaCl, 1 mM PMSF and 20 μg/ml of aprotinin, and supplemented with native PAGE sample buffer (125 MTris, pH 6.8, and 30% glycerol). The samples were separated by native PAGE and analyzed by immunoblotting.

Cytoplasmic and Nuclear fractionation: Bone marrow macrophages were stimulated with mentioned treatments and cytoplasmic and nuclear fractionation was performed with NE-PER Nuclear and Cytoplasmic Extraction Reagents (Thermo Scientific; 78833) according to manufacturer's instructions.

In vitro ubiquitination Assay: Abcf1WT and Abcf1C647S expression plasmids were overexpressed in macrophages as described above. Before overexpressing Abcf1C647S expression plasmid, cells were treated with Abcf1 siRNA, as described above to eliminate endogenous Abcf1 levels. Abcf1 siRNA treated cells were washed with cold PBS twice and then Abcf1C647S expression plasmid was overexpressed. Cell lysates were prepared and subjected to co-immunoprecipitation with anti ABCF1 antibody. Extend of overexpression and co-immunoprecipitation was checked by western blot. Ubiquitination Assay was performed on immunoprecipitated samples with Ubiquitylation Assay Kit (abcam; ab139467) according to manufacturer's instructions. Samples were run on a gel and blotted with anti ABCF1 and anti UB antibodies.

In vivo Endotoxin Tolerance: All in vivo experiments were approved and were performed in accordance with University of British Columbia Animal care Committee. 8-10 weeks old female WT and Abcf1+/− mice were either pre-treated with i.p injections of 0.5 mg/kg LPS or with sterile normal saline. 24 hours later, all mice received an i.p. injection of 20 mg/kg LPS. The former group with two LPS injection was named endotoxin treated (ETT) and the latter group with one PBS and one LPS injection was named non-endotoxin treated (NETT). Mice were either euthanized (by 3% isoflurane in O₂ followed by CO₂ inhalation) 3 hours after the second injection, to obtain blood and organs or were monitored every 8 hours for 4 days for survival. Serum was isolated from blood and cytokines were measured.

Survival Study: Both WT and Abcf1+/− mice were monitored once a day after the first LPS or PBS injection. After the second LPS injection, mice were monitored every 8 hours and were scored on a (0-3) scale based on severity of behaviour and activity, appearance, grooming and posture, respiratory rate, effort and pattern, rectal temperature, pain and body weight. Score of 3 are considered humane endpoint (were euthanized) and include: no palpable fat over sacroiliac region, severely reduced muscle mass, prominent vertebrae & iliac crests. Immobile, not moving when nudged or picked up; animal cannot right itself if placed on side. Piloerection (>75% of body), matted appearance, unkempt and ungroomed with other severe signs of illness. Constant, severe hunching when sitting and walking and non-responsive to treatment. Gasping or open mouth breathing either in cage or when being weighed; struggling to breathe and unable to move around due to difficulty breathing; noisy breathing (squeaking with effort to breathe), <33° C. for three consecutive measurements one hour apart, persistent/constant signs of pain that interfere with normal function despite analgesia administration, animal unresponsive and cold to touch, severe skin tent (>10 seconds).

Histological Analysis: Mice were first anesthetised by ketamine/xylazine mixture (up to 150 mg/kg body weight ketamine and 15 mg/kg body weight xylazine) administered via i.p. injection and then perfused with sterile PBS at a flow rate of 5 ml/minute for 5 minutes and were sacrificed 3 hours after the second LPS injection. Kidney, brain and heart tissues were fixed in 4% paraformaldehyde for 24 hours, washed in PBS and imbedded in paraffin. 5 μm thick sections were cut and were stained with hematoxylin and eosin (HE). Snapshots of histology were taken using an Olympus BX51 microscope equipped with a 40× objective. Images were generated using an attached Olympus DP73 camera and the high-performance Olympus cellSens software.

Statistical Analysis: The data are presented as the mean±SD. The means were compared by paired or unpaired student t-tests. P values <0.05 were considered significant. Heat maps were generated in GraphPad prism software (GraphPad PRISM Software, San Diego, Calif.). Survival is presented as Kaplan-Meier curves and differences were assessed by the log-rank test in GraphPad prism software. Sample size for each experiment is provided in the figure legend.

### Results

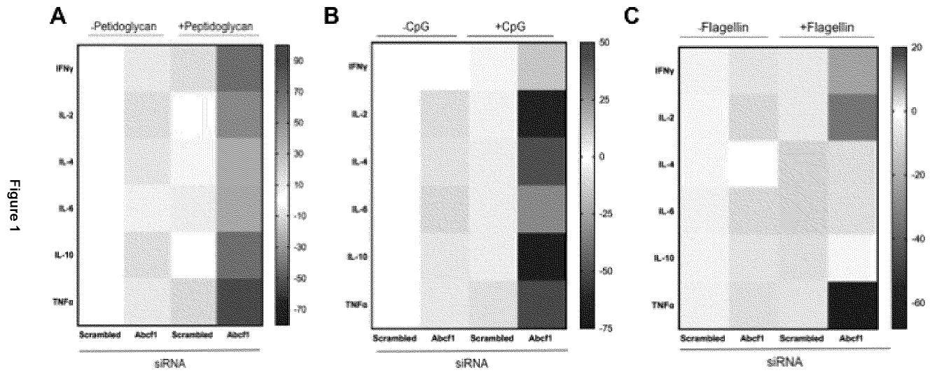

ABCF1 negatively regulates pro-inflammatory cytokines in TLR2 and TLR9 signaling and positively regulates anti-inflammatory cytokines in TLR5 signaling: Binding of a PAMP to a TLR, leads to production of myriad of cytokines and chemokines. After the insult, the downstream effector response helps modulate the immune reaction and regulates several signaling. To investigate if ABCF1 regulates TLR signaling by modulating cytokine levels, ABCF1 was knocked down through RNAi in BMDM and levels of both pro- and anti-inflammatory cytokines were analyzed. On stimulating Abcf1 siRNA treated BMDM with TLR2 agonist peptidoglycan, levels of pro-inflammatory cytokines like IL-6 and TNFα were significantly elevated by 15-fold and 80-fold respectively (FIG. 1 A). IFNβ levels were also found to be upregulated by 70-fold. Whereas, levels of anti-inflammatory cytokines like IL-2, IL-4, IL-10 were significantly down-regulated by 30-fold, 20-fold and 50-fold respectively (FIG. 1 A). Same trend in cytokine levels was observed when Abcf1 siRNA treated BMDM were stimulated with TLR9 agonist CpG DNA. Upon stimulation, IL-6 and TNFα were significantly elevated by 30 and 50-fold respectively and IL-2, IL-4 and IL-10 were down-regulated 50-60-fold (FIG. 1 B). On the other hand, on stimulating Abcf1 siRNA treated BMDM with TLR5 agonist flagellin, levels of IL-6 and TNFα were down regulated by 10-fold and 60-fold respectively and anti-inflammatory cytokines like IL-4 was slightly upregulated by 5-fold and IL-10 was upregulated by 2 fold (FIG. 1 C). This suggests that ABCF1 negatively regulates inflammation through TLR2 and TLR9 signaling and positively regulates inflammation through TLR5 signaling.

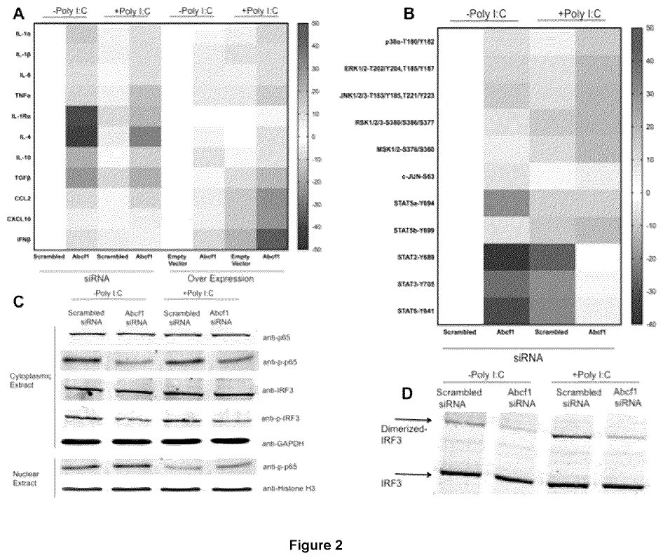

ABCF1 is necessary for IFN I specific chemokine and cytokine production after TLR3 agonist Poly I:C stimulation: Type I interferons (IFN-I) are critical for host defense against viruses. Once a cell is infected by a viral infection, a myriad of IFN-I specific genes is produced that help in elimination of viral infection. To identify if ABCF1 is necessary for this mechanism, ABCF1 was knocked down in BMDM through RNAi and the levels of various IFN-I proteins and transcription factors were studied. Levels of IFN-I proteins such as CCL2, CXCL10, IFNβ were down regulated 5 to 10-fold when treated with both Abcf1 siRNA and dsRNA agonist, poly I:C (FIG. 2 A). This trend was completely reversed when ABCF1 was overexpressed in BMDM. These proteins were 20 to 40-fold upregulated after ABCF1 overexpression and poly I:C stimulation (FIG. 2A).

On the other hand, pro-inflammatory cytokines like IL-1β, IL-6 and TNFα were marginally elevated after Abcf1 siRNA treatment, but their levels were significantly up regulated by 15 to 25-fold after Abcf1 overexpression and poly I:C stimulation (FIG. 2 A). Anti-inflammatory cytokines like IL-4 were significantly down regulated by 27-fold when treated with Abcf1 siRNA and poly I:C treatment, but after overexpression, the down regulation was only found to be 6-fold. IL-10 on the other hand was slightly down regulated when treated with both Abcf1 siRNA and poly I:C, but was significantly elevated when ABCF1 was overexpressed (FIG. 2 A). ABCF1 also positively regulated TGFβ levels after poly I:C stimulation (FIG. 2 A).

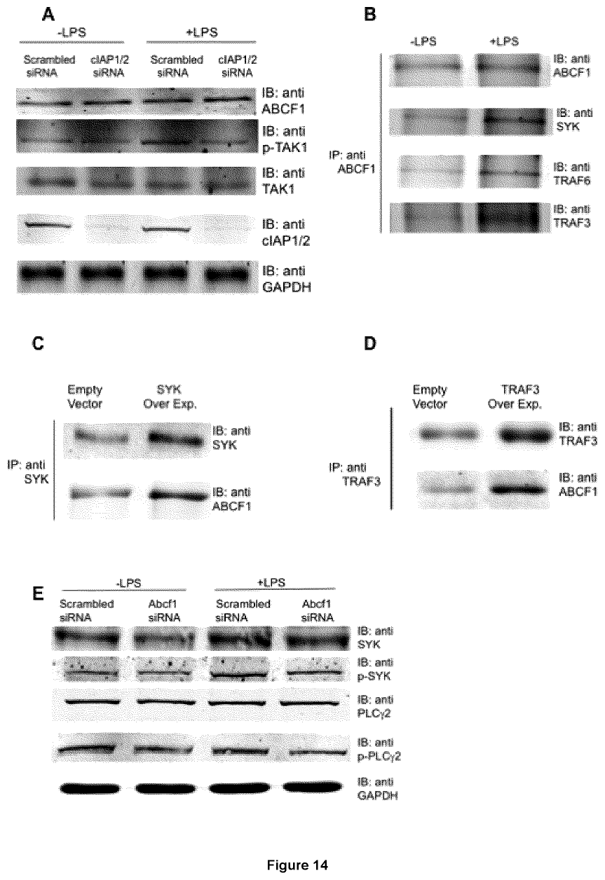

Thus, this cytokine and chemokine regulation by ABCF1 shows that ABCF1 is necessary for eliminating viral infection via IFN-I production. it ABCF1 positively regulates both TGFβ and IL-10 levels after poly I:C stimulation and these cytokines positively regulate viral clearance in presence of ABCF1 (FIG. 14).

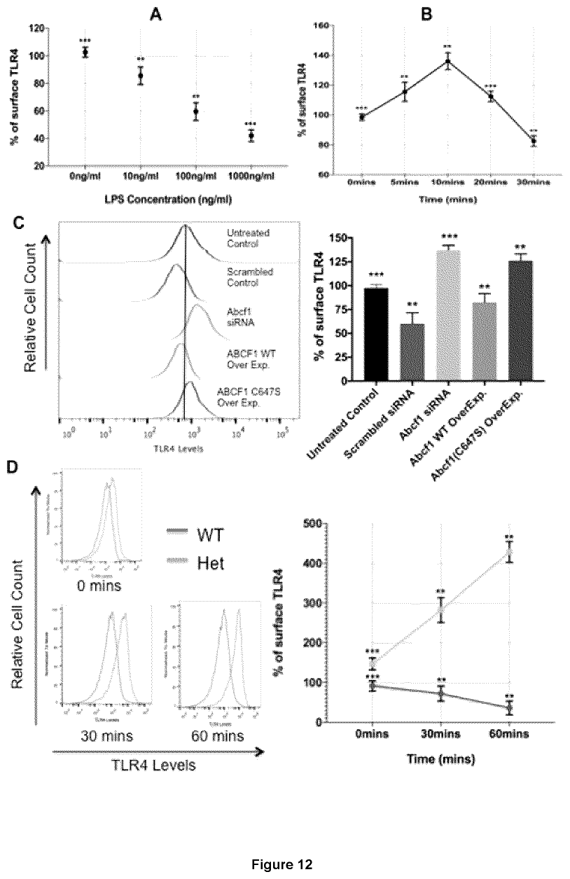

ABCF1 counteracts viral infection via JAK-STAT pathway and IRF3 production: The cell can either inhibit translation by activating Phospho kinase R (PRK) or degrade the mRNA by RNaseL by activating OAS proteins (Gantier and Williams 2007). Both of these pathways lead to activation and dimerization of IRF3, which lead to production of ISGs ((Chakrabarti et al 2011, Sen et al 2011). To study if ABCF1 regulates this signaling, phosphorylated levels of ISG specific STAT proteins like STAT2, STAT3 and STAT6 in Abcf1 siRNA treated and dsRNA agonist, poly I:C stimulated BMDM were analyzed. Significantly reduced phosphorylation levels of these proteins were observed when treated with Abcf1 siRNA alone, the levels elevated by 30 to 40-fold after poly I:C stimulation alone, but showed only 5 to 10-fold increase when treated with both Abcf1 siRNA and Poly I:C (FIG. 12B). Slight increase in MAPK pathway was also seen with 5 to 7-fold elevation in phosphorylation levels of p38, ERK, JNK and transcription factor c-JUN (FIG. 12B). Reduced IRF3 dimerization was observed in Abcf1 siRNA treated BMDM. IRF3 phosphorylation and dimerization significantly increased when stimulated with poly I:C, but levels plummeted again when treated with both Abcf1 siRNA and poly I:C (FIG. 12C, D). This suggests that ABCF1 is necessary for dsRNA mediated signaling via STAT proteins and leads to IRF3 dimerization and production of ISGs (FIG. 14).

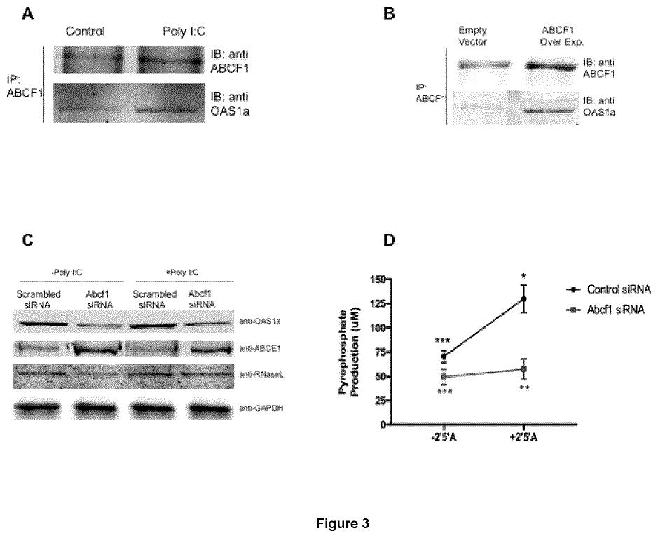

ABCF1 is necessary for OAS1a and RNaseL activity after Poly I:C stimulation: The anti-viral OAS1-RNaseL pathway has shown to be crucial for viral RNA cleavage and production of IFN-I (Meng et al 2012). To investigate, if ABCF1 regulates OAS1a signaling, poly I:C stimulated BMDM were immunoprecipitated with anti ABCF1 antibody and data revealed association between ABCF1 and OAS1a (FIG. 3 A, B). BMDM were then treated with Abcf1 siRNA in presence and absence of poly I:C and OAS1a protein levels were analyzed. Western blot analysis showed decreased OAS1a levels when treated with Abcf1 siRNA alone and no significant increase was seen when stimulated with poly I:C (FIG. 3C).

To examine if ABCF1 was also necessary for OAS1 anti-viral activity, the amount of pyrophosphate (PPi) produced as a by-product of 2′5′A formation was quantified. Abcf1 siRNA treated and poly I:C stimulated BMDM showed no increase in PPi production when stimulated with OAS1. On the other hand, 2-fold elevation in PPi levels was seen in controls (FIG. 3D).

Since downstream OAS signaling leads to RNaseL activation and viral degradation, it was investigated if ABCF1 regulated RNaseL levels. RNaseL protein levels were measured in Abcf1 siRNA treated and poly I:C stimulated BMDM. Immuno blot analysis showed that ABCF1 positively regulates RNaseL levels in poly I:C stimulated BMDM. (FIG. 3C).

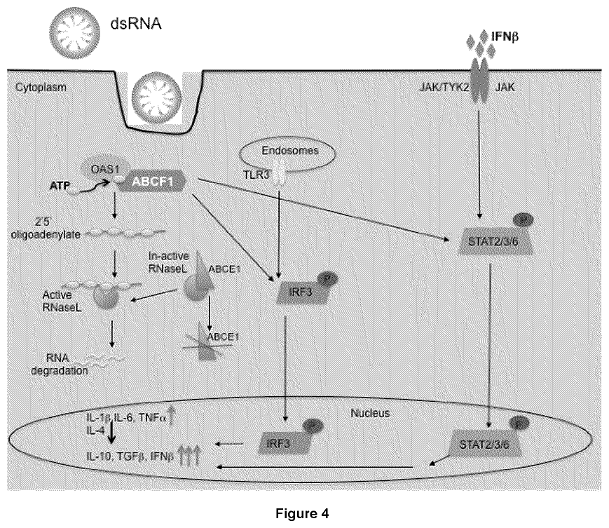

Western blot analysis showed that ABCF1 negatively regulates ABCF1 levels in BMDM treated with Abcf1 siRNA in presence and absence of Poly I:C (FIG. 3C). This confirms that ABCF1 positively regulates viral degradation by RNaseL by negatively regulating ABCF1 (FIG. 4).

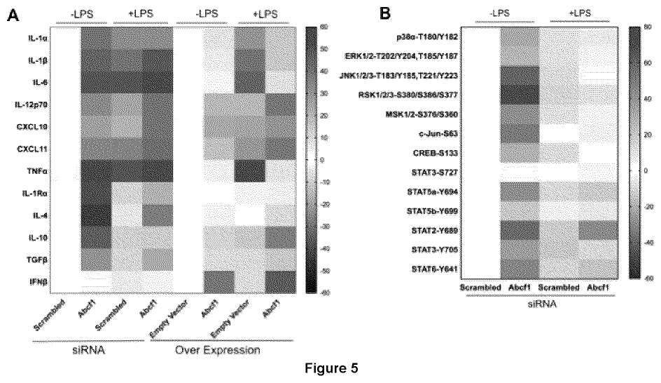

ABCF1 is necessary for anti-inflammatory cytokine production after LPS stimulation: To investigate if ABCF1 regulates LPS signaling, BMDM were treated with Abcf1 siRNA and ABCF1 over expression vector in presence and absence of LPS and levels of pro- and anti-inflammatory cytokines were measured. 20-30 fold increase in pro-inflammatory cytokines like IL-6, TNFα, IL-1β, IL-12p70, CXCL10, CXCL11 (FIG. 5A) was observed when cells were treated with Abcf1 siRNA only and a further increase was seen when cells were stimulated with LPS, with IL-6 and TNFα showing more than 60 fold elevation. On the other hand, anti-inflammatory cytokines like IL-1Rα, IL-4, IL-10 and ISG's like IFNβ showed 30-fold downregulation with Abcf1 siRNA treatment alone and the increase was not profound even after LPS stimulation (FIG. 5A).

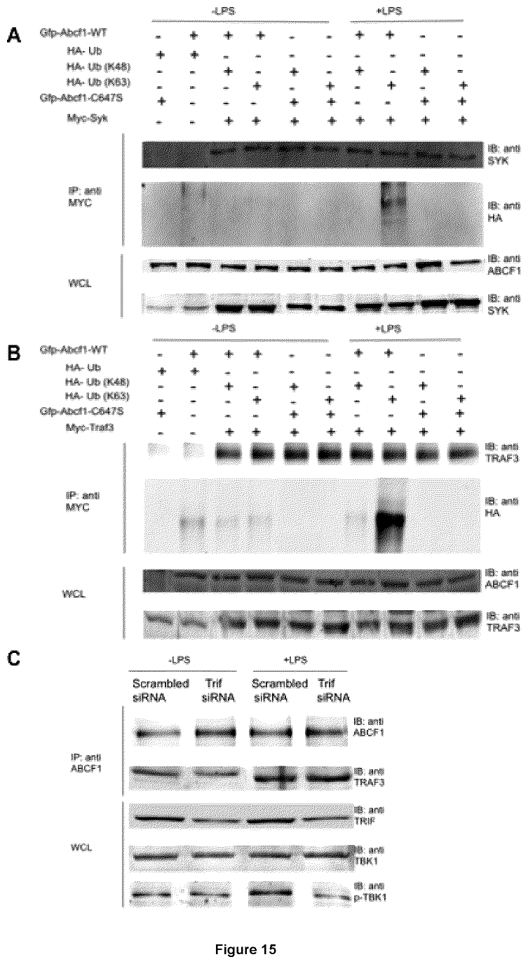

On the other hand, when ABCF1 was overexpressed a slight increase was observed in IL-6, TNFα, IL-1β but IL-12p70, CXCL10 and CXCL11 were found to be significantly downregulated. This trend was even more profound when cells were stimulated with LPS (FIG. 15A). Elevated levels of anti-inflammatory cytokines like IL-1Rα, IL-4, IL-10 and ISG's like IFNβ were observed with ABCF1 overexpression in BMDM alone and this increase was even more profound when BMDM were stimulated with LPS (FIG. 15A).

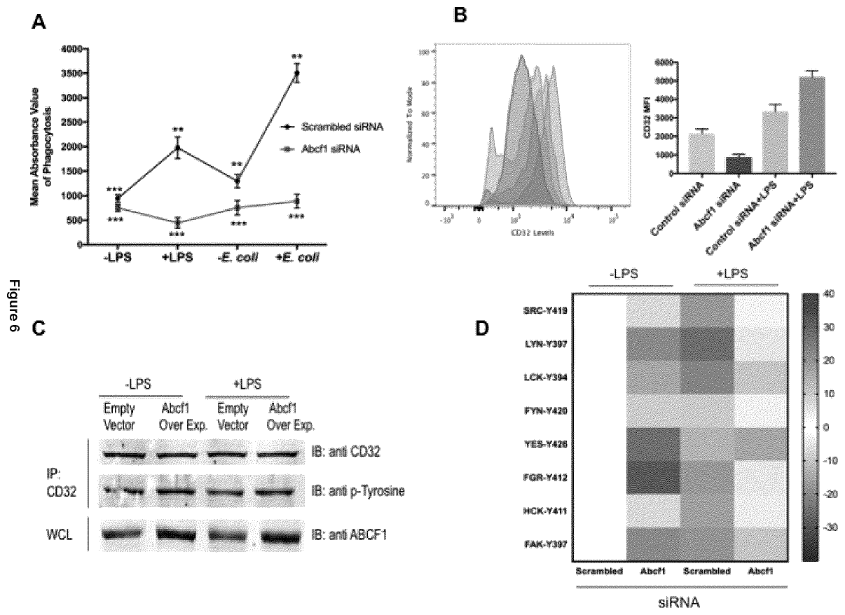

ABCF1 is necessary for FcγR IIA mediated phagocytosis in BMDM: The role of ABCF1 in regulating phagocytosis was investigated by incubating Abcf1 siRNA treated BMDM in presence and absence of LPS with fluorescent labeled latex IgG coated beads. After flow cytometry analysis, 6-fold reduced fluorescent intensity was observed in Abcf1 siRNA treated BMDM when stimulated with LPS. This confirmed that Abcf1 is essential for phagocytosis of latex IgG coated beads after LPS stimulation (FIG. 6A).

Phagocytosis of fluorescent-labeled E. coli particles was also checked in Abcf1 siRNA treated BMDM. Absorbance value before and after BMDM incubation with E. coli was measured and levels of phagocytosis was determined. After E. coli incubation, Abcf1 siRNA treated BMDM showed 4-fold-reduced levels of phagocytosis, thus confirming that ABCF1 is also necessary for phagocytosis of E. coli (FIG. 6A).

To investigate if ABCF1 mediated phagocytosis via which FcγR, levels of FcγRI (CD64), FcγRII (CD32) and FcγRIII (CD16) were analyzed via flow cytometry. Surface levels of CD64 and CD16 were not altered with loss of ABCF1 irrespective of LPS stimulation in BMDM (data not shown). The levels of CD32 on the other hand seemed to have been positively regulated by ABCF1 after stimulation with LPS stimulation (FIG. 6B). To analyze how ABCF1 mediates phagocytosis via CD32, the later was immunoprecipiated from ABCF1 overexpressed and LPS stimulated BMDM. Strong protein bands were observed when the immunoprecipitates were immunoblotted with anti CD32 antibody (FIG. 6C). The immunoprecipitates also showed strong protein bands when immunoblotted with anti phosphotyrosine antibody, suggesting CD32 ITAM phosphorylation in presence of ABCF1, thus activating phagocytosis (FIG. 6C, 17).

ABCF1 positively regulates SFK and STAT3 activation and negatively regulates MAPKs during phagocytosis: SFK's like Src, Fyn, Lyn and Fgr, have been reported to be critically involved in phagocytosis. To confirm if ABCF1 controls this mechanism, BMDM were treated with Abcf1 siRNA in presence and absence of LPS and phosphorylation levels of various SFK's were analyzed. Phosphorylation of all SFK's was significantly downregulated with varying levels (FIG. 6D), when cells were treated with Abcf1 siRNA only. The levels increased negligibly in almost all SFK's with the exception of SRC and FYN, which were elevated 3 to 4-fold after LPS stimulation (FIG. 6D).

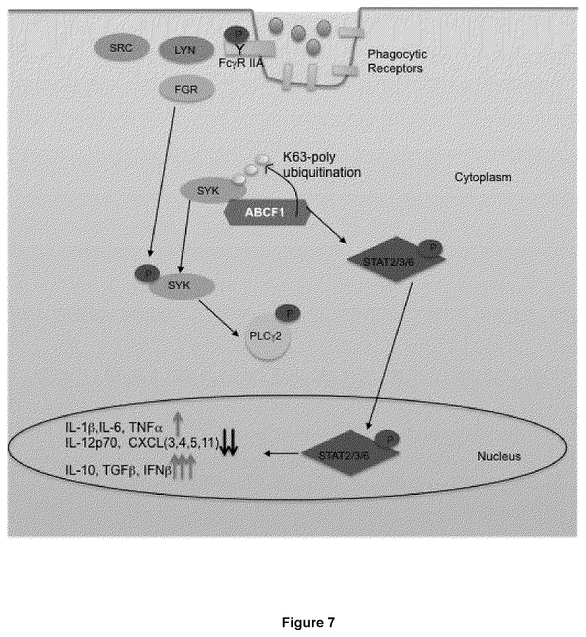

Analysis of protein phosphorylation in BMDM also showed that levels of MAPK's like p38, ERK and JNK were 40 to 50-fold elevated when treated with Abcf1 siRNA and these levels significantly decreased when cells were stimulated with LPS (FIG. 6D). MAPK specific transcription factor c-Jun also showed the same trend (FIG. 6D). STAT transcription factors on the other hand showed an anomalous response. Phosphorylation levels of STAT 5a, STAT 2, and STAT 3 were downregulated with Abcf1 siRNA and the levels seemed to have increased marginally after LPS stimulation (FIG. 6D), whereas STAT 5b phosphorylation was negatively regulated with ABCF1 levels. Thus, it was confirmed that ABCF1 positively regulates SFK and STAT transcription factors in LPS treated BMDM, which aid in phagocytosis (FIG. 7).

### Discussion

The mammalian immune system has evolved to recognize and counteract many different forms of PAMP's through PRR's present either on surface or inside the cell. This convoluted mechanism is under the surveillance of many proteins, which form a vast network of signaling cascades that ultimately control the cells reactions towards invaders. ABCF1 controls immune signaling via TLR2, 3, 4, 5 and 9, which encompasses both gram-positive, gram-negative bacterial, and viral infections. ABCF1 was found to negatively regulate pro-inflammatory cytokines like IL-6 and TNFα production in TLR2, 3, 4 and 9 signaling and positively regulates pro inflammatory cytokine in TLR5 pathway. ABCF1 negatively regulates type II interferon in TLR2 pathway, whereas positively regulates them in TLR5 and 9 signaling.

Upon viral infection in BMDM ABCF1 was found to be critically important for production of ISG specific proteins like IFNβ and CXCL10 (FIG. 2A). Through TLR3 signaling after poly I:C stimulation, ABCF1 is necessary for STAT 2, 3, 5 and 6 phosphorylation and negatively regulates MAPK phosphorylation (FIG. 2B). Protein analysis in Abcf1 siRNA treated BMDM showed increased NF-KB p65 phosphorylation upon poly I:C stimulation in the nucleus (FIG. 2C), confirming activation of MAPK pathway. Decreased levels of IRF3 phosphorylation and dimerization was seen in Abcf1 siRNA treated and poly I:C stimulated BMDM thus confirming that ABCF1 positively regulates STAT protein activity (FIG. 1B).

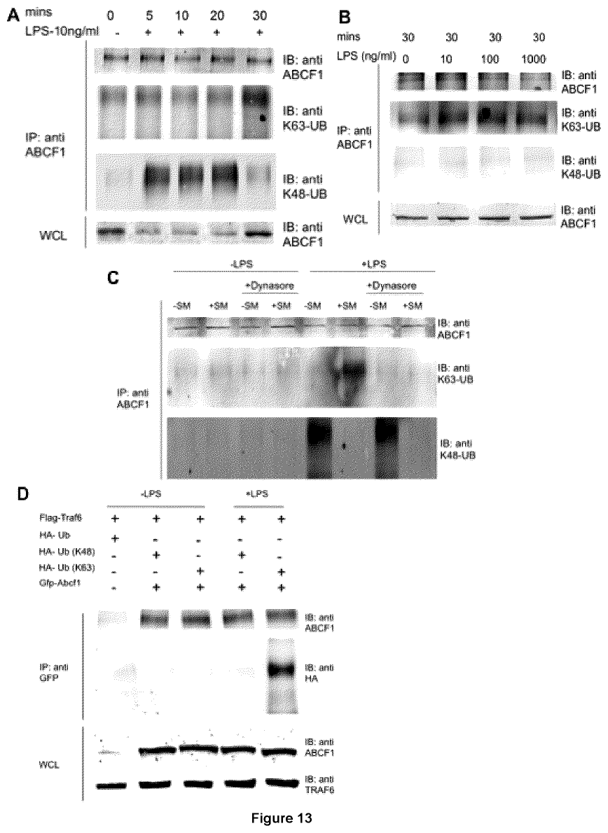

Previous studies have shown that cell counteracts dsRNA infection via Oas1-RNaseL mediated viral degradation (Kerr et al 1978, Kuhn et al 2005, Chakrabarti et al 2011]. We also discovered that ABCF1 regulates this signaling pathway by interacting with OAS1a and controlling its 2′5′A activity. Immuno-precipitation experiments showed that upon poly I:C stimulation OAS1a was immunoprecipitated with ABCF1 (FIG. 3). Protein analysis also showed that ABCF1 positively regulates OAS1a levels in Abcf1 siRNA treated and poly I:C stimulated BMDM (FIG. 13). ABCF1 was also found to negatively regulate protein levels of RNaseL inhibitor, ABCF1, thus positively regulating RNaseL levels (FIG. 3).

### Example 2: ABCF1 Controls TLR4 Endocytosis Through its E2 Conjugating Activity

Here, it is described that ABCF1 functions as an E2 ubiquitin conjugating enzyme, a novel activity amongst ABC transporter genes, regulates TLR4 endocytosis and controls sepsis mediated ET phase by ubiquitinating keys elements involved in the regulation and control of cytokine secretion.

### Results

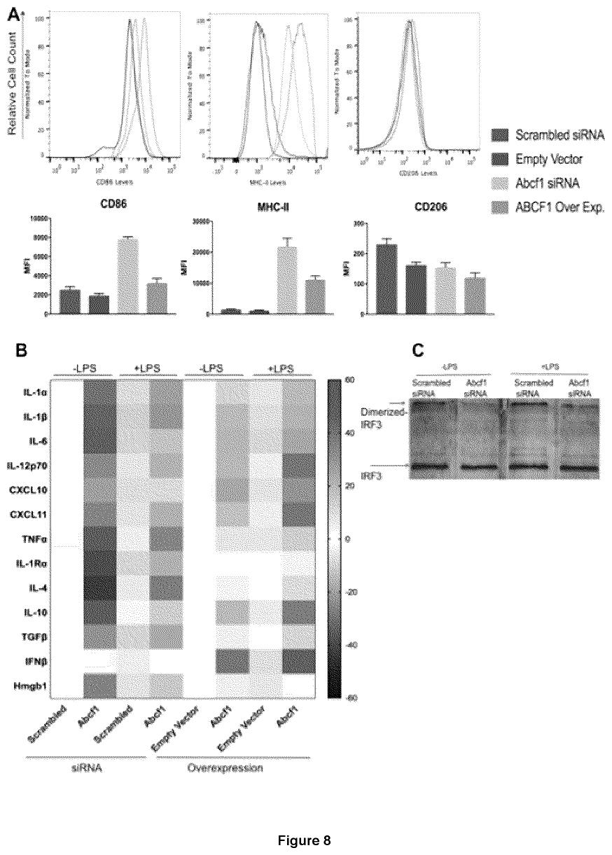

ABCF1 shifts macrophage polarization to M2b phenotype: To investigate if ABCF1 regulates the switch in macrophage polarization, levels of M1 and M2 specific cell surface markers were analyzed with flow cytometry in Abcf1 siRNA treated and over-expressed BMDM stimulated with LPS. Levels of M1 specific markers like CD86 and MHC-II were highest in Abcf1 siRNA treated cells followed by cells overexpressed with ABCF1 (FIG. 8A). On the other hand, M2a specific CD206 levels in both Abcf1 siRNA treated and ABCF1 over expressing cells were either same or marginally less than their respective controls.

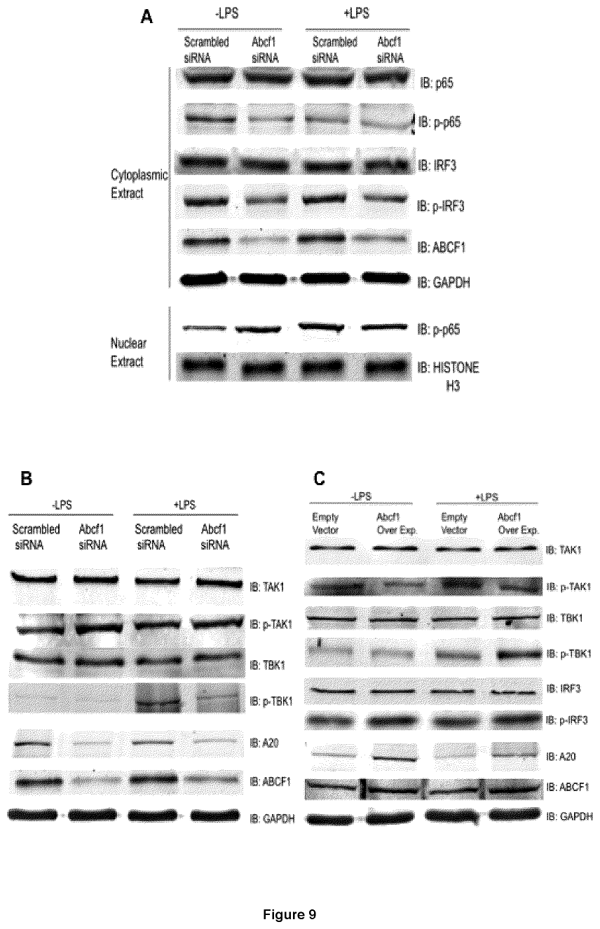

Activation of different transcription factors has also been shown to regulate and differentiate between M1 and M2 specific cell types. Elevated phosphorylated levels of M1 specific NF-KB p65 were observed in the nuclear extracts from Abcf1 siRNA treated cells with and without LPS (FIG. 9A), whereas reduced M2 specific IRF3 phosphorylation and dimerization (FIG. 8C, 9A) was observed in Abcf1 siRNA treated cells with and without LPS.

Additionally, when BMDM were treated with Abcf1 siRNA only, 20 to 40-fold elevation in M1 specific pro-inflammatory cytokines (IL-1β, IL-6, IL-12p70, TNFα) (FIG. 8B) was observed. This up-regulation of pro-inflammatory cytokines was less profound in cells treated with both Abcf1 siRNA and LPS. Conversely, M2 specific anti-inflammatory cytokines (IL-1Rα, IL-4, IL-10, TGFβ) were 20 to 30-fold downregulated in Abcf1 siRNA treated BMDM, both with and without LPS.

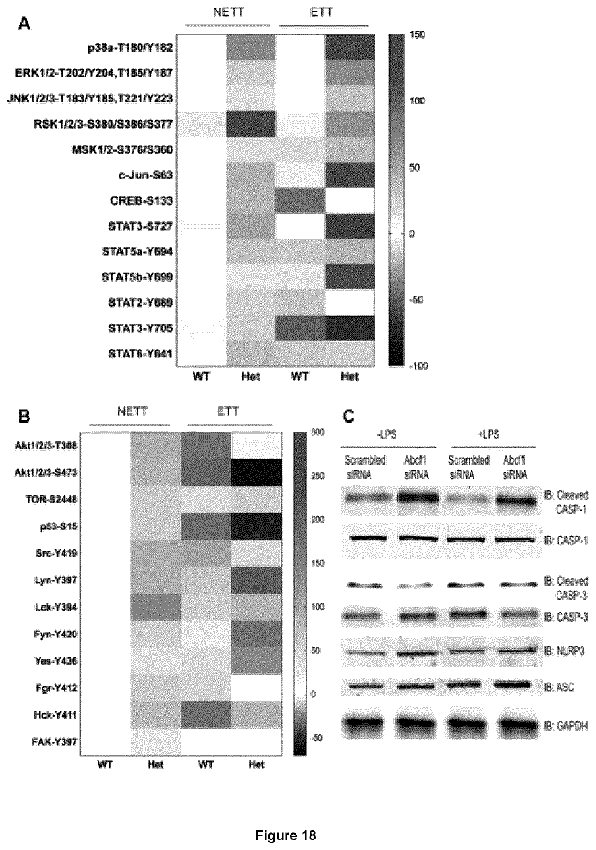

A more moderate trend was seen in supernatants from ABCF1 overexpressing BMDM. The M1 specific pro-inflammatory cytokines showed 5 to 7-fold upregulation with ABCF1 overexpression alone, and were further increased when stimulated with LPS, but their levels were never as high as the ones in Abcf1 siRNA treated supernatants. Whereas M2 specific anti-inflammatory cytokines like (IL-1Rα, IL-4, and TGFβ showed 5 to 10-fold considerable reduction with ABCF1 overexpression alone and continued to decrease after LPS stimulation. However, IL-10 and IFNβ levels were high regardless of LPS and the levels became more profound when treated with LPS (FIG. 18B).

M2a specific macrophages are generally low in pro-inflammatory cytokines and high in anti-inflammatory cytokines with lower MHC-II levels. But here the trend shifted more towards M2b phenotype. M2b specific cells have been shown to produce considerably high IL-1β, IL-6 and TNFα with higher MHC-II and CD86. They produce less IL-4, which is a characteristic cytokine for M2a cells, but more IL-10 and IFNβ. This implies that ABCF1 shifts macrophages to M2b phenotype, and its loss reverts them towards M1.

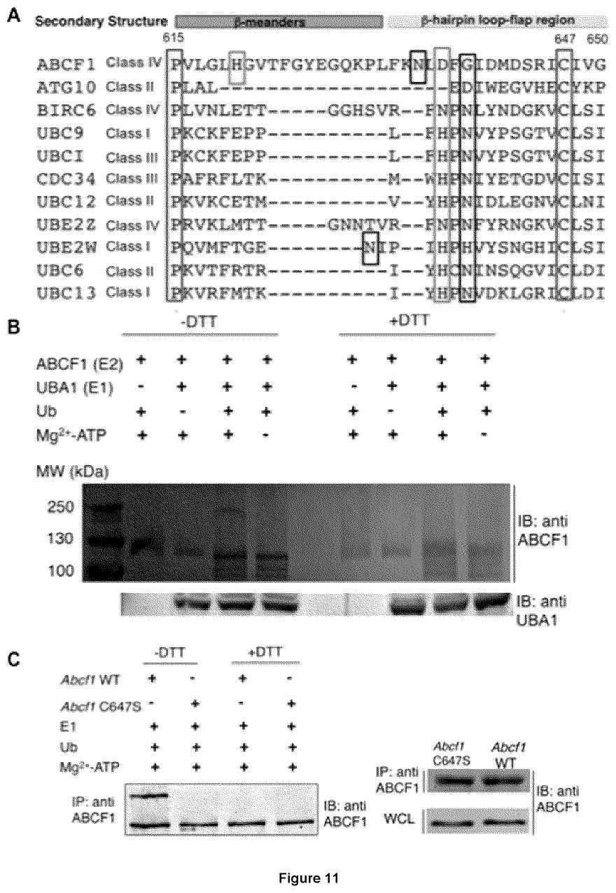

ABCF1 is a novel class IV E2 conjugating enzyme: Sequence alignment of protein sequences from mouse ABCF1 and other known mouse E2 enzymes was performed using CLUSTALW server. Regions in ABCF1 protein sequence that were highly conserved within the E2-UBC domain especially the active site as well as within other residues critical for ubiquitination activity were identified. A cysteine residue in the ABCF1 sequence at position 647 that perfectly aligned with the cysteine residue in the active site from all the other E2's was identified (FIG. 11A). As the secondary structure of ABCF1 has experimentally not been reported either in X-ray or NMR studies, it was predicted the secondary structure of ABCF1 through homology modeling technique using YASPIN and APSSP server tools.

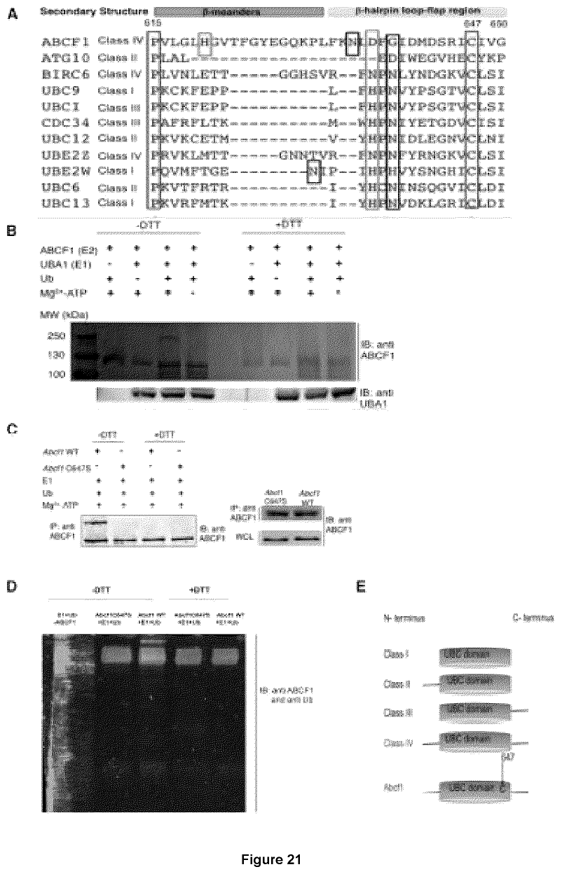

Although the core E2 topology is retained across the UBC domain and the critical catalytic cysteine is shared by all the E2's however, many structural variations have been observed in several members that alter the established structure of E2 active site. ABCF1 displays characteristic β-meanders in its UBC domain followed by formation of a C-terminal active site β-hairpin flap like structure (here after called “flap”) (FIG. 21A) (Burroughs et al 2007). ABCF1 also displayed a well-conserved cysteine residue embedded in the C-terminal region of the flap; as also seen in UBC13 and ATG10 E2's whose structures have earlier been identified (Wu et al 2003). In accordance with previous studies a conserved asparagine in the flap region was observed (FIG. 21A) (here after termed “flap asparagine”), generally found in close proximity (7-15 residues) of the conserved cysteine, Like UBE2W, ABCF1 also displayed the flap asparagine but it was not as well conserved as seen in other E2's. A conserved flap histidine upstream of the flap asparagine has also been observed in the E2 active site. These polar residues have been known to come in contact with the cysteine and help in Ub binding (Burroughs et al 2007). However in ABCF1, presence of other polar residues (arginine, aspartate and asparagine) in the active site and a histidine upstream of the flap region would perhaps assist in Ub binding. Authophagy specific ATG10 on the other hand was seen lacking both flap asparagine and histidine (FIG. 11A) (Burroughs et al 2007). Studies have suggested that both flag asparagine and histidine are only conserved in small number of families and this poor conservation is likely to be a consequence of the differences in target peptide recognized by different E2's (Burroughs et al 2007, Yunus and Lima 2006).

Presence of hydrophobic residues in E2 flap region have been suggested to help in lowering the pKa of the active site and also to mediate E2-E3 binding (Capili et al. 2007). ABCF1 was found to contain most hydrophobic residues among all the E2 shown in (FIG. 21A), which would act to lower the pKa and allow target lysine deprotonation. ABCF1 also displays a well-conserved proline residue, which sits upstream of the flap region and tethers the flap to the β-meander and helps in stabilizing the entire structure (Burroughs et al 2007).

To confirm the finding, enzyme activity of ABCF1 was investigated in presence and absence of cofactor Mg²⁺ and ATP via an in vitro ubiquitination assay. An increase in mass size was seen when recombinant ABCF1, UBA1 (E1 enzyme) and Ub proteins were added in presence of Mg²⁺ and ATP (FIG. 11B). As thio-ester bonds mediate ubiquitin attachment to the E2 enzyme and are DTT sensitive, no such protein band was observed when samples were treated with DTT. This demonstrates the formation of a thio-ester bond between ABCF1 and Ub in presence of E1 and the increase in mass corresponds to the addition of Ub to the ABCF1 cysteine.

To confirm if the conserved cysteine at position 647 in the ABCF1 sequence is the catalytic cysteine that mediates the E2 function, in vitro ubiquitination assay was performed with Abcf1 WT and Abcf1 C647S (where the cysteine was mutated to a serine) protein constructs. No change in mass was seen with Abcf1 C647S, UBA1, Ub in presence of Mg²⁺ and ATP (FIG. 11C).

Earlier studies have described, that the E2 enzymes are divided into 4 classes based on the presence of a N- and/or C-terminus overhangs to the UBC domain. As ABCF1's conserved cysteine is at position 647 and based on bio informatics studies the UBC domain of ABCF1 ends soon after position 647, and as ABCF1 protein is 840 amino acids long, so it has a considerable N-terminus and C-terminus extension, which is a characteristic of class IV E2's Thus, ACF1 is a novel class IV E2 conjugating enzyme with a conserved catalytic cysteine at position 647.

Ubiquitination by ABCF1 is necessary for TLR4 endocytosis: To investigate if ABCF1 is necessary for TLR endocytosis, loss of surface TLR4 levels (a readout of TLR4 endocytosis) on BMDM at various LPS concentrations and time points were analyzed. Flow cytometry analysis of surface TLR4 showed that endocytosis first occurred at as low as LPS 10 ng/ml concentration and as early as 30 minutes (FIG. 12A, B). Surface TLR4 levels were elevated when cells were treated with both Abcf1 siRNA and LPS (FIG. 12C). There was considerably less surface TLR4 levels or (more TLR4 endocytosis) when Abcf1 WT plasmid was overexpressed, but this phenomenon was reversed when the Abcf1 C647S mutant plasmid was over-expressed, suggesting a potential role of ubiquitination by ABCF1 in TLR4 endocytosis. This phenomenon was also confirmed in in vivo mouse experiments. TLR4 levels were analyzed on CD11b+ splenic macrophages from both WT and Het mice. At all timepoints, the ABCF+/− mice showed more TLR4 surface staining, as compared to WT (FIG. 12D), supporting the hypothesis that ABCF1 is necessary with TLR4 endocytosis.

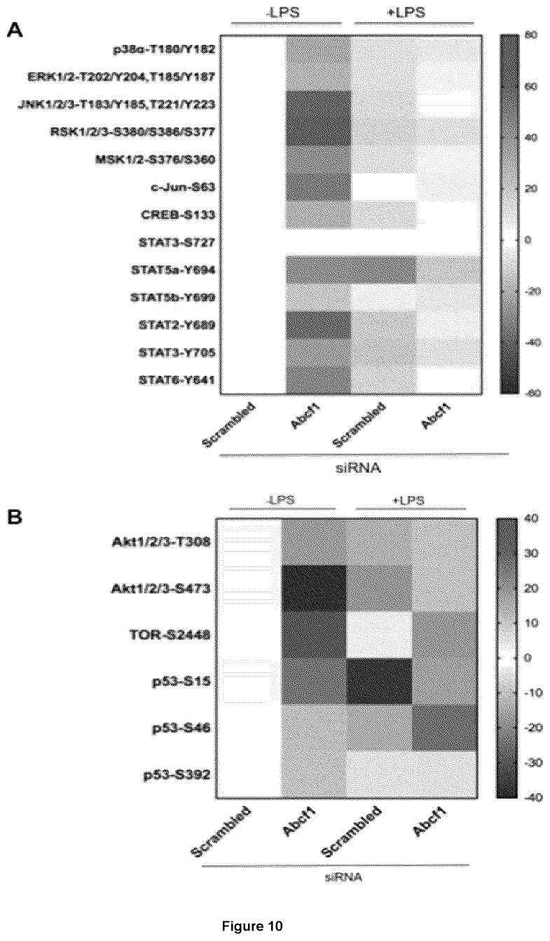

ABCF1 regulates the shift from MyD88 to TRIF dependent signaling: To investigate if the ubiquitination activity of ABCF1 further regulates the shift between the two downstream pathways, phosphorylation levels of various proteins involved in these pathways were analyzed in Abcf1 siRNA treated and LPS stimulated BMDM. Phosphorylation levels of TAK1 were significantly upregulated in cells treated with Abcf1 siRNA only and these levels seemed to have decreased minutely when stimulated with LPS (FIG. 9B). Though LPS stimulation is known to increase phosphorylation of TAK1 and loss of ABCF1 should have complemented it, but in this case after LPS stimulation, TLR4 endocytosis to the endosome, thus triggering the alternate TRIF pathway, which is TAK1 independent (FIG. 8B, FIG. 9B). This was further confirmed when increased p-TBK1 levels were observed in controls treated with LPS only and decreased p-TBK1 levels in cells treated with both Abcf1 siRNA and LPS (FIG. 9B). Levels of A20, a negative regulator of NF-KB pathway increased in controls stimulated with LPS only as compared to Abcf1 siRNA treated cells with and without LPS. Phosphorylated levels of several kinases and MAPK kinase specific transcription factors followed the same trend as for p-TAK1 and interferon stimulating genes specific transcription factors (STAT-2, -3 and -6), followed the same trend as for A20 (FIG. 10A) thus further confirming the hypothesis. This trend was also confirmed by overexpressing ABCF1 in presence and absence of LPS, where levels of p-TAK1, A20, p-TBK1 and p-IRF3 correlated with the TRIF dependent signaling (FIG. 9C).

Ubiquitination ABCF1 regulates TLR4 endocytosis and TRIF signaling through its K63-poly-ubiquitination activity: The data shows that ABCF1 shifts the TLR4 pathway towards TRIF-IFN-I signaling. Investigation showed increased levels of K48-polyubiquitination of ABCF1 when BMDM were stimulated with LPS-10 ng/ml at 5 mins, 10 mins and 20 mins time points, while there was a shift to K63-polyubiquitination of ABCF1 at LPS-10 ng/ml, LPS-100 ng/ml and LPS-1 ug/ml after 30 minutes stimulation (FIG. 13A, B). This suggested that ABCF1 is a target of ubiquitination and is K48-linked and K63-linked poly-ubiquitinated by unknown proteins, which modulates ABCF1's negative regulation of MAPK and NF-KB mediated pro-inflammation at earlier time points and at later time points, modulates ABCF1's role in TLR4 endocytosis.

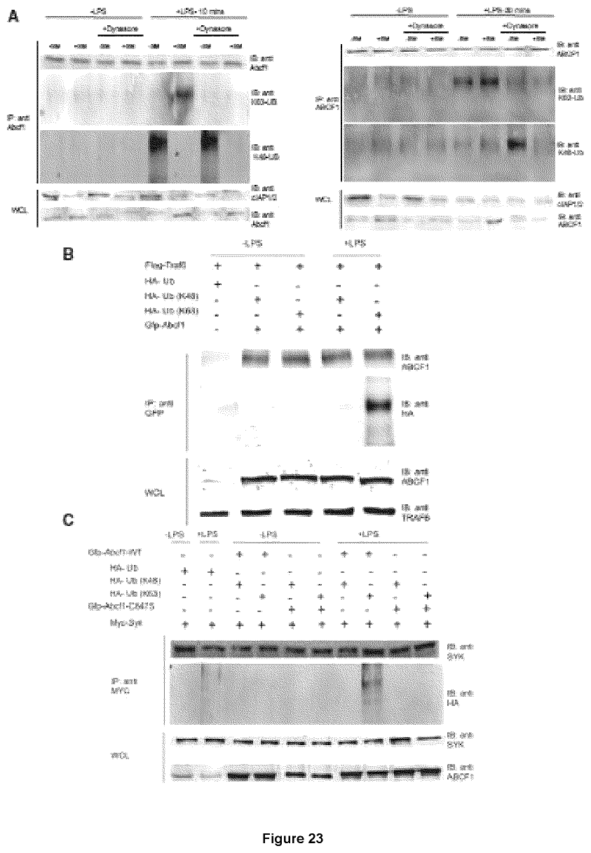

To investigate the possibility of TRAF6 and cIAP1/2 being involved in this signaling, BMDM in presence and absence of LPS were treated with small molecule Smac-mimetic (SM), which triggers cIAP1/2 degradation (Petersen et al 2007) and dynamin inhibitor Dynasore (Dy), which inhibits TLR4 endocytosis (Macia et al 2006). Immunoblot analysis showed that ABCF1 is targeted for K63-polyubiquitination when BMDM were treated with LPS alone and levels of K63-polyubiquitination increased when BMDM were treated with SM in presence of LPS, indicating onset of TLR4 endocytosis mediated TRIF signaling. K63-polyubiquitination of ABCF1 was found to be endocytosis dependent, as no K63-polyubiquitination was observed upon treatment with Dynasore (FIG. 23C). While ABCF1 was targeted for K48-polyubiquitination upon Dynasore treatment, indicating activation of MyD88 signaling and no K48-polyubiquitination of ABCF1 was observed when BMDM were treated with both SM and Dynasore in presence of LPS. This indicates that cIAP1/2 targets ABCF1 for K48-polyubiquitination in HECT E3 domain like fashion in presence of LPS and this step occurs prior to endocytosis.

Elevated ABCF1 protein levels and conversely reduced p-TAK1 levels were observed when BMDM were treated with cIAP1/2 siRNA and LPS (FIG. 14A), confirming cIAP1/2 is essential for TAK1 mediated MAPK, NF-KB signaling and negatively regulates ABCF1 levels.

To further investigate the ubiquitination link between TRAF6 and ABCF1, TRAF6 was co-immunoprecipitated with ABCF1 in BMDM stimulated with LPS confirming a potential role of TRAF6 in ABCF1 mediated signaling (FIG. 14B). Transfection with Traf6, ubiquitin and Abcf1WT plasmids followed by immunoprecipitation with anti GFP and IB with anti HA antibody showed a very faint protein band with HA-K48 transfection regardless of LPS stimulation (FIG. 14D), whereas a very strong protein band was observed when transfected with Flag-Traf6 and HA-K63 plasmids after LPS stimulation. This concluded that upon LPS stimulation, ABCF1 is a target for K63-polyubiquitination by TRAF6 in HECT E3 domain like fashion and this ubiquitination occurs in the absence of cIAP1/2 in endocytosis dependent manner (FIG. 13C).

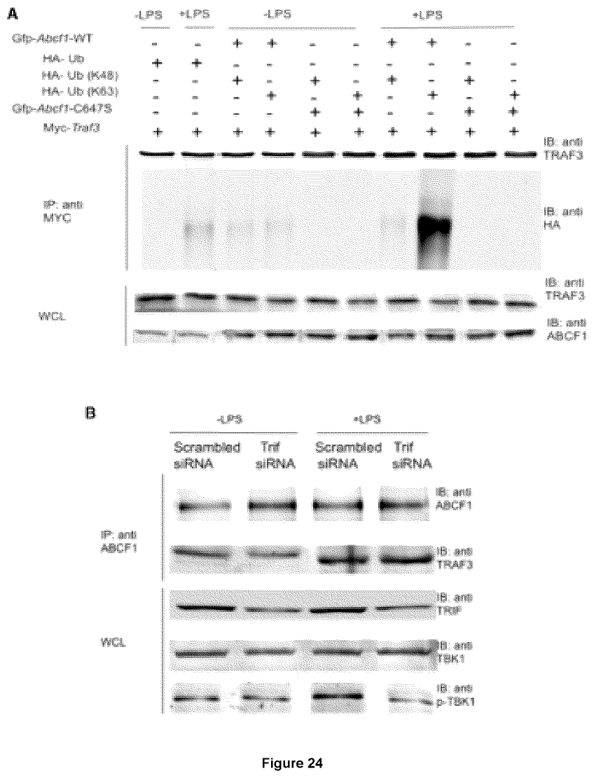

In BMDM, SYK was also found to co-immunoprecipitate with ABCF1 and this was greatly enhanced in BMDM that were stimulated with LPS (FIG. 14B). The overexpression of SYK in BMDM also increased the amount of ABCF1 that could be co-immunoprecipitated with anti-SYK antibody (FIG. 24C). No direct association of ABCF1 was observed with PLCγ2 (data not shown). Loss of ABCF1 was found to be negatively regulating p-SYK and p-PLCγ2 levels (FIG. 14E) in LPS stimulated BMDM, a critical step involved in TLR4 endocytosis. To further investigate the ubiquitination link between ABCF1-SYK and TLR4 endocytosis, plasmids carrying Abcf1, ubiquitin and Syk constructs were transfected in BMDM in presence and absence of LPS and the lysates were immunoprecipitated. Faint to no protein bands were observed with HA-K48 plasmid regardless of LPS, or with HA-K63 in absence of LPS, but strong protein band was observed when co-transfected with Abcf1WT, Syk and HA-K63 plasmids in presence of LPS (FIG. 15A). This protein band totally disappeared when Syk and ubiquitination plasmids were co-transfected with Abcf1 C647S mutation plasmid, suggesting that the conserved cysteine is necessary for ubiquitin transfer and critical for ubiquitinating the target proteins.

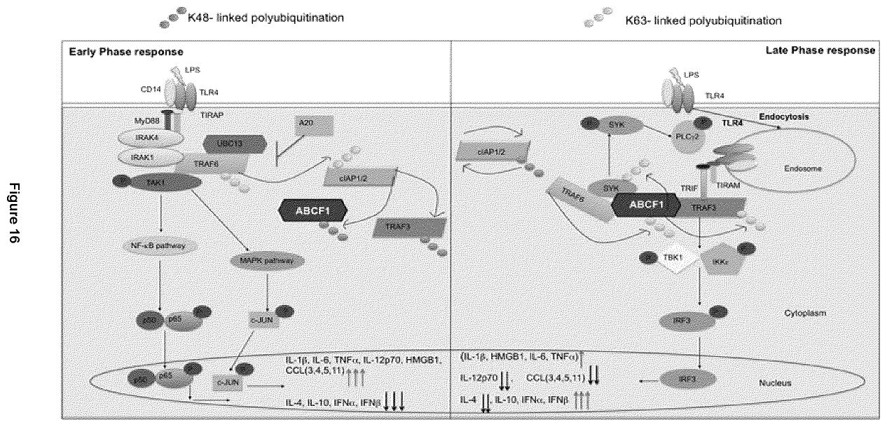

The data shows that TRAF6 and SYK associate with ABCF1 during endocytosis, thus implying formation of TRAF6-ABCF1-SYK complex. This suggests, that after LPS stimulation TRAF6 binds with both target protein SYK and E2 conjugating enzyme ABCF1, acts like a scaffold and aids in K63-polyubiquitination of SYK by ABCF1 in RING domain like fashion (FIG. 16).

ABCF1 targets TRAF3 for K63-poly ubiquitination and regulates IFN-I production: The data thus far indicates that ABCF1 is critical for TLR4 endocytosis, activation and for the phosphorylation of TBK1 and IRF3, and for the production of IFN I. It was also observed that ABCF1 associates with TRAF3, as TRAF3 was co-immunoprecipitated with anti ABCF1 antibody when BMDM were stimulated with LPS and ABCF1 was co-immunoprecipiated with anti TRAF3 antibody when TRAF3 was overexpressed in BMDM (FIG. 14B, D). In order to examine whether ABCF1 targets TRAF3 for ubiquitination instead and modulates the downstream signaling, Abcf1, Traf3 and ubiquitin constructs were transfected in BMDM. Faint to no protein bands were observed with HA-K48 plasmid regardless of LPS, or with HA-K63 in absence of LPS, but strong protein band was observed when co-transfected with Abcf1 WT, myc-Traf3 and HA-K63 in presence of LPS (FIG. 15B).

This implies that upon LPS stimulation, ABCF1 targets TRAF3 for K63-polyubiquitination in a RING domain like fashion. This protein band totally disappeared when plasmids were co-transfected with Abcf1C647S mutant plasmid. No binding was seen between TRAF3 and TRAF6 (data not shown), which E3 ligase aids in this RING domain mediated transfer is still unknown.

Previous studies have shown that TRAF3 ubiquitination is TRIF dependent (Hacker et al 2011, Tseng et al 2010). In order to examine whether ABCF1 is involved in this complex, BMDM was treated with Trif siRNA in presence and absence of LPS, co-immunoprecipitated with ABCF1 antibody and immuno blotted with anti TRAF3 antibody. Strong ABCF1 and TRAF3 binding was observed after LPS stimulation, but no change was observed between scrambled and Trif siRNA (FIG. 15C). On WCL's, Trif siRNA led to decrease in p-TBK1 levels, confirming that TRIF dependent TRAF3 ubiquitination is important for downstream pathway. Thus, it was concluded that TRIF is not necessary for ABCF1 and TRAF3 binding, but perhaps ABCF1 binds with TRAF3 first and then this complex binds to TRIF, via TRAF3, which then initiates ABCF1 mediated K63-polyubiquitination of TRAF3 (FIG. 16).

### Discussion

Endotoxin LPS is the most potent insult in the pathogenesis of sepsis. Sepsis is a bi-phasic inflammatory disease, with the mechanism of shift between its phases still elusive. LPS-TLR4 signaling controls the cytokine storm during sepsis.

ABCF1 controls TLR4 endocytosis, thereby regulating IFN-I response and macrophage polarization, through its K63-polyubiquitination activity. Human ABCF1 also aligns perfectly with other known human E2 sequences and has a conserved cysteine at position 655.

In macrophages, MyD88 dependent early phase of TLR4 signaling leads to UBC13 targeting TRAF6 for K63-polyubiquitination, which further targets cIAP1/2 for K63-polyubiquitination (Hacker et al 2011). cIAP1/2 then enhances K48-mediated proteasomal degradation of ABCF1 (FIG. 13C) and TRAF3 (Tseng et al 2010). In the absence of ABCF1, TAK1 is phosphorylated, which leads to activation of MAPK and NF-KB pathways and elevated production of pro-inflammatory cytokines like TNFα, IL-1β, IL-6, thereby polarizing macrophages to M1 phenotype.

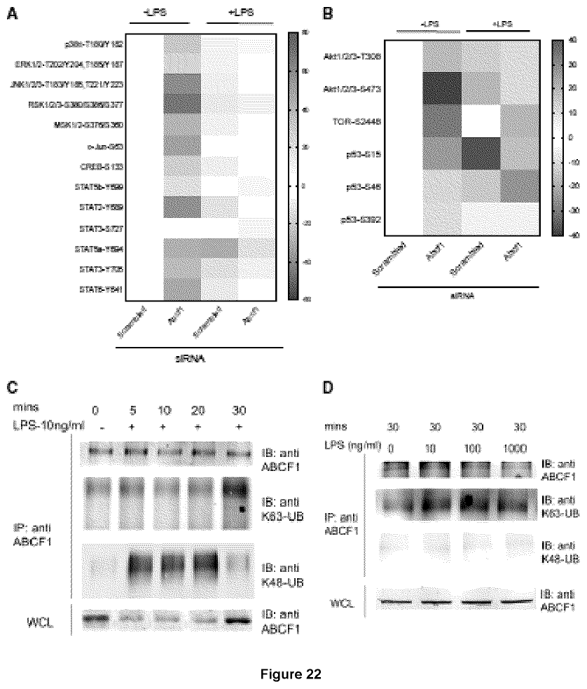

Self K48-proteasomal degradation of cIAP1/2 triggers the onset of late phase TLR4 signaling. Degradation of cIAP1/2 results in activation and K63-linked polyubiquitination of ABCF1 by TRAF6 in a HECT domain like fashion (FIG. 13C, D). Activated ABCF1 then binds and forms a complex with TRAF6 and SYK (FIG. 14B, C, D) and targets SYK for K63-polyubiquitinates in a RING domain like fashion (FIG. 15A). This leads to SYK and subsequent PLCγ2 phosphorylation (FIG. 22, 24E). As both phosphorylation and ubiquitination of SYK by ABCF1 are important for TLR4 endocytosis, further studies are still needed to understand whether these processes work simultaneously or successively. Phosphorylation of SYK and PLCγ2 modulates TLR4 endocytosis into the endosomes, which then initiates TRIF-dependent TLR4 signaling (FIG. 12, 13). This leads to phosphorylation and dimerization of IRF3 and production of IFN-I. ABCF1 was also found to regulate the downstream TRIF signaling by forming a complex with TRAF3 and TRIF and targeting TRAF3 for K63-linked polyubiquitination in TRIF dependent manner (FIG. 15B, C). This triggered phosphorylation of TBK1 that leads to phosphorylation and eventual dimerization of IRF3 (FIG. 8, 15B, C) and production of IFN-I stimulated genes, including IFNβ (FIG. 9A, 16).

This shift regulated by ABCF1's ubiquitination activity not only regulates TLR4 endocytosis, but also controls macrophage polarization. We also discovered that ABCF1 is necessary for increased production of IL-10, minimal production of TNFα, IL-1β, IL-6 (FIG. 8, 9A) and CD86, MHC-II surface markers and decreased CD206 and IL-4 levels, thus polarizing macrophages to M2b phenotype via TRIF dependent signaling (FIG. 16).

### Example 3: ABCF1 Regulates the Shift from SIRS to ET Phase During Sepsis

Sepsis is a bi-phasic disease characterized by initial hyper inflammatory phase also called systemic inflammatory response syndrome (SIRS), which is followed by a latter immuno compromised phase called endotoxin-tolerant (ET) phase. The SIRS phase shows drastic production of pro-inflammatory cytokines like IL1β, TNFα, IL-6 and down regulation of anti-inflammation cytokines like IL-10, IL-4, IL1Rα, whereas the ET phase is completely opposite. In SIRS, the vigorous inflammatory insult generally leads to impaired contractility, reduced cardiac index and ejection fraction, thus leading to circulatory failure. Persistent circulatory failure leads to rupturing of the microcirculation and vasculature thereby leading to multiple organ dysfunction syndrome (MODS), a major determinant of mortality in sepsis. Transition to the ET phase dampens the overall inflammatory response and death in this phase is either due to failure to control primary infection or acquisition of secondary infection.

### Results

ABCF1 targets TRAF3 for K63-polyubiquitination and controls the shift to ET phase during sepsis: To investigate the shift to ET phase during sepsis both WT and Abcf1+/− (Het) mice were treated with endotoxin LPS as described in methods and serum cytokines were measured.

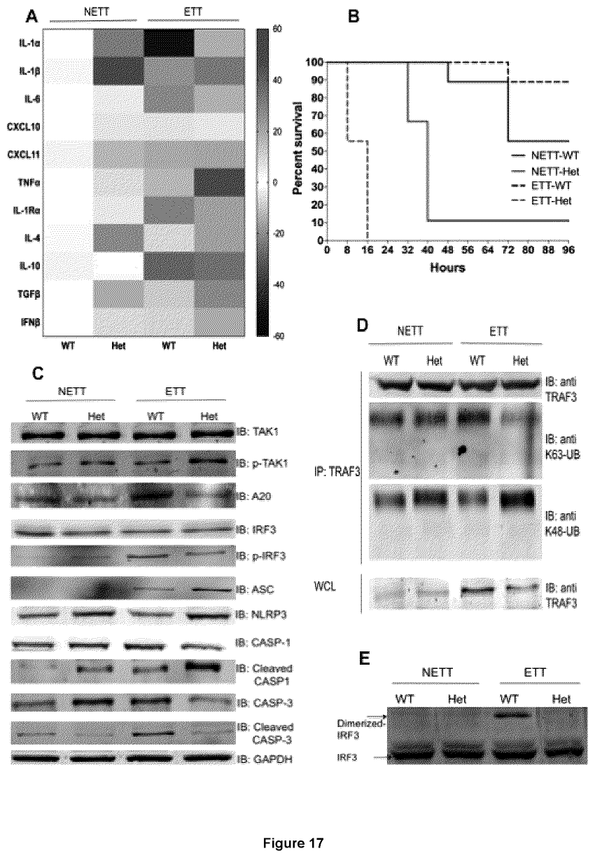

The ETT-WT mice followed the exact same trend of ET phase and showed drastic reduction of IL-1β, IL-6, TNFα, CXCL11 and CXCL10 by 25 to 35-fold. The ETT-Het mice however showed 30-fold increase in IL-6 and 50 to 60-fold elevation in IL-1 and TNFα levels when compared with ETT-WT mice (FIG. 17A), thus exhibiting a phenotype of cytokine storm. Even the anti-inflammatory cytokines in ETT-WT mice followed the regular ET trend, whereas the ETT-Het mice showed 20 to 30-fold downregulation in IL-1Rα, IL-4, TGFβ and IFNβ levels and IL-10 showed even further reduction by 40 to 50-fold.

Survival analysis showed that only 55% ETT-Het mice survived until 8 hours post second injection and there were no survivors after 16 hours. ETT-WT's on the other hand showed 90% survival at the end of 96 hours (FIG. 17B). Protein expression in BMDM from these mice showed that ETT-Het mice had increased p-TAK1 and reduced A20 levels, coupled by low p-IRF3 levels, a characteristic feature of MyD88-dependent signaling during SIRS. On the other hand, BMDM from ETT-WT mice showed reduced p-TAK1, elevated A20 and p-IRF3 levels, a characteristic feature of TRIF-dependent signaling during ET (FIG. 17C).

In order to understand the mechanism of how this shift occurs, immunoblot analysis from BMDM showed increased K48-polyubiquitination of TRAF3 in ETT-Het mice, suggesting K48-mediated proteasomal degradation of TRAF3, hence activating MyD88-SIRS pathway. Increased K63-polyubiquitination of TRAF3 was observed in ETT-WT mice, suggesting activation of TRIF-IFNI-ET pathway (FIG. 17D). Elevated levels of dimerized IRF3 were also observed in ETT-WT BMDM but no IRF3 dimerization was observed in ETT-Het (FIG. 17E). 70 to 100-fold elevation in MAPK levels in ETT-Het mice confirmed inverse regulation of ABCF1 and TRAF3 on inflammation via MAPK pathway (FIG. 18A).

BMDM from ETT-Het mice undergo extensive pyroptosis: Apoptosis via caspase 3 in immune cells in ET phase is the primary cause of acquisition of secondary infection during sepsis, whereas extensive pyroptosis leads to SIRS mediated death during sepsis (Jorgensen and Miao 2015). Pyroptosis is a caspase1-dependent cell death pathway triggered in response to intracellular pathogen signals. NOD like receptor NLRP3 associates with adaptor protein ASC and forms an inflammosome complex with caspase 1, leading to cleavage of caspase1 thereby forming an active complex, which leads to production of IL-1β, HMGB1, TNFα and pyroptosis mediated cell death (Guo et al 2015).

Serum cytokines from ETT-Het mice showed a phenotype of cytokine storm with drastic elevation in IL-1β HMGB1, IL-6 and TNFα (FIG. 17A). BMDM isolated from ETT-Het mice also showed increased NLRP3, ASC and cleaved caspase1 protein levels and decreased cleaved caspase 3 levels when compared with ETT-WT mice, suggesting pyroptosis mediated cell death instead of apoptosis (FIG. 17C). Reduced phosphorylation levels of apoptosis specific kinases like AKT in BMDM from ETT-Het mice (FIG. 18B) further confirmed the hypothesis. Interestingly, similar trend was also observed with Abcf1 siRNA treated and LPS stimulated BMDM (FIG. 18C).

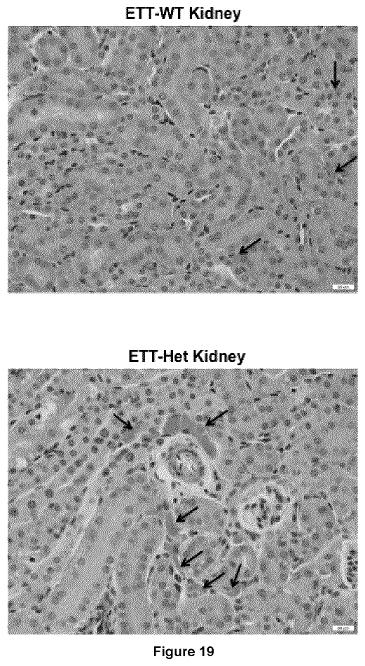

ABCF1 haploinsufficient mice develops Renal Circulatory Failure in SIRS phase: After endotoxin infection ETT-Het mice revert back to SIRS phase and show 50 to 60-fold upregulation of pro-inflammatory cytokines (FIG. 17A). Histology of these mice confirmed the widespread dilation and congestion of small vessels in kidney (FIG. 19). In addition, the images clearly display the sludging effect of red blood cells (shown by black arrows) being compacted together in dilated/congested vessels. This form of vasculature dilation on a systemic scale leads to a critical drop in blood pressure causing insufficient blood and oxygen to reach critical central organs including the brain and heart. ETT-Het mice show significantly increased sludging and vascular dilation as compared to ETT-WT mice suggesting increased congestion of blood vessels contributing to severe hypotension and increased mortality.

### Discussion

ABCF1 was also found inversely regulating the cytokine storm phase during sepsis. It was observed that ABCF1 targets TRAF3 for K63-linked polyubiquitination during sepsis, which triggered IRF3 phosphorylation, dimerization and production of anti-inflammatory cytokines and IFN-I genes (FIG. 17). This regulated the shift from MyD88-dependent SIRS phase to TRIF-dependent ET phase during sepsis. In the Abcf1 haploinsufficient ETT-Het mice, this shift is inhibited, which results in a SIRS dependent phenotype of cytokine storm, whereas the ETT-WT mice show perfect shift to the ET phase and exhibit significant elevation of anti-inflammatory cytokines and IFN-I genes (FIG. 17A).

Examination of mouse survival showed 0% survival of ETT-Het mice after 16 hours of second LPS injection, whereas ETT-WT mice showed 90% survival even after 96 hours (FIG. 17B). Protein expression form ETT-Het mice BMDM showed significant elevation in levels of p-TAK1 and MAPK's and reduction of A20, STAT3 and STAT6 (FIG. 17C, 18A) protein levels, thus confirming the activation of SIRS phase.

Cell damage and host death were found to occur mainly through the downstream effects of pyroptosis. Extensive immune cell pyroptosis in ETT-Het mice could be detected, where elevated levels of HMGB1, NLRP3, ASC and cleaved caspase1 cleavage and reduced levels of cleaved caspase 3 and AKT1 phosphorylation (FIG. 17, 18) were observed. This confirms that ABCF1 is also necessary for regulating the pathway switch leading to death by apoptosis, rather than pyroptosis, in ET phase of sepsis.

Furthermore, histological analysis after endotoxin infection showed alteration in renal microcirculation. Large intravascular clumping of red blood cells, referred to as sludging was seen in greater numbers in ETT-Het mice. This sludging is triggered by alteration in microcirculation and hemodynamics, which is regulated by elevation of pro-inflammatory cytokines in SIRS phase of sepsis (Ramseyer and Garvin 2013). This leads to hypoperfusion mainly in three main organs: heart, brain and kidney (Vincent et al 2006). Significant increase in hypoperfusion in ETT-Het mice kidneys (FIG. 19), in response to upregulation of pro-inflammatory cytokines and MAPK's characteristic of SIRS phase (FIG. 17A, 18A), but no noticeable increase was seen in heart and brain (data not shown) was shown. The ABCF1 promoter activity showed more than 60% ABCF1 promoter activity in kidney as compared to only 10% in heart and roughly 30% in brain (Wilcox et al 2017), and the present data is consistent with Abcf1 Het mice developing visible changes in the microcirculation of kidneys compared to the heart and brain.

Thus, based on both molecular and histological analysis, it is concluded that ABCF1 controls the production of pro-inflammatory cytokines, modulates renal microcirculation, vasculature and targets TRAF3 for K63-linked polyubiquitination and hence is necessary for the shift from SIRS to ET phase of sepsis thereby modulating sepsis mortality.

In summary, a novel ubiquitin E2 conjugating enzyme, ABCF1, which controls both the shift from MyD88 to TRIF dependent signaling of TLR4 endocytosis, hence polarizing macrophages to M2b phenotype and SIRS to ET transition in sepsis, by K63-polyubiquitinating TRAF3 and causing IRF3 nuclear translocation and dimerization was discovered. During sepsis ABCF1 controls the renal microcirculation and vasculature and modulates SIRS mediated mortality.

### Example 4: The ATP-Binding Cassette Gene ABCF1 Functions as an E2 Ubiquitin-Conjugating Enzyme Controlling Macrophage Polarization to Dampen Lethal Septic Shock

Ubiquitination by ABCF1 shifts the inflammatory profile from an early phase MyD88-dependent to a late phase TRIF-dependent signaling pathway, thereby regulating TLR4 endocytosis and modulating macrophage polarization from M1 to M2 phase. Physiologically, ABCF1 regulates the shift from the inflammatory phase of sepsis to the endotoxin tolerance phase, and modulates cytokine storm and Interferon-β (IFNβ) dependent production by the immunotherapeutic mediator, SIRT1. Consequently, ABCF1 controls sepsis-induced mortality by restrain hypotension-induced renal circulatory dysfunction.

### Mice and Cells

Bone marrow cells were isolated from femur and tibia of C57BL/6 mice and were matured into macrophages as described (Trouplin et al., 2013). Briefly, mouse bone marrow was obtained by flushing the tibia and femur of a C57BL/6 mouse with Roswell Park Memorial Institute medium (RPMI) supplemented with 10% heat-inactivated Fetal Bovine Serum (FBS). Bone marrow cells were cultured in 10 ml of RPMI supplemented with 10% FBS, glutamine, and 30% L929 cell supernatant containing macrophage colony-stimulating factor at an initial density of 10⁶ cells/ml in 100-mm Petri dishes at 37° C. in humidified 5% CO₂ for 6 days. Cells were harvested with cold PBS, washed, resuspended in RPMI supplemented with 10% FBS, and used at a density of 2×10⁶ cells/ml.

Abcf1^{+/-} mice (Het; B6.Cg-Abcf1<Gt(XK097)Byg) mice were generated as previously described (Wilcox et al., 2017) and C57BL/6J (Jackson Laboratories; 000664) mice were used as wild type (WT) controls for in vivo experiments. All in vivo experiments were approved and performed in accordance with The University of British Columbia Animal Care Committee and Canadian Council for Animal Care (CCAC).

### Transfections

All gene silencing in cells were done via RNAi interference using Lipofectamine™ RNAiMAX Transfection Reagent (ThermoFisher Scientific; 11668027) according to manufacturer's instructions and the extent of siRNA silencing was checked by western blot. Mouse Abcf1 siRNA (sc-140760), mouse cIAP2 siRNA (sc-29851), mouse Trif siRNA (sc-106845) generated by Santa Cruz Biotechnology is a proprietary pool of 3 target-specific 19-25 nucleotides siRNAs.

All gene overexpressions were done using Lipofectamine™ 2000 Transfection Reagent (ThermoFisher Scientific; 11668027) according to manufacturer's instructions and the extent of overexpression was checked by western blot.

Mouse gene plasmids: Mouse Abcf1WT expression vector was purchased from GenScript (Clone Id: OMu73202). The Abcf1 C647S mutation and GFP tag was cloned both in Abcf1 WT and Abcf1 C647S by Top Gene Technologies (Montreal, Canada). FLAG-Traf6 was a gift from John Kyriakis (Addgene plasmid #21624). pMSCV-mCherry-Syk was a gift from Hidde Ploegh (Addgene plasmid #50045). Mouse Traf3 expression vector was purchased from Origene (MR225761).

Ubiquitin plasmids: pRK5-HA-Ubiquitin-WT (Addgene plasmid #17608) contains the wild type ubiquitin with all lysines intact, pRK5-HA-Ubiquitin-K48 (Addgene plasmid #17605) and pRK5-HA-Ubiquitin-K63 (Addgene plasmid #17606) were gifts from Ted Dawson, and showed 100% similarity with mouse ubiquitin C gene. Ub-K48 and Ub-K63 plasmids contain K48 and K63 lysines only; all other lysines were mutated to arginines.

### Cytokine Analysis

Cell culture supernatant from 2×10⁷ cells or blood serum were prepared and incubated on nitrocellulose membranes containing different anti-cytokine antibodies printed in duplicates provided with Proteome Profiler Mouse Cytokine Array Kit, Panel A (R&D Systems; ARY006) as previously described (Keskinov et al., 2016) and chemiluminescence intensities were measured. Treatments and incubations were done as indicated in the respective figure legends and cytokine analysis was performed according to manufacturer's instructions. Cytokine expression are depicted as fold change and represented as heatmaps as described below.

### Co-Immunoprecipitation

2×10⁷ cells were lysed with freshly prepared 20 mM Tris HCl pH 8.0, 137 mM NaCl, 1% Nonidet P-40, 2 mM EDTA, 1× Halt protease and phosphatase inhibitor cocktail (ThermoFisher Scientific; 78440) and 20 mM N-Ethylmaleimide (Santa Cruz Biotechnology; sc-202719). Protein A or protein G beads were washed twice with wash buffer comprising 10 mM Tris HCl pH 7.4, 150 mM NaCl, 1% Nonidet P-40, 1 mM EDTA, 1× Halt protease and phosphatase inhibitor cocktail and 20 mM N-Ethylmaleimide, centrifuged at 3,000×g for 2 minutes at 4° C. and incubated with indicated antibody for 4 hours at 4° C. on a rotating shaker. Beads were then washed twice and incubated with the cell lysate overnight at 4° C. Beads were washed and the complex was eluted by acidification using 3×50 μl 0.1 M glycine at pH 2 by incubating the sample for 10 minutes with frequent agitation before gentle centrifugation. The eluate was neutralized by adding an equal volume of Tris HCl pH 8.0.

### Immunoblotting

Cell lysates were prepared as described above. All co-immunoprecipitation samples were prepared without DTT in the sample buffer, unless otherwise mentioned. All other western blot samples were prepared with DTT in the sample buffer. Lysates were electrophoresed and immunoblotted with indicated antibodies.

### Antibodies and Chemicals

Antibodies used for western blots and immunoprecipitation, anti ABCF1 antibody for western blotting (ThermoFisher Scientific; PA5-29955), anti ABCF1 antibody for co-immunoprecipitation (Proteintech; 13950-1-AP), anti I-KB antibody (abcam; ab32518), phospho-I-κB antibody (abcam; ab12135), anti NF-KB p65 (abcam; ab16502), anti phospho-NF-κB p65 (abcam; ab86299), anti cleaved CASP1 antibody (Santa Cruz Biotechnology; sc-514), anti CASP1 antibody (abcam; ab108362), anti cleaved CASP3 (Cell Signaling Technology; 9661), anti CASP3 antibody (abcam; ab13847), anti NLRP3 antibody (R&D Systems; MAB7578), anti ASC antibody (Santa Cruz Biotechnology; sc-514414), anti TAK1 antibody (abcam; ab109526), anti phospho-TAK1 antibody (abcam; ab192443), anti TBK1 antibody (abcam; ab40676), anti phospho-TBK1 antibody (abcam; ab109272), anti IRF3 antibody (Santa Cruz Biotechnology; sc-15991), anti phospho-IRF3 (Cell Signaling Technology; 4947), anti SYK antibody (Santa Cruz Biotechnology; sc-1240), anti phospho-SYK antibody (abcam; ab58575), anti P□2 antibody (Santa Cruz Biotechnology; sc-5283), anti phospho-PLCγ2 antibody (Cell Signaling Technology; 3871), anti A20 antibody (Santa Cruz Biotechnology; sc-69980), anti K48 antibody (Cell Signaling Technology; 4289), anti K63 antibody (Cell Signaling Technology; 5621), anti TRAF6 antibody (Santa Cruz Biotechnology; sc-8409), anti cIAP antibody (Santa Cruz Biotechnology; sc-1869), anti TRIF antibody (abcam; ab13810), anti TRAF3 antibody (Santa Cruz Biotechnology; sc-6933), anti UB antibody (Santa Cruz Biotechnology; sc-8017), anti GAPDH antibody (abcam; ab181602), anti Histone H3 antibody (abcam; ab8580), anti GFP antibody (abcam; ab290), anti MYC antibody (abcam; ab9132), anti mCherry antibody (ThermoFisher Scientific; PA5-34974), anti HA antibody (Santa Cruz Biotechnology; sc-7392).

Antibodies for fluorescence associated cell sorting (FACS), CD86 (BioLegend; 105011), CD206 ((BioLegend; 1417097), MHC-II (BioLegend; 141607), TLR4 (BioLegend; 145406), CD11b (BioLegend; 101223).

Chemicals used, NP-40 (abcam; ab142227), Smac Mimetic (Tocris; 5141), Dynamin inhibitor I Dynasore (Santa Cruz Biotech; sc-202592), Lipopolysaccharide (Santa Cruz Biotech; sc-221855), N-Ethylmaleimide (Santa Cruz Biotech; sc-202719), PMSF solution (Santa Cruz Biotech; sc-482875) and Aprotinin (Santa Cruz Biotech; sc-3595).

### Phospho-Kinase Analysis

Cell lysates from 2×10⁷ cells were prepared as described above. Lysates were incubated on nitrocellulose membranes containing different antibodies printed in duplicates provided with Proteome Profiler Human Phospho-Kinase Array Kit (R&D Systems; ARY003B) and chemiluminescence intensities were measured. This kit can also be used for mouse samples and has previously been tested by (O'Hara and Garcea, 2016, Chen et al., 2010). Treatments and incubations were done as indicated in the respective figure legends and phospho-kinase analysis was performed according to manufacturer's instructions. Phospho-kinase expression are depicted as fold change and represented as heatmaps as described below.

### IRF3 Dimerization Assay

2×10⁷ cells were lysed in a buffer containing 50 mM Tris, pH 8.0, 1% NP40, 150 mM NaCl, 1 mM PMSF and 20 μg/ml of aprotinin, and supplemented with native PAGE sample buffer (125 mM Tris, pH 6.8, and 30% glycerol). The samples were separated by native PAGE and analyzed by immunoblotting.

### Cytoplasmic and Nuclear Fractionation

Bone marrow macrophages were stimulated with indicated treatments and cytoplasmic and nuclear fractionation was performed with NE-PER Nuclear and Cytoplasmic Extraction Reagents (Thermo Scientific; 78833) according to manufacturer's instructions.

In vitro Ubiquitination Assay

Abcf1 WT and Abcf1 C647S expression plasmids were overexpressed in macrophages as described above. Before overexpressing Abcf1 C647S expression plasmid, cells were treated with Abcf1 siRNA, as described above to eliminate endogenous ABCF1 expression. Abcf1 siRNA treated cells were washed with cold PBS twice and then Abcf1 C647S expression plasmid was overexpressed. Cell lysates were prepared and subjected to co-immunoprecipitation with anti ABCF1 antibody. Extent of overexpression and co-immunoprecipitation was checked by western blot. Ubiquitination assay was performed on immunoprecipitated samples with Ubiquitylation Assay Kit (abcam; ab139467) according to manufacturer's instructions. Samples were run on a polyacrylamide gel and blotted with anti ABCF1 and anti UB antibodies.

### Bone Marrow Reconstitution

Recipient mice were sub-lethally irradiated (800-1000 Rads) and then reconstituted with bone marrow from donor mice that have been euthanized by isoflurane and CO2 inhalation. Reconstitution was performed on the same day as irradiation, using intra venous tail vein injections. Bone marrow was flushed as discussed earlier and 2×10⁷ bone marrow cells from the donor mice were reconstituted in recipient mice as previously described (Duran-Struuck and Dysko, 2009). Additionally, the irradiated mice were given 10 mg/ml of ciprofloxacin in their acidified drinking water (changed every 5 days) for the first month after irradiation. Mice were monitored every day for 3 days following irradiation, then decreased to every alternate day for the following two months post-reconstitution. During this time, blood was sampled using the saphenous method, every month to check the extent of reconstitution. A CD45.1 versus CD45.2 flow cytometry was also performed that confirmed more that 95% reconstitution.

Both the WT and the Abcf1^{+/-} Het mice are on CD45.2 background, so these mice were reconstituted with WT CD45.1. The CD45.1 mice have a transgenic mutation that encodes the variant CD45.1 as compared to the CD45.2, and will enable us to decipher between host versus donor bone marrow. CD45.1 mice were purchased from Jackson Laboratories (B6.SJL-Ptprc^{a} Pepc^{b}/BoyJ, Stock #002014). After reconstitution was confirmed, mice underwent the endotoxin experiments as outlined.

### In Vivo Endotoxin Tolerance

All in vivo experiments were approved and were performed in accordance with University of British Columbia Animal Care Committee and the Canadian Council for Animal Care (CCAC). 8-10 weeks old female WT and Abcf1 Het mice were either pre-treated with i.p injections of 0.5 mg/kg LPS or with sterile normal saline. 24 hours later, all mice received an i.p. injection of 20 mg/kg LPS. The former group with two LPS injection was named endotoxin treated (ETT) and the latter group with one PBS and one LPS injection was named non-endotoxin treated (NETT). Mice were either euthanized (by 3% isoflurane in O₂ followed by CO₂ inhalation) 3 hours after the second injection, to obtain blood and organs or were monitored every 8 hours for 4 days for survival. Serum was isolated from blood and cytokines were measured.

### Survival Study

Both WT and Abcf1 Het mice were monitored once a day after the first LPS or PBS injection. After the second LPS injection, mice were monitored every 8 hours and were scored on a (0-3) scale based on severity of behaviour and activity, appearance, grooming and posture, respiratory rate, effort and pattern, rectal temperature, pain and body weight. Score of 3 are considered humane endpoint (were euthanized) and include: no palpable fat over sacroiliac region, severely reduced muscle mass, prominent vertebrae & iliac crests. Immobile, not moving when nudged or picked up; animal cannot right itself if placed on side. Piloerection (>75% of body), matted appearance, unkempt and ungroomed with other severe signs of illness. Constant, severe hunching when sitting and walking and non-responsive to treatment. Gasping or open mouth breathing either in cage or when being weighed; struggling to breathe and unable to move around due to difficulty breathing; noisy breathing (squeaking with effort to breathe), <33° C. for three consecutive measurements one hour apart, constant signs of pain that interfere with normal function despite analgesia administration, animal unresponsive and cold to touch, severe skin tent (>10 seconds).

### Histological Analysis

Mice were first anesthetised by ketamine and xylazine mixture (up to 150 mg/kg body weight ketamine and 15 mg/kg body weight xylazine) administered via i.p. injection and then perfused with sterile PBS at a flow rate of 5 ml/minute for 5 minutes and were sacrificed 3 hours after the second LPS injection. Kidney, brain and heart tissues were fixed in 4% paraformaldehyde for 24 hours, washed in PBS and embedded in paraffin. 5-μm-thick sections were cut and were stained with hematoxylin and eosin (HE). Staining was examined using an Olympus BX51 microscope equipped with a 40× objective. Images were generated using an attached Olympus DP73 camera and the high-performance Olympus cellSens software.

### HeatMaps and Bar Graph Generation and Fold Change Calculation

Heatmaps and Bar graphs were generated in GraphPad prism software (GraphPad PRISM Software, San Diego, Calif.). As indicated, intensity of colours depicts upregulation or downregulation of proteins and p-values are indicated.

Fold change of cytokine and phospho-kinase expression in Abcf1 siRNA-treated BMDMs (with or without PAMP treatment) was calculated by normalizing the mean pixel density with scrambled siRNA-treated BMDMs (with or without PAMP treatment). Fold Change of cytokine and phospho-kinase expression in scrambled siRNA- and PAMP-treated BMDMs was calculated by normalizing the mean pixel density with scrambled siRNA- and non-PAMP treated BMDMs.

Fold change of cytokine and phospho-kinase expression in NETT treated Het mice and ETT treated WT mice were calculated by normalizing the mean pixel density with NETT treated WT mice. Fold change of cytokine and phospho-kinase expression in ETT treated Het mice were calculated by normalizing the mean pixel density with ETT treated WT mice.

### Statistical Analysis

The data are presented as the mean±SD. The means were compared by paired or unpaired student t-tests. P-values <0.05 were considered significant, * p<0.05 ** p<0.01 *** p<0.001 **** p<0.0001. Survival is presented as Kaplan-Meier curves and differences were assessed by the log-rank test in GraphPad prism software. Sample size for each experiment is indicated in the figure legend.

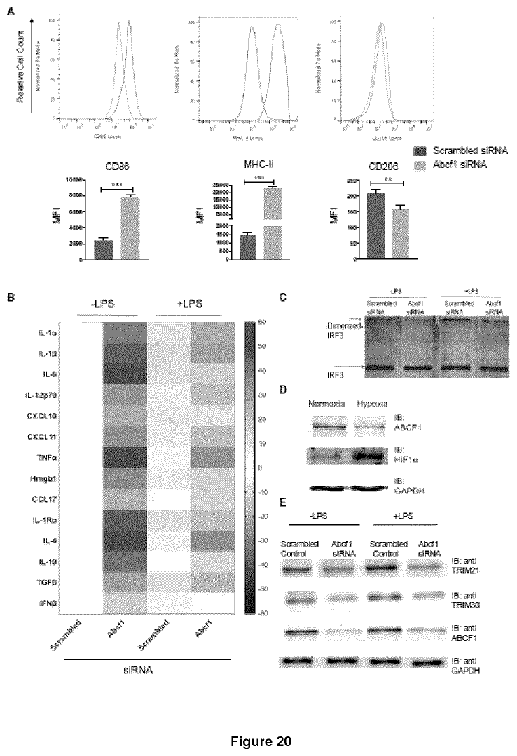

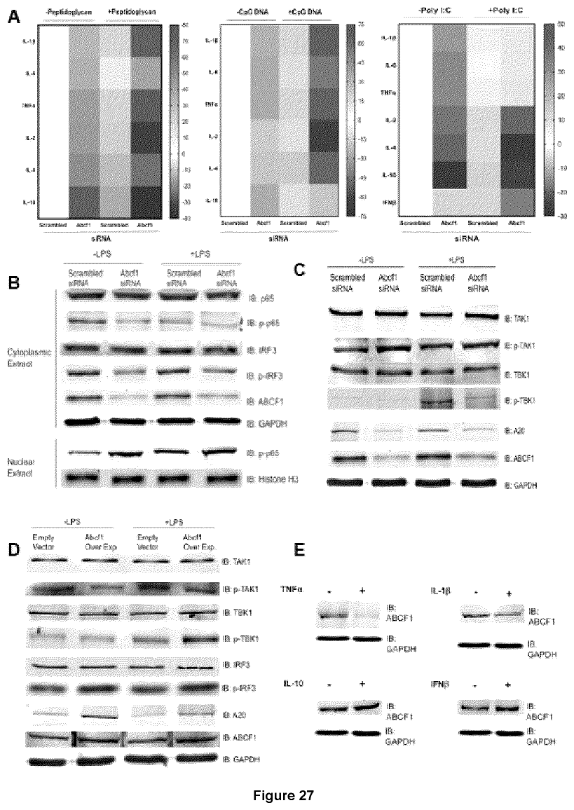

ABCF1 mediates macrophage polarization to the M2 phenotype after LPS stimulation: ABCF1 negatively regulates MyD88-dependent pro-inflammatory cytokine production upon activation of TLR2 (Peptidoglycan) and TLR9 (CpG DNA) signaling and positively regulates TRIF-dependent anti-inflammatory cytokine and IFNβ production upon activation of TLR3 (Poly I:C) signaling in BMDMs (FIG. 27A). Therefore, to investigate if ABCF1 also plays a role in both early and late phase TLR4 signaling and macrophage polarization, the expression of M1 and M2 associated cell surface markers was assessed. Expression of M1 markers like CD86 and MHC-II were elevated in LPS and Abcf1 siRNA-treated cells, when compared with their respective scrambled controls (FIG. 20A). Meanwhile, the M2-specific marker, CD206, was found to be expressed marginally less in LPS and Abcf1 siRNA-treated cells, compared to their respective controls.

Next, whether siRNA silencing of ABCF1 expression in BMDMs would alter the activation status of transcription factors NF-KB (MyD88-dependent) and IRF3 (TRIF-dependent) was examined. Elevated expression of p-NF-KB p65 was observed in the nuclear extracts of Abcf1 siRNA-treated cells, both in the presence and absence of LPS (FIG. 27B), when compared to their respective controls. By contrast, a reduction in IRF3 dimerization (FIG. 20C) and phosphorylation (FIG. 27B) was observed, regardless of LPS stimulation.

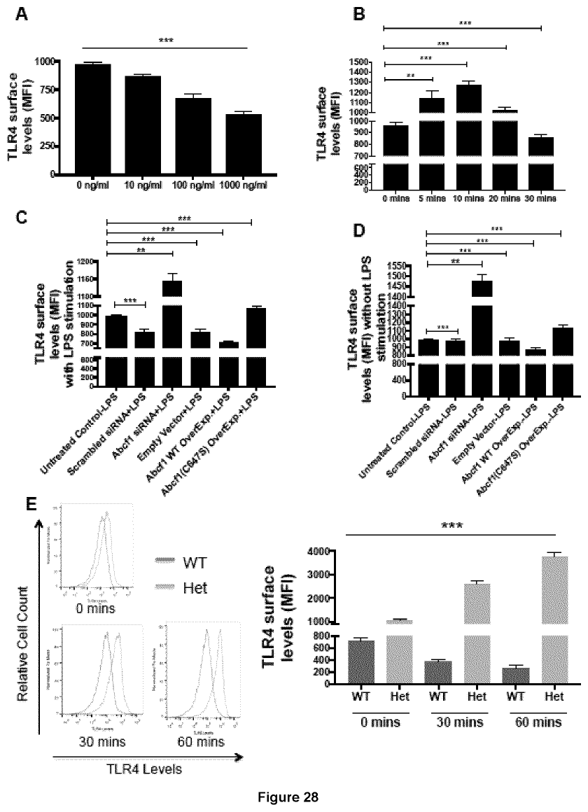

Since macrophage polarization has been tightly linked to the differential expression of various cytokines, the effect of siRNA silencing of ABCF1 on cytokine secretion in BMDMs was assessed. Upon treatment of BMDMs with Abcf1 siRNA alone, a 20 to 40-fold increase in the secretion of M1-associated pro-inflammatory cytokines was observed (FIG. 20B, 28), implying that loss of ABCF1 phenocopies the MyD88-dependent pro-inflammatory signaling. After 30 minutes of LPS stimulation, Abcf1 siRNA-treated BMDMs secreted 30 to 40-fold more of these pro-inflammatory cytokines than LPS treated scrambled controls. Whereas, the decreased expression of M2-associated anti-inflammatory cytokines was observed in Abcf1 siRNA-treated BMDMs, both with and without LPS stimulation (FIGS. 20B, 28).

Upon induction of hypoxia, decreased ABCF1 and increased HIF-1α expression were observed in BMDMs (FIG. 20D) suggesting that loss of ABCF1 is modulated by MyD88-dependent hypoxia. Conversely, immunoblot analysis also showed decreased ABCF1 expression when BMDMs were treated with TNFα and IL-1β whereas increased ABCF1 expression was observed when BMDMs were treated with IL-10 and IFNβ (FIG. 27E). ABCF1 expression correlated with cytokine production after peptidoglycan, CpG DNA, Poly I:C and LPS stimulation (FIG. 20B, 27A). This collectively suggests that ABCF1 expression positively regulates TRIF-dependent anti-inflammatory cytokine and IFNβ production by polarizing macrophages to an M2 phenotype, whereas the loss of ABCF1 phenocopies MyD88 signaling and leads to pro-inflammatory cytokine production by polarizing macrophages to an M1 phenotype.

ABCF1 is a class IV E2 ubiquitin-conjugating enzyme: A screen of Abcf1 siRNA-treated and LPS-stimulated BMDMs identified the regulation of RING tripartite motif (TRIM) E3 ligases (FIG. 20E). Previous studies have shown the involvement of certain TRIM E3s in TLR4 signaling (Kawai and Akira, 2011) and regulation and manipulation of these E3 ligases by E2 ubiquitin-conjugating enzymes (Ye and Rape, 2009). It was hypothesized the possibility of ABCF1 being an E2 Ub-conjugating enzyme, despite the fact that, to date, no E2 ubiquitin-conjugating activity has been reported in the ABC protein family.

The protein sequence of mouse ABCF1 with sequences of known mouse E2 enzymes was aligned using the CLUSTALW server. From this analysis, regions in ABCF1 that are highly conserved within the Ub-conjugating Catalytic (UBC) domain of E2s and are critical for their ubiquitination activity were identified. A cysteine residue at position 647 that perfectly aligns with all the other known E2s in its active site (FIG. 21A). As the secondary structure of ABCF1 has not been experimentally determined, the secondary structure of ABCF1 in silico using the homology modeling tools, YASPIN and APSSP server was predicted.

Although the core E2 topology is retained across the UBC domain and the critical catalytic cysteine is shared by all the E2s however, many structural variations have been observed in several members that deviate from the established structure of an E2 active site, an observation that is not uncommon among E2 enzymes. In agreement with other E2s, homology modeling indicated that ABCF1 contains characteristic β-meanders in its UBC domain followed by a C-terminal active site β-hairpin flap-like structure (hereafter called “flap”) (FIG. 21A) (Burroughs et al., 2008). ABCF1 also possesses a well-conserved cysteine residue embedded in the C-terminal region of the flap; as also seen in UBC13 and ATG10 E2s (Wu et al., 2003). In accordance with previous studies, a conserved asparagine in the flap region (FIG. 21A) (here after termed “flap asparagine”), found in close proximity (7-15 residues) of the conserved cysteine, which has previously been implicated in stabilizing the oxyanion hole in the active site of the enzyme was observed (Burroughs et al., 2008). Like UBE2W, ABCF1 also contains the flap asparagine, but this asparagine is not as well conserved in other E2s. A conserved flap histidine upstream of the flap asparagine is also present in the E2 active site, and these polar residues have been known to come in contact with the catalytic cysteine and promote Ub binding (Burroughs et al., 2008). However, in ABCF1, the presence of other polar residues (arginine, aspartate and asparagine) in the active site and the histidine upstream of the flap region might assist in Ub binding. Alternatively, studies have suggested that both the flag asparagine and histidine residues are only conserved in a small number of families, likely because of differences in target E3 protein recognition (Burroughs et al., 2008). This appears to be the case for ATG10, which lacks both the flap asparagine and histidine residues (FIG. 21A) (Burroughs et al., 2008).

The presence of hydrophobic residues in the E2 flap region is thought to lower the pKa of the active site and mediate E2-E3 binding (Capili and Lima, 2007). ABCF1 was found to contain the greatest number of hydrophobic residues (amounting to ten) among all the E2s shown in FIG. 21A. In theory, this would lower the active site pKa and facilitate target lysine deprotonation. ABCF1 also contains a highly-conserved proline residue, which sits upstream of the flap region and tethers the flap to the β-meander, and helps in stabilizing the entire structure (Burroughs et al., 2008).

To experimentally validate the in silico analysis, in vitro ubiquitination assays to investigate ABCF1 enzymatic activity were preformed, both in the presence and absence of an E1 Ub-activating enzyme (UBA1) and cofactors (Mg²⁺ and ATP). An increase in the apparent molecular weight of ABCF1 was observed when recombinant ABCF1, UBA1, and Ub proteins were incubated in the presence of cofactors (FIG. 21B). The Ub attachment to E2s occurs via thioester linkages and is DTT sensitive, and the observed protein band was lost when samples were treated with DTT. This suggests that a thioester bond was formed between ABCF1 and Ub in the presence of E1 and necessary cofactors, and the increase in apparent molecular weight was consistent with the addition of Ub to the ABCF1 catalytic cysteine.

To confirm that the conserved cysteine at position 647 in the ABCF1 sequence is the catalytic cysteine that mediates the E2 function, in vitro ubiquitination assays were performed with Abcf1 WT and Abcf1 C647S protein constructs. Again, a shift in the apparent molecular weight of ABCF1 was observed when Abcf1 WT construct was used in the assay. Importantly, no such shift was observed with Abcf1 C647S construct, indicating a critical role for this conserved cysteine in the ubiquitination reaction (FIG. 21C).

The Abcf1 WT+E1+Ub lane shows that ABCF1 and ubiquitin merge and display a yellow color, signifying binding of ABCF1 and ubiquitin proteins, which give rise to a second higher protein size band (FIG. 21D). Earlier studies have suggested the categorization of E2 enzymes into 4 classes based on the presence of N- and/or C-terminal extensions to the UBC domain (van Wijk and Timmers, 2010). ABCF1's conserved cysteine is at position 647 and, based on bioinformatics analysis, its UBC domain ends soon after position 647. Considering ABCF1 is 840 amino acids long, it must contain considerable N- and C-terminal extensions, which is characteristic of class IV E2s. Taken together, these data strongly implicate that ABCF1 is a class IV E2 ubiquitin-conjugating enzyme with a conserved catalytic cysteine at position 647.

ABCF1 is necessary for TLR4 endocytosis: Once activated, TLR4 is internalized into endosomes, decreasing its expression on the cell surface (Kagan et al., 2008). This phenomenon activates TRIF-mediated signaling, which leads to the dimerization and nuclear translocation of IRF3, and subsequent production of IFN-I (FIG. 20) (Kagan et al., 2008). As a readout of TLR4 endocytosis, the loss of TLR4 surface expression was monitored over time in BMDMs stimulated with various concentrations of LPS (Zanoni et al., 2011). Flow cytometry analysis of cell-surface TLR4 showed that endocytosis could be observed within 30 minutes of stimulation and at LPS concentrations as low as 10 ng/ml (FIG. 28A, B). Using siRNA to silence ABCF1 in BMDMs, with or without LPS stimulation, resulted in a clear increase in cell-surface TLR4 (FIG. 28C, D). The cell-surface TLR4, in this case, were significantly reduced when cells were stimulated with LPS, as compared to without. After LPS stimulation, TLR4 tends to endocytose in the endosome, but the loss of ABCF1 favors it to be expressed on cell-surface instead (FIG. 28D). Thus, both the Abcf1 siRNA-treated and LPS-stimulated BMDMs do display increased cell-surface TLR4 when compared with their respective controls, but the expression was considerably reduced when compared to their non-LPS treated counterparts (FIG. 28D).

This disparity in cell-surface TLR4 elicited a clear elevation in pro-inflammatory cytokines, NF-KB and MAPK expression in Abcf1 siRNA treated BMDMs in the absence of LPS, when compared with the scrambled controls and LPS treated counterparts. Additionally, both the Abcf1 siRNA and LPS stimulated BMDMs did display an increased pro-inflammatory cytokines, NF-KB and MAPK expression as well, when compared to the scrambled and LPS treated BMDMs, but to a lesser extent, due to comparative reduction of cell-surface TLR4 on these cells than their non-LPS-stimulated counterparts (FIG. 20, 27). Conversely, considerably reduced cell-surface TLR4 was detected in BMDMs engineered to overexpress WT ABCF1. This phenomenon was reversed when BMDMs were modified to overexpress the C647S mutant form of ABCF1, suggesting a potential role for ABCF1-mediated ubiquitination in TLR4 endocytosis (FIG. 28C, D).

These results were confirmed in vivo using Het mice injected with LPS. At every time point examined, CD11b⁺ and F4/80⁺ splenic macrophages exhibited a considerable increase in TLR4 surface staining than their WT counterparts (FIG. 28E).

ABCF1 regulates the transition from MyD88- to TRIF-dependent signaling: The regulation of the shift from MyD88- to TRIF-signaling during TLR4 endocytosis is still an open question in immunology. siRNA silencing of ABCF1 resulted in a modest increase in the p-TAK1 (a MyD88-signaling associated kinase) in BMDMs stimulated with LPS (FIG. 27C). By contrast, phosphorylation of the TRIF-signaling associated kinase, TBK1, was greatly attenuated (FIG. 27C). Reduced expression of A20 (a negative regulator of the MyD88-signaling) in Abcf1 siRNA-treated BMDMs both with and without LPS stimulation was observed. Furthermore, several MyD88-signaling associated kinases like MAPK kinases and MAPK-specific transcription factors followed the same trend of phosphorylation as p-TAK1, while TRIF-dependent production of Interferon Stimulated Genes (ISG)-specific transcription factors followed the same trend of expression as A20 (FIG. 22A, B). Overexpressing ABCF1 in BMDMs corroborated these results, displaying changes in expression of p-TAK1, A20, p-TBK1 and p-IRF3 that were indicative of TRIF-dependent signaling (FIG. 27D). These results support the hypothesis that ABCF1 seems to negatively regulate MyD88-dependent signaling and positively regulates TRIF-dependent signaling in macrophages responding to LPS.

ABCF1 is targeted for K48- and K63-polyubiquitination by cIAP1/2 and TRAF6: Thus far, the data demonstrates that ABCF1 mediates TLR4 endocytosis and facilitates the shift towards the TRIF-IFN-I signaling axis. Since the regulation of TLR4 signaling is known to involve the polyubiquitination of key effectors, including TRAF3, TRAF6, and cIAP1/2 (Kawai and Akira, 2011, Tseng et al., 2010), it was hypothesized that ABCF1 itself might be regulated by polyubiquitination. We observed increased expression of K48-polyubiquitinated ABCF1 when BMDMs were stimulated with LPS (10 ng/ml) for 5, 10, and 20 minutes (FIG. 22C), suggesting that ABCF1 was bound for degradation. It was also observed that polyubiquitination of ABCF1 shifted to K63 linkages after 30 minutes of LPS stimulation (FIG. 22C, D). This suggested that ABCF1 appears to be the target of both K48- and K63-polyubiquitination by other proteins. The timing of the K48- and K63-linkages after LPS stimulation coincided, respectively, with early-phase MyD88-dependent signaling via MAPK and NF-KB pathways at earlier time points, and ABCF1's positive regulation of TLR4 endocytosis and late-phase TRIF-dependent signaling at 30 minutes (FIG. 20, 27, 28).

Better understanding of the regulation of ABCF1 polyubiquitination and the mechanism of the switch from K48 to K63 linkages was sought. cIAP1/2 is an E3 ubiquitin ligase that mediates the formation of K48 linkages on TRAF3 during early-phase TLR4 signaling, a process that inhibits the transition to late-phase TRIF signaling (Hacker et al., 2011, Tseng et al., 2010). Therefore, it was hypothesized that cIAP1/2 might also target ABCF1 for degradation in a similar fashion. Upon stimulation of BMDMs with LPS for 30 minutes, polyubiquitinated forms of ABCF1 were found to be predominantly K63-linked (FIG. 22C, D, 4A). These linkages were even more prevalent when cells were treated with Smac-Mimetic (SM), a small molecule that rapidly triggers cIAP1/2 degradation, in presence of LPS for 30 minutes (FIG. 23A). Inhibiting TLR4 endocytosis with the dynamin inhibitor, Dynasore, greatly reduced the prevalence of K63 linkages and extensively upregulated K48 linkages upon LPS stimulation at 30 minutes. At LPS treatment for 10 minutes, ABCF1 linkages were K48-polyubiquitinated (FIG. 23C, D, 24A), but SM treatment led to K63-polyubiquitination of ABCF1 instead (FIG. 23A). Whereas, at LPS treatment for 30 minutes, cIAP1/2 undergoes self-degradation (Tseng et al., 2010) (FIG. 23A), and SM treatment further enhances the K63-polyubiquitination of ABCF1 (FIG. 23A). Additionally, Dynasore treatment alone also led to K48-polyubiquitination of ABCF1. Whereas, treatment with both SM and Dynasore abolished K48- and K63-polyubiquitination of ABCF1 altogether (FIG. 23A) and (Tseng et al., 2010). These results collectively suggest that cIAP1/2 targets ABCF1 for K48-polyubiquitination upon LPS-TLR4 engagement and this step occurs prior to TLR4 endocytosis.

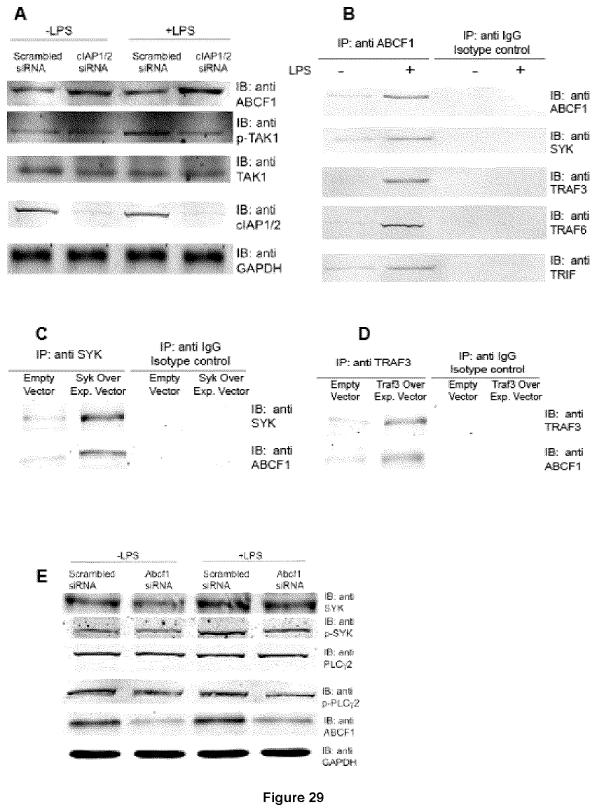

Consistent with this, it was observed that siRNA silencing of cIAP1/2 increased ABCF1 expression and reduced p-TAK1 in BMDMs stimulated with LPS for 30 minutes (FIG. 29A). The observed reduction of p-TAK1 is in line with previous findings that cIAP1/2 is essential for TAK1-mediated MAPK and NF-KB signaling (Tseng et al., 2010) and data of ABCF1's negative regulation of TAK1-mediated MAPK and NF-KB signaling (FIG. 20, 27).

TRAF6 is an E3 ligase that mediates the K63-polyubiquitination of both cIAP1/2 and TRIF (Sasai et al., 2010). It was asked if TRAF6 might also be necessary for the observed formation of K63-polyubiquitin chains on ABCF1 during late-phase (TRIF-dependent) TLR4 signaling. TRAF6 was found to co-immunoprecipitate with ABCF1 in BMDMs stimulated with LPS for 30 minutes, suggesting a potential role for TRAF6 in ABCF1-mediated signaling (FIG. 29B). Next, an in vitro ubiquitination assay was performed where various constructs were co-transfected in BMDMs. After 30 minutes of LPS stimulation, overexpression of TRAF6 was able to strongly induce K63-polyubiquitination of ABCF1, while no K48 linkages were ever detected (FIG. 23B). These findings indicate that early-phase TLR4 signaling targets ABCF1 for cIAP1/2-mediated K48-polyubiquitination, while ABCF1 is K63-polyubiquitinated during late-phase signaling by TRAF6.

ABCF1 associates with and mediates the K63-polyubiquitination of SYK and TRAF3: The study that ABCF1 is an E2-Ub conjugating enzyme prompted to explore whether this activity plays a role in ABCF1's positive regulation of TRIF-dependent signaling. Co-immunoprecipitation studies in LPS-stimulated BMDMs revealed that while ABCF1 associates with SYK (FIG. 29B, C), no interaction with PLCγ2 was observed (data not shown). Further siRNA silencing of ABCF1 was found to decrease p-SYK and p-PLCγ2 expression in LPS-stimulated BMDMs (FIG. 29E). This suggests a mechanism for the previously observed TLR4 endocytosis defect when ABCF1 is silenced.

To investigate if ABCF1's E2 activity regulates SYK, the formation of polyubiquitinated species of SYK in BMDMs co-transfected with various constructs was analyzed. In the absence of LPS stimulation, polyubiquitination of SYK was never detected (FIG. 23C). After 30 minutes of LPS stimulation, K63-polyubiquitination of SYK was detected in BMDMs overexpressing Abcf1 WT, Ub-K63, and Syk plasmids, whereas no K48-polyubiquitin chains were observed. Notably, the formation of these K63 linkages was inhibited when Abcf1 WT was substituted with the Abcf1 C647S mutant plasmid, suggesting that the conserved cysteine is necessary for ubiquitin transfer and critical for the E2 activity of ABCF1.

Previous studies have shown that SYK associates with TRAF6 (Lin et al., 2013) and the data showed that TRAF6 and SYK associate with ABCF1 during TLR4 endocytosis (FIG. 29B, C), possibly implying the formation of a TRAF6-ABCF1-SYK complex. This suggests that during late-phase TLR4 signaling, TRAF6 binds with both target protein, SYK, and E2 enzyme, ABCF1, and perhaps acts like a scaffold to aid in the K63-polyubiquitination of SYK by ABCF1 in an E3-RING domain-like fashion.

Polyubiquitination of TRAF3 is known to be an essential checkpoint in TLR4-TRIF pathway, and non-canonical self-polyubiquitination of TRAF3 is thought to be the key driver for this regulation (Hacker et al., 2011, Tseng et al., 2010). In co-immunoprecipitation studies, an interaction between ABCF1 and TRAF3 was observed (FIG. 29B, D). It is possible that this interaction is indicative of a scenario where the E2 ubiquitin-conjugating enzyme (ABCF1) interacts with an unknown E3 ligase to facilitate the transfer of its ubiquitin moiety to a target substrate (TRAF3) instead.

In order to examine whether ABCF1 targets TRAF3 for polyubiquitination Abcf1, Traf3, and ubiquitin expression constructs were co-transfected in BMDMs. Robust K63-polyubiquitination of TRAF3 were only observed when BMDMs overexpressed Abcf1 WT, Ub-K63, and Traf3 plasmids in the presence of LPS (FIG. 24A). This polyubiquitination reaction required K63 linkages, as negligible amounts of K48 linkages were detected under all other conditions tested. Importantly, K63-polyubiquitination of TRAF3 was completely abolished when the Abcf1 WT plasmid was replaced with the C647S mutant plasmid, indicating a critical role for the conserved catalytic cysteine in this reaction (FIG. 24A). These results suggest that upon LPS stimulation, ABCF1 targets TRAF3 for K63-polyubiquitination in an E3-RING domain like fashion. As no interaction was observed between TRAF3 and TRAF6 (data not shown), the identity of the E3 ligase that aids in this RING domain-mediated transfer is still unknown.

Previous studies have shown that TRAF3 polyubiquitination is dependent on its interaction with TRIF (Hacker et al., 2011, Tseng et al., 2010). To examine whether the interaction between ABCF1 and TRAF3 depends on TRIF, BMDMs were treated with Trif siRNA in the presence or absence of LPS and immunoprecipitated ABCF1. A strong interaction between ABCF1 and TRAF3 was observed after LPS stimulation, and siRNA silencing of TRIF did not affect this association (FIG. 24B). Treating BMDMs with both Trif siRNA and LPS led to decreased p-TBK1, confirming that TRIF-dependent TRAF3 ubiquitination is important for downstream signaling (FIG. 24B) (Hacker et al., 2011, Tseng et al., 2010). Therefore, it appears that although TRIF is not necessary for ABCF1 and TRAF3 association, perhaps ABCF1 first interacts with TRAF3 and then this complex recruits TRIF (FIG. 29), facilitating the ABCF1-mediated K63-polyubiquitination of TRAF3 in a TRIF-dependent manner.

ABCF1 targets TRAF3 for K63-polyubiquitination and controls the IFNβ-SIRT1 dependent sepsis immunotherapy, thereby protecting against sepsis induced mortality: During sepsis, the transition from SIRS phase ET phase is not well understood. Macrophages are thought to be the main producers of inflammatory cytokines during both these phases (Schmauder-Chock et al., 1994, Ge et al., 1997), and their plasticity has been shown to mediate the transition of the disease to the TRIF-dependent ET phase (Biswas et al., 2007, Biswas and Lopez-Collazo, 2009). As ABCF1 governs the switch from MyD88 to TRIF signaling, it is hypothesized that the reduced expression of ABCF1 in Het mice would impair their ability to undergo this transition during gram-negative sepsis.

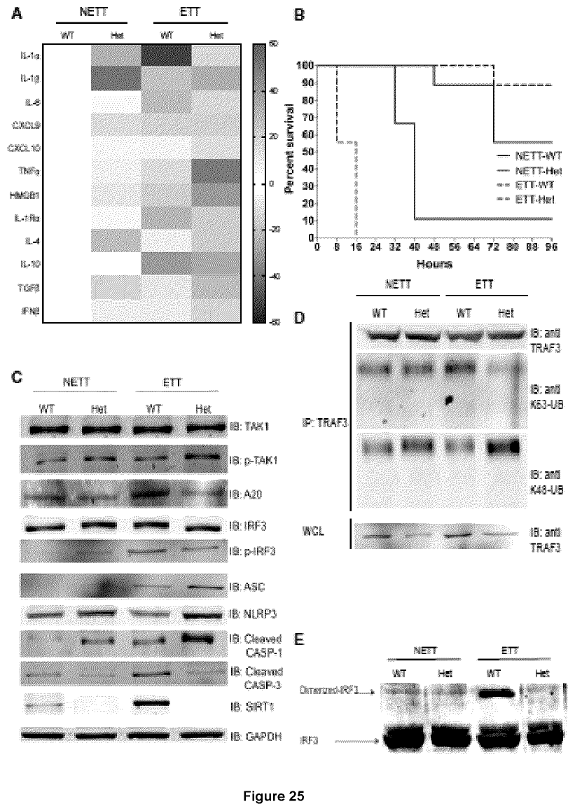

As a model for sepsis, WT and Het mice were injected with endotoxin (LPS). Pretreatment of WT mice with endotoxin (ETT-WT) induced endotoxin tolerance, and resulted in a serum cytokine profile indicative of the ET phase of sepsis. Specifically, it was observed a 30-50 fold reduced expression of pro-inflammatory cytokines, 10-20 fold reduced expression of T cell attracting chemokines (CXCL9 and CXCL10), and 60 fold reduced expression in T cell proliferative cytokine (IL-1α). ETT-WT mice also concomitantly displayed increased expression of anti-inflammatory cytokines and IFNβ, again recapitulating the ET phase of sepsis. In contrast to this, the ETT-Het mice exhibited a 40-60 fold elevation in pro-inflammatory cytokines when compared with ETT-WT mice (FIG. 25A), and a 20-30 fold reduced expression of anti-inflammatory cytokines. The non-endotoxin treated (NETT)-Het mice also exhibited a pro-inflammatory response but to a lesser extent than their ETT-Het counterparts. The serum cytokine profile of ETT-Het mice suggested these mice were unable to transition to the ET phase of sepsis, and remained permanently in the hyper-inflammatory SIRS phase of the disease, thus exhibiting a phenotype of cytokine storm.

The polarized serum cytokine profiles of the ETT-WT and ETT-Het mice were manifested in different survival rates for these mice. Only 55% of ETT-Het mice survived for 8 hours after the second (high-dose LPS) injection, and there were no survivors after 16 hours (FIG. 25B). Yet, ETT-WT mice, had a 90% survival rate by the end of the 96-hour study. By contrast, NETT-WT mice had a 60% survival rate, whereas only 10% of NETT-Het mice survived by the end of 96 hours (FIG. 25B).

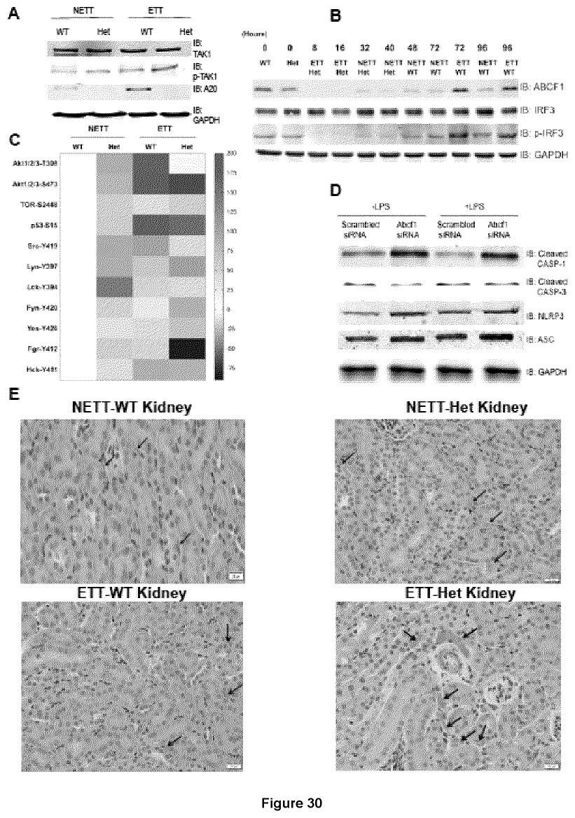

In order to examine innate immune signaling pathways in greater detail, the expression of key kinases and transcription factors signaling in post septic ex vivo cultured splenic macrophages from these mice was analyzed as previously described (den Haan and Kraal, 2012, Deng et al., 2004). Splenic macrophages from ETT-Het mice displayed increased expression of p-TAK1 and reduced expression of A20 and p-IRF3 when compared to ETT-WT (FIG. 30A). Like SPDMs, post septic ex vivo cultured BMDMs have also been shown to regulate sepsis immunotherapy through bone marrow hematopoiesis dependent changes (Muthu et al., 2008). Bone marrow is the center for hematopoiesis, which gives rise to both circulatory (Sunderkotter et al., 2004) and tissue resident macrophages (Davies et al., 2013). It takes 2-5 days for macrophage development from bone marrow progenitors before they transit into circulation. Once in circulation, the immune response elicited by macrophages in peripheral blood around day 2-3 and beyond, are most likely to be a reflection of changes emerging from bone marrow monocytes at an earlier time point (Muthu et al., 2008, Sunderkotter et al., 2004). The ETT-Het mice died within 16 hours of the second LPS injection, as compared to the ETT-WT mice, which showed 90% survival even after 96 hours (FIG. 25B), suggesting that the anomalies seen in these mice were due to changes in the bone marrow and were perhaps ABCF1-dependent. Therefore, it was decided to study the role of BMDMs and bone marrow transplant in sepsis-induced inflammation and mortality. Like splenic macrophages, BMDMs from ETT-Het mice also showed an increase in p-TAK1 and decrease in A20 expression, when compared with ETT-WT mice (FIG. 25C). The predominance of MyD88-dependent signaling in ETT-Het BMDMs and TRIF-dependent signaling in ETT-WT BMDMs mimicked the activity of these signaling pathways during the SIRS and ET phases of sepsis, respectively.

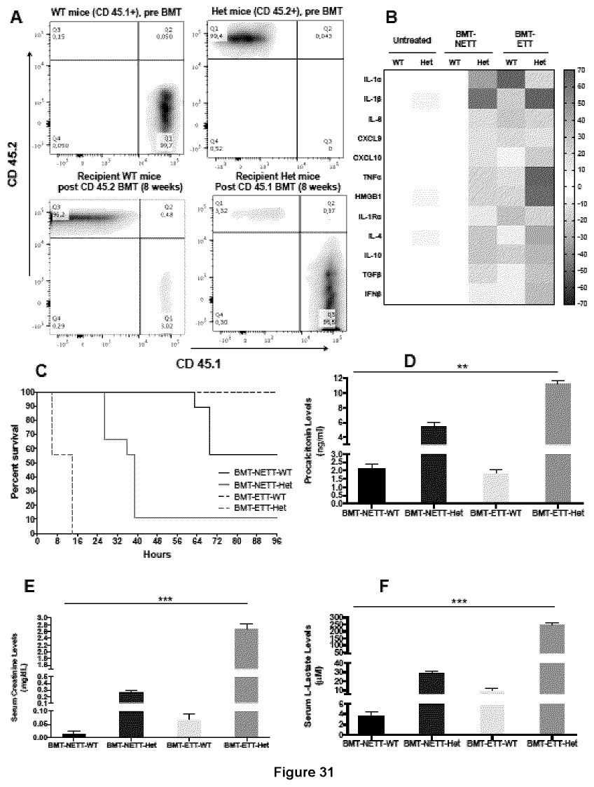

Previous studies have also shown modulation of SIRS phase of sepsis and sepsis-induced mortality by bone marrow transplant (Lorigados et al., 2018, Huang et al., 2017). It was hypothesized that ABCF1 deficiency in bone marrow derived haemopoietic cells was responsible for this increase in susceptibility to developing sepsis. Therefore, a bone marrow transplantation (BMT) experiment where Het bone marrow to WT animals and WT bone marrow to Het animals was reciprocally transferred and sepsis-induced pathogenesis was assessed. The BMT was performed as described and the repopulation of bone marrow was examined (FIG. 31A) and cytokine profiles and mortality were monitored. Even without any endotoxin treatment, Het mice produced increased pro-inflammatory cytokine production when compared to their WT littermates (FIG. 31B). After endotoxin treatment, WT mice that received the Het bone marrow (BMT-ETT-Het) showed the same phenotype of cytokine storm as of ETT-Het mice (FIG. 31B). These mice were more susceptible than regular WT mice to the endotoxin infection and died within 14 hours of second (high dose LPS) injection (FIG. 31C). This timeline was similar to the ETT-Het mice, which died at 16 hours (FIG. 25B). Whereas Het mice that received the WT bone marrow (BMT-ETT-WT) showed the same phenotype of endotoxin tolerance as of ETT-WT mice and showed 100% survival, even after the end of 96 hours (FIG. 31C). These data support the conclusion that ABCF1 genetic deficiency in the haematopoietic lineage transfers susceptibility to endotoxin-induced sepsis, indicating that ABCF1 is a key negative regulator of pathogenesis in sepsis.

Earlier data suggests that ABCF1 is essential for the shift from MyD88- to TRIF-dependent signaling and also for TRAF3-mediated IRF3-dependent IFN-I production (FIG. 24). Therefore, in order to investigate if the immunotherapeutic effect of IFNβ during sepsis depends on the activity of ABCF1, the possibility that ABCF1 targets TRAF3 for ubiquitination during sepsis was examined. IB analysis of ETT-Het BMDMs revealed increased K48-polyubiquitination of TRAF3, suggesting these proteins were destined for proteasomal degradation, hence activating the MyD88 pathway characteristic of SIRS (FIG. 25D). By contrast, increased K63-polyubiquitination of TRAF3 was observed in ETT-WT BMDMs, suggesting activation of the TRIF-IFN-I pathway characteristic of the ET phase (FIG. 25D). Elevated p-IRF3 (FIG. 25C), dimerized IRF3 (FIG. 25E), and IFNβ (FIG. 25A) expression were also observed in ETT-WT BMDMs, whereas no IRF3 dimerization and a 30-40 fold reduction in IFNβ was seen in ETT-Het BMDMs. These results support the hypothesis that ABCF1 seems to be necessary for TRAF3 activation (K63-polyubiquitination) and subsequent IRF3 phosphorylation and dimerization, ultimately leading to the production of IFNβ, a crucially protective cytokine in LPS-induced sepsis.

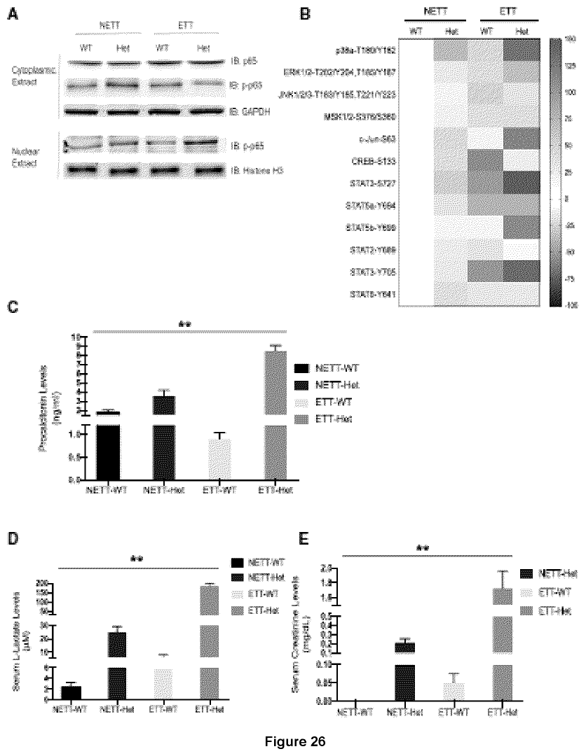

The protective effect of IFNβ during sepsis is thought to depend on its positive regulation of the JAK-STAT pathway and, consequently, SIRT1 production (Yoo et al., 2014). We observed a 50-70 fold reduced expression of p-STAT transcription factors (FIG. 26B) and an absence of SIRT1 in ETT-Het BMDMs (FIG. 25C). Moreover, increased p-NF-KB p65 was observed in BMDMs of ETT-Het mice (FIG. 7A), demonstrating that even a single allele of ABCF1 was insufficient to maintain SIRT1 expression, which thereby regulates inflammation through NF-KB p65. Thus, ABCF1 appears to control the switch from MyD88- to TRIF-dependent signaling during the SIRS-to-ET transition in sepsis, facilitating IFNβ-dependent production of SIRT1.

Physiologically, the expression of ABCF1 in BMDMs from the sepsis-induced mice correlated with their mortality. BMDMs from ETT-Het mice that died 8 and 16 hours after high-dose LPS injection expressed negligible amounts of ABCF1 and p-IRF3 (FIG. 30B). BMDMs from NETT-Het mice exhibited a 90-fold reduced ABCF1 expression and an 85 fold reduced p-IRF3 expression at 32 and 40 hours post high-dose LPS injection, respectively, when compared with untreated WT mice. Additionally, the mortality of NETT-WT mice also correlated with reduced ABCF1 and p-IRF3 expression (FIG. 30B). The ETT-WT group had a 90 fold increased survival rate at the end of the 96-hour study, with only one mouse in the group dying (at 72 hours). BMDMs from this mouse had a 35-fold increase in ABCF1 and 65 fold increase in p-IRF3, when compared with untreated WT BMDMs, but had to be euthanized as it displayed more than 20% weight loss (UBC-CCAC, Star Methods). By contrast, a 47-fold elevation in ABCF1 and 72-fold elevation in p-IRF3 in BMDMs from an ETT-WT mouse that survived the 96-hour study was observed, when compared with the untreated WT group (FIG. 30B). Therefore, ABCF1 expression correlated with both p-IRF3 and mouse survival after high-dose LPS injection and rescued the mice from SIRS-mediated mortality.

BMDMs from ETT-Het mice undergo extensive pyroptosis: While caspase-3-mediated apoptosis of immune cells is the leading cause of secondary infection during the ET phase of sepsis (Hotchkiss et al., 2003), caspase-1-dependent pyroptosis is the primary cause of immune cell death during the SIRS phase of sepsis (Jorgensen and Miao, 2015). Serum cytokines from ETT-Het mice showed a phenotype consistent with cytokine storm, with elevated expression of IL-1β, HMGB1, IL-6 and TNFα (FIG. 25A). BMDMs from ETT-Het mice also showed elevated expression of NLRP3, ASC, and cleaved caspase-1, as well as decreased cleaved caspase-3 when compared with ETT-WT BMDMs (FIG. 25C). In addition, reduced phosphorylation of apoptosis-associated kinases like AKT in BMDMs from ETT-Het mice was observed (FIG. 30C). A similar trend of pyroptosis was also observed in LPS-stimulated BMDMs treated with Abcf1-specific siRNA (FIG. 30D). These results indicate that ABCF1 plays an important role in transitioning BMDMs from pyroptotic to apoptotic cell death pathways during sepsis.

ABCF1 haplo-insufficient mice develop renal circulatory failure in the SIRS phase: Increased expression of circulating pro-inflammatory cytokines is associated with multiple organ failure on the background of persistent circulatory failure (Varpula et al., 2005). During SIRS, endotoxemia leads to the upregulation of TNFα, which alters renal microcirculation and hemodynamics, and leads to acute kidney injury (Ramseyer and Garvin, 2013, Doi, 2016), which ultimately leads to death (Harty, 2014).

ETT-Het mice injected with a high dose of LPS were unable to leave the SIRS phase and showed a 50 to 60-fold upregulation of pro-inflammatory cytokines (FIG. 25A). This was also confirmed by measuring serum Procalcitonin expression. ETT-Het mice displayed significantly increased expression of serum procalcitonin when compared with their WT counterparts (FIG. 26C). Histological analysis of these mice revealed widespread dilation and congestion of small vessels in their kidneys (FIG. 30E). In addition, these images clearly depict the ‘sludging’ effect of red blood cells (shown by black arrows) being compacted together in dilated or congested vessels. This form of vasculature dilation and congestion on a systemic scale results in a critical drop in blood pressure, causing insufficient blood and oxygen to reach critical central organs, including the brain and heart. The kidneys of the ETT-Het mice exhibited a greater sludging and vascular dilation compared to ETT-WT mice, suggesting increased congestion of blood vessels and thus contributing to severe hypotension. ETT-Het mice also displayed an increase in serum L-Lactate expression and serum Creatinine levels (FIG. 26D, E). This suggests that the ETT-Het mice died due to hypertension induced kidney injury due to increased procalcitonin or cytokine storm. Moreover, the bone marrow transplanted mice also displayed the same phenotype, where the BMT-ETT-Het mice showed significantly increased serum procalcitonin, L-Lactate and Creatinine (FIG. 31D, E, F) when compared with their WT counterparts. Collectively this data supports the conclusion that survival during SIRS phase is ABCF1 dependent.

### Discussion

Sepsis is a bi-phasic inflammatory disease, with the molecular mechanisms underlying its immunomodulation and the shift between its SIRS and ET phases still being established. We sought to identify innate immune regulators of LPS-TLR4 signaling and discovered that ABCF1 acts as a molecular switch that mediates the transition from a MyD88-associated cytokine storm phenotype (SIRS) to a TRIF-associated endotoxin tolerant phenotype (ET) during gram-negative sepsis.

Here, the discovery of an E2 ubiquitin-conjugating enzyme, ABCF1, which controls this transition in macrophages is reported. ABCF1-deficient macrophages were defective in TLR4 endocytosis and were unable to transition from MyD88- to TRIF-dependent signaling in response to LPS stimulation. Consequently, they produced predominantly NF-KB- and MAPK-specific pro-inflammatory cytokines, resulting in an M1-like phenotype. Furthermore, during endotoxin-induced sepsis, Het mice were unable to transition from the SIRS phase to the ET phase, displaying exaggerated expression of MyD88-associated pro-inflammatory cytokines in the serum, activated pyroptotic cell death pathways, and finally exhibiting renal circulatory failure which resulted in premature death. These data support the conclusion that physiologically, ABCF1 expression enhances TRIF-dependent anti-inflammatory cytokine and IFN□ production by polarizing macrophages to an M2 phenotype. Inversely, the loss of ABCF1 phenocopies MyD88 signaling and leads to pro-inflammatory cytokine production by polarizing macrophages to an M1 phenotype.

During TLR4 signaling, ABCF1 was able to target SYK for K63-polyubiquitination and also positively regulated SYK and PLCγ2 phosphorylation, which subsequently mediates TLR4 endocytosis. Further studies need to be conducted to determine if this polyubiquitination of SYK is important for its phosphorylation (or vice versa) and subsequent mediation of TLR4 endocytosis. This observation raises the possibility that ABCF1 could also regulate the SYK-mediated endocytosis of other receptors (Dectin-1, FcγRs). As IFN-I is known to be protective during certain viral infections and counteracts cancer progression, the ABCF1-IFN-I signaling axis may be an attractive target for therapeutic intervention.

LPS endotoxin is a potent trigger of gram-negative sepsis. By performing endotoxin-treatment of WT and Het mice following reciprocal bone marrow transplantation, it was established that susceptibility to sepsis is dependent upon the genotype of the haemopoietic compartment and increased resistance to sepsis is dependent on the expression of ABCF1 in the haemopoietic compartment. Thus, ABCF1 normally functions as a dampener of lethal septic shock.

Previous studies have mapped ABCF1 as a risk factor gene for rheumatoid arthritis and autoimmune pancreatitis (Ota et al., 2007, Richard et al., 1998). Additionally, recent genome-wide association studies have also associated ABCF1 with the risk of gout (Dong et al., 2017) and Crohn's disease (Hindorff et al., 2009). The dysregulation of innate immune responses is thought to be a key factor for the prognosis of these immunological disorders, and current research is focusing on modulating cytokine secretion by macrophages. Given that ABCF1 negatively regulates MyD88-dependent TLR2 and TLR9 signaling, positively regulates TRIF-dependent TLR3 signaling, and is essential for the MyD88-to-TRIF transition during TLR4 signaling, it is likely that ABCF1 regulates innate immune responses, macrophage polarization, and cytokine production in these autoimmune disorders. Further work is required to understand the precise role of ABCF1 in the regulation of these diseases.

### Example 5

BACKGROUND: The two major clinically defined forms of inflammatory bowel disease (IBD), Crohn's disease (CD) and ulcerative colitis (UC), are progressive inflammatory conditions that affect the entire gastrointestinal tract and the colonic mucosa, respectively, and are associated with an increased risk for colon cancer. Marked by dysregulation in both innate and adaptive immune regulators, three distinct innate immune CD markers, ATG16L1, NLRP3 NOD2 have been identified. The NLRP3 inflammasome and the related NLR proteins, NOD1 and NOD2, are crucial regulators of inflammatory responses against commensal microflora in the gut. Recently, research has reported higher activation of the NLRP3 inflammasome in patients suffering from Crohn's disease. There is evidence that ABCF1 act as a negative

The effect of diminished ABCF1 levels on the development of Crohn's disease (CD) using an established mouse model: A mouse model of CD where Salmonella typhimurium SL1344 is known to cause a systemic typhi-like disease in mice has been previously described. A model in mice has been established whereby an attenuated aroA mutant (SL1344ΔaroA) that remains localized to the gut endothelium is used to cause a local inflammatory condition similar to CD. Disruption of the local microbiota with antibiotics allows the S. typhimurium to colonize the local gut endothelia to create a chronic infection model in wild type (WT) C57Bl/6J animals (considered ABCF+/+). Previously, MyD88−/− mice were found to have higher numbers of systemic bacteria in their liver and spleen than WT mice, while the numbers found in the colon appeared similar to those found in WT mice, implying that MyD88−/− mice were not as able to contain the bacterial infection. Interestingly, the amount of tissue damage seen in WT mice was found to be more dramatic, with higher goblet cell depletion and damage to epithelial integrity in the colon) and greater fibrosis. In the ABCF1+/− model, it is expected that the decreased amount of ABCF1 in the system will result in a hyper-immune response to the bacterial infection. In the ABCF1+/− mice, more dramatic pro-inflammatory cytokine production and disease severity due to a strong inflammatory response, while seeing a reduction in overall bacterial burden is expected.

The effect of increased ABCF1 levels on the development of CD using an established mouse infection model: In previous studies with LPS-stimulated ABCF1+/− mice, it was found that the lack of ABCF1 resulted in a cytokine storm of inflammation. A similar result upon bacterial infection with S. typhimurium is expected, therefore, whether adding back ABCF1 into the site of infection will be a potential way to ameliorate this condition will be tested. An adenoviral vector expressing the ABCF1 gene will be used to infect the local gut epithelium and some of the infiltrating immune cells in the mouse model, and that the ABCF1 expression will be boosted in these cells. It is expected to calm the inflammatory environment and reduce the amount of tissue pathology seen.

The cellular pathways of ABCF1 in response to S. typhimurium infection: Previous work indicated that serum cytokines from ABCF1+/− mice treated with LPS showed a phenotype of cytokine storm with drastic elevation in IL-1β HMGB1, IL-6 and TNFα levels.

Bone marrow-derived macrophages (BMDM) isolated from these mice also showed increased NLRP3, ASC and cleaved caspase 1 protein levels and decreased cleaved caspase 3 levels when compared with LPS-treated WT mice, suggesting pyroptosis-mediated cell death instead of apoptosis. Pyroptosis (a highly inflammatory form of programmed cell death) and can be initiated by other pathogen-associated molecular patterns (PAMPs) besides LPS. This is relevant to CD as excessive pyroptosis may push the system towards a chronic inflammatory condition. Bone marrow cells from femur and tibia of WT and ABCF1+/− mice that have been previously infected with S. typhimurium (as above) will be isolated. The bone marrow will mature into macrophages as previously described, and the bone marrow-derived macrophages (BMDM) will be studied for their response to bacterial infection. M1 and M2 specific markers, as well as cytokine profiles, will be examined.

The potential benefit of the drug escitalopram in counteracting overt inflammation during CD: S. typhimurium-infected ABCF1+/− mice will be treated with escitalopram in order to increase the expression of ABCF1, and examine for a shift from the exacerbated MyD88 inflammation signaling to TRIF-dependent signaling.

Recent discoveries demonstrate that ABCF1 acts as a molecular switch that negatively regulates the MyD88-dependent proinflammatory signaling pathway by promoting the TRIF-dependent anti-inflammatory signaling pathway that leads to resolution of the inflammatory condition. These pathways lay at the center of the pathogenesis of Inflammatory Bowel Disease. By augmenting expression of known enhancers of the ABCF1 pathway (i.e. escitalopram), new approaches to resolve inflammatory disease and hereby effect wellness in patients living with Crohn's disease may be provided.

### Example 6

A protective role for ABCF1 in a model of septic shock using mice heterozygous for ABCF1 has been defined. However, only 10% of ABCF1(+/−) mice survived 96 hours after high-dose LPS challenge, compared with 60% of wild-type mice. The ABCF1(+/−) mice were unable to switch from the hyperinflammatory phase to the endotoxin tolerance phase, thus suffered exaggerated pro-inflammatory responses and ultimately a renal circulatory failure and premature death. The role of ABCF1 on the development of CD in mice infected with S. typhimurium rather than in the setting of injected LPS alone was assessed. Furthermore, previous work indicated that serum cytokines from ABCF1(+/−) mice treated with LPS showed a phenotype of cytokine storm with drastic elevation in IL-1b HMGB1, IL-6 and TNFα levels. Macrophages isolated from these LPS treated ABCF1(+/−) mice also showed increased NLRP3, ASC and cleaved caspase-1 protein levels and decreased cleaved caspase-3 levels when compared with LPS-treated WT mice, suggesting pyroptosis mediated cell death instead of apoptosis. Pyroptosis (a highly inflammatory form of programmed cell death) can be initiated by other pathogen-associated molecular patterns (PAMPs) beside LPS. This is relevant to CD as excessive pyroptosis may push the system towards a chronic inflammatory condition. Finally, previous work indicated that bone marrow-derived macrophages from ABCF1(+/−) mice polarized towards the pro-inflammatory M1 phenotype, and this was exacerbated by treatment with LPS. The effect of ABCF1, escitalopram and NLRP3 inflammasome inhibitor MCC950 on cytokine production, survival and polarization of macrophages or microglia upon infection with different doses of live attenuated gram-negative bacteria as compared to LPS treatment will be tested.

### Experiments

Bacterial Growth: In the CD model, mice are pretreated with antibiotics and then infected with S. typhimurium strain SL1344DaroA culture (3×106 CFU/mouse) by oral gavage. SL1344DaroA will be grown as previously described, shaken at 370 C (200 rpm) in Luria-Bertani (LB) broth supplemented with 100 μg/ml streptomycin. Both wild-type male and female (WT or ABCF+/+) and male and female ABCF1(+/−) mice will be infected with different doses of the bacterium. Mice will be monitored for the severity of disease for up to 14 days post-infection. Note: all in vivo tests will use the 5 following mouse groups: WT uninfected/healthy; WT infected with SL1344DaroA; and ABCF1(+/−) uninfected/healthy; ABCF1(+/−) infected with SL1344DaroA; and LPS treated ABCF1(+/−) and WT mice as controls. To attempt to reduce pro-inflammatory cytokines, and test if M2 skewing, and survival can be enhanced, a second trial of mice will be orally dosed with escitalopram at concentrations currently approved for use in humans (currently 10-30 mg/human (avg. 62 kg) per day=3.2-9.6 ug/mouse (avg. 20g), or with the NLRP3 inflammasome inhibitor MCC950 injected IP (50 mg/kg) or vehicle control (DMSO/PBS) either 2 weeks pre-injection with LPS or infection or on Day 1 post-injection with LPS or post-infection with S. typhimurium (as above).

Disease severity: Mice will be monitored daily during infection and signs of disease (e.g. weight loss, low temperature, soft or loose stool, absence of grooming behaviour) recorded for a minimum of 7 days. Mice will be euthanized if disease becomes too severe, following animal care guidelines.

Cytokine analysis: Serum will be isolated from blood from above mice on day 3 and day 7, and Creactive protein and cytokines levels will be measured using the Cytometric Bead Array Mouse Inflammation Kit. Serum IL-1β production will be analyzed using the Quantikine Mouse IL-1b/IL-1F2 ELISA kit.

Bacterial counts and histology: Infected mice will be euthanized on post-infection day 3 and day 7, and tissues (colon, liver, brain, spleen) harvested for quantification of S. typhimurium burdens and tissue collection for histology.

Bacterial burden: Collected tissues (colon, liver, brain, spleen) will be weighed and placed into sterile PBS on ice and mechanically homogenized. Serial dilutions of the homogenate will be plated onto LB-agar plates and incubated at 370 C overnight. Bacterial colonies will be counted after 24 hrs.

Histology of inflammation: Analysis of the inflammation taking place within the intestinal environment will be investigated and compared between ABCF1(+/−) and WT mice. Resected colon samples will be taken from mice, both pre- and post-infection with SL1344DaroA, and tissues will be fixed in 10% formalin overnight and then stored in 70% ethanol before paraffin embedding and cutting into 5 μM sections. Tissues will be scored using hematoxylin-eosin-stained sections for the following parameters: polymorphonuclear leukocyte infiltration; goblet cell numbers; epithelial integrity; submucosal oedema.

e. Immune Cell Analysis:
i) Peripheral blood immune cells:

Analysis of the immune cells within the blood will allow us to track the overall numbers and ratios (e.g. CD4/CD8 T cells) and activation status of the cells in ABCF1(+/−) mice versus WT mice. Peripheral blood samples will be taken from mice, both pre- and post-infection with SL1344DaroA, and the overall cellularity will be analyzed using flow cytometry. PBMCs, including lymphocytes, NK cells and monocytes obtained from ABCF1(+/−) and WT littermate controls will be assessed for the expression of markers of activation (e.g. CD69, IAb), memory (e.g. CD62-L, CD44, CD127) and exhaustion (e.g. PD-1, CTLA-4) using flow cytometry. We will determine the total numbers of immune cell populations by staining with specific markers: B cells (e.g. CD19, B220, IgM, IgD, CD20, CD40, CD138 and IAb); CD4+ T cells (e.g. CD4, CD25, CD44 and CD62L); CD8+ T cells (e.g. CD8, CD25, CD44, CD62L, PD-1 and CD127); monocytes (e.g. CD11b, F4/80); and NK cells (e.g. CD335, CD69). Both macrophage and microglia M1 to M2 skewing will be examined using flow cytometry. Proinflammatory and anti-inflammatory cytokine production will be examined.

ii) Histology of inflammation: Colon, liver, lung, brain, and spleen tissues will also be stained for the presence of infiltrating immune cells and cytokine production. Tissue sections will be embedded in Tissue-Tek O.C.T. media (Sakura) on dry ice and immediately stored at −80° C. until sectioning. Ten microns (10 μm) thick sections will be collected on Leica cryostat and fixed in cold acetone or acetone: methanol. Following washing in Tris-buffered saline (TBS, pH 7.4), slides will be incubated with protein block and subsequently with specific antibodies overnight (e.g. T cells: CD4, CD8, FoxP3; B cells: CD19, CD45R, B220; Granulocytes: Ly-6G; Monocytes: CD11b, Mac-1; NK cells: CD335; Cytokines IL-6, IL-1b, TNFa, INFg, INFb, and IL-10). Appropriate horseradish peroxidase (HRP) conjugated secondary antibodies will be used for detection of the primary antibodies and developed with DAB chromogen. Slides will be counterstained with haematoxylin and eosin (H&E) and dehydrated in ethanol and xylene. Giemsa staining will be used to detect eosinophils. Slides will be imaged with an Aperio ScanScope at 20×-40× magnification.

To study role of ABCF1 in MyD88-dependent mediated immune signaling during Crohn's disease in ABCF1(+/−) mice: Macrophages and microglia from SL1344DaroA treated WT and ABCF1(+/−) mice and LPS treated control WT and ABCF1(+/−) mice will be cultured in the presence and absence of escitalopram or MCC950 and western blots using anti-IKB antibodies will be used to assess the levels of NF-KB released into the nucleus by these cells. Macrophages from above mice will also be used to analyze levels of various MAPK's and c-Jun transcription factor. Increased levels of both MAPK's and NF-KB and c-Jun transcription factors will help determine the regulation of onset of MyD88-dependent signaling in CD by ABCF1.

ABCF1 in macrophage polarization during Crohn's disease: Inflammation of the intestinal mucosal epithelium by innate immune regulators is generally governed by M1 macrophages. To investigate if ABCF1 controls this mechanism, levels of M1-specific markers (e.g. CD80, CD86, HCII) and M2-specific makers (e.g. CD206) will be examined in macrophages and microglia from mice flow-cytometry. Elevated levels of M1 specific markers and decreased levels of M2-specific markers in macrophages from SL1344DaroA treated ABCF1(+/−) mice is expected.

Role of ABCF1 in NLRP3 inflammasome regulation during Crohn's disease: It has been shown that in response to LPS in a model of sepsis, ABCF1 negatively regulates NLRP3 inflammasome, ASC and caspase-11 in macrophages. To investigate how ABCF1 regulates NLRP3 during CD, macrophages and microglia from S. typhimurium SL1344DaroA treated WT and ABCF1(+/−) mice will be isolated and levels of the NLRP3 inflammasome, ASC and caspase-1 and assessed via western blot.

Pyroptosis: Different types of cell death can result during bacterial infection, as previously reported where caspase-3-mediated apoptosis of immune cells is prevalent during the endotoxin-tolerant phase of sepsis, however caspase-1-dependent pyroptosis is the primary cause of immune cell death during infection with intracellular bacteria. Therefore, the macrophages and microglia above were analyzed for the expression of proteins associated with apoptosis (CASP-3) and pyroptosis (CASP-1, NLRP3, ASC) by immunoblotting. These experiments will provide evidence as to whether ABCF1 counteracts excessive pyroptosis that augments a chronic inflammatory condition in CD.

### Example 7: The Effect of Diminished ABCF1 Levels on the Development of Disease in Rheumatoid Arthritis (RA)

It is predicted that the depletion of ABCF1 in the ABCF1+/− mouse model will result in a more severe inflammatory condition when these mice are induced with an experimental model of RA compared to control wild-type (WT) mice.

### Experiments

ABCF1 Study Model: To study ABCF1 expression and function in development and disease, ABCF1 knockout mouse model described previously was used.

Induction of RA and disease severity: Cohorts of ABCF1+/− mice and wild type (WT) counterparts will be immunized with type II chick or bovine collagen (CII) and the progression of Collagen-induced arthritis (CIA) will be followed by monitoring joint inflammation by macroscopic examination. Both male and female mice (young: under 3 months, and old: over 8 months) will be studied in the following experiments.

On days 10 to 25 following immunization of mice with 1004 CII and 1004 heat-killed M. tuberculosis, the disease will be evaluated by assessing walking ability, and screening for skin redness and swelling at the site of ankle and wrist joints and small interphalangeal joints to obtain an arthritic index. Using this index, the severity of involvement of each paw can be graded from 0 to 4. Additional monitoring of disease development will involve mouse weight measurements and histological examination of joint tissues. Water displacement will be used to measure increases in hind paw volume, and swelling of the paw will be quantitated by measuring thickness with a thickness-gauge caliper. Mice will be euthanized if disease becomes too severe, in accordance with animal care guidelines. The onset and progression of CIA in ABCF1+/− mice will confirm the involvement of ABCF1 in RA.

Cytokine analysis: Serum (50 μl) and ankle extracts (10 μl) will be isolated from the above mice on day 10 and 25 post-inoculation, and cytokines levels (including: IL-6, IL-10, MCP-1, TNFα, IFNγ, and IL-12) will be measured using the Cytometric Bead Array Mouse Inflammation Kit (Cytometric Bead Array). Serum IL-1β production will be analyzed using the Quantikine Mouse IL-1β/IL-1F2 ELISA kit (Quantikine ELISA). An increase of cytokine production in ABCF1+/− compared to WT mice is expected, suggesting ABCF1 negatively regulates cytokine production.

Histological examination of the infiltrating macrophages in the ankles of CII treated mice. To determine the level of macrophage infiltration in the ankles of CII treated mice, ABCF1+/− mice and their WT controls will be immunized with CII as described above. On day 10 and day 25 post inoculation, mice will be sacrificed and the ankle joints will be paraffin embedded, sectioned and stained with antibodies (including targets: CD11b and F4/80). The number of positive cells (macrophages) from six fields of representative pannus and synovium will be counted under oil emersion at 1000× magnification. The relative number of macrophages in the ankle joints of ABCF1+/− mice and WT controls will be compared and these data may implicate a role for ABCF1 in dampening innate immune responses and inflammation-induced infiltration.

Peripheral blood immune cells: Analysis of the immune cells within the blood will allow us to track the overall numbers, ratios (i.e. CD4/CD8 T cells), and activation status of the cells in ABCF1+/− mice versus WT mice. Peripheral blood samples will be taken from mice, both pre- and post-RA and the overall cellularity will be analyzed using flow cytometry. Peripheral blood mononuclear cells (PBMCs, including lymphocytes, NK cells and monocytes) obtained from ABCF1+/− and WT littermate controls will be assessed for the expression of markers of activation (e.g. CD69, I-Ab), memory (e.g. CD62-L, CD44, CD127), and exhaustion (e.g. PD-1, CTLA-4) using flow cytometry. The composition of the cell population will be determined by staining with lineage markers: B cells (e.g. CD19, B220, IgM, IgD, CD20, CD40, CD138 and MHC-II); CD4+ T cells (e.g. CD4, CD25, CD44 and CD62L); CD8+ T cells (e.g. CD8, CD25, CD44, CD62L, PD-1 and CD127); monocytes (e.g. CD11b, F4/80); and NK cells (CD335, CD69).

Examination of T and B cell responses: Both B and T cell responses will be measured in mice following immunization with CII. As with most antigen systems, the T cell responses against CII peak at around days 10 to 12, and will be monitored until day 25 following immunization. Serum antibody levels peak at a time that coincides with the peak of arthritis severity, and the levels of antibody correlate well with the presence or absence of RA. To examine B cell responses, CII-specific antibody concentrations will be measured using a standard indirect ELISA in which the CII is absorbed to the plate. T cell responses to CII will be evaluated both by proliferation assays and by the production of IFN-β using a solid-phase ELISA. T cell proliferative responses to CII will be measured with a standard microtiter assay using either draining lymph node cells or spleen cells derived from control group or treatment (immunized with full-length CII or CII peptides as antigen, and tritium-labelled thymidine) mice. Additionally, T cell stimulation will be evaluated by measuring IFN-γ production. Several commercial ELISA kits are also available for measuring mouse IFN-γ levels. The examination of T and B cell responses through the monitoring of antibody levels, T cell proliferation, and IFN-γ production in ABCF1+/− mice may provide further evidence that ABCF1 loss of heterogeneity contributes to the onset and progression of CIA.

Potential benefit of the drug, IFNβ-1b, in counteracting overt inflammation during RA: It is known that the ABCF1 gene is substantially upregulated in human synoviocytes following TNFα induction. IFNβ-1b is a cytokine with broad biological activity that is used in hepatitis C and melanoma cancer treatments. It regulates many genes that are involved in antiviral and anti-proliferative activities. It functions by activating the STAT1 transcription factor via the JAK-TYK2 pathway and leads to production of IFN-I via IRF3 and IRF9. This transcription factor then translocates to the nucleus, where it will transcribe several genes involved in cell cycle control, cell differentiation, apoptosis, and immune response. Treatment of RA in ABCF1+/− mice with IFNβ-1b may shift the resulting immune phenotype from a MyD88-mediated hyper-inflammatory response to a protective TRIF-mediated anti-inflammatory response. WT and ABCF1+/− mice will be subcutaneously injected with different concentrations of IFNβ-1b (0.5, 1, 2, 4, 5 μg per mouse—similar to the concentrations used for the treatment of hepatitis), either before CIA induction or one day post-inoculation with collagen. The resulting disease severity, cytokine production, and inflammation will be analyzed as above.

In the ABCF1+/− model, it is expected the decreased expression levels of ABCF1 in the mice will result in a hyper-immune response to RA. In the ABCF1+/− mice, more dramatic pro-inflammatory cytokine production and disease severity due to a strong inflammatory response, while seeing a reduction in overall bacterial burden is expected. Treatment with IFNβ-1b will act to rescue the hyper-inflammatory response in the mice by shifting the pathway from the MyD88-dependent inflammatory signaling to TRIF-dependent signaling (interferon-I production). Treatment with IFNβ-1b before inoculation with CII will allow for testing of whether prophylactic stimulation of ABCF1 expression would be useful as a therapeutic strategy, whereas treatment post-inoculation will allow for testing of the efficacy of the therapy after infection has been established. The fact that other ABC transporters can be modified by drug treatments further suggests that ABCF1 may be a particularly useful target for modifying outcomes in inflammatory processes.

### REFERENCES

Biswas, S. K. et al. 2007. J Immunol, 179, 4083-92.
Biswas, S. K. & Lopez-Collazo, E. 2009. Trends Immunol, 30, 475-87.
Davies, L. C., et al. 2013. Nat Immunol, 14, 986-95.
Den Haan, J. M. & Kraal, G. 2012. J Innate Immun, 4, 437-45.
Deng, J., et al. 2004. Am J Physiol Cell Physiol, 287, C730-6.
Doi, K. 2016. J Intensive Care, 4, 17.
Dong, et al. 2017. Protein Cell, 8, 467-470.
Duran-Struuck, R. & Dysko, R. C. 2009. J Am Assoc Lab Anim Sci, 48, 11-22.
Ge, Y, et al 1997. Relationship of tissue and cellular interleukin-1 and lipopolysaccharide after endotoxemia and bacteremia. J Infect Dis, 176, 1313-21.
Harty, J. 2014. Ulster Med J, 83, 149-57.
Heyman et al. 2012. Nephrol Dial Transplant, 27, 1721-8.
Hindorff, L. A., et al 2009. Proc Natl Acad Sci USA, 106, 9362-7.
Hotchkiss et al. 2003. Scand J Infect Dis, 35, 585-92.
Huang et al. 2017. Cell Death Discov, 3, 16097.
Jantsch et al. 2011. J Leukoc Biol, 90, 551-62.
Keskinov et al. 2016. PLoS One, 11, e0156095.
Lorigados et al. 2018. Shock.
Muthu et al 2008. J Burn Care Res, 29, 12-21.
Nakayama, et al. 2013. Cardiovasc Res, 99, 705-15.
Paytubi, et al. 2008. Biochem J, 409, 223-31.
Ramseyer and Garvin 2013. Am J Physiol Renal Physiol, 304, F1231-42.
Richard et al. 1998. Genomics, 53, 137-45.
Sasai et al. 2010. Mol Immunol, 47, 1283-91.
Sunderkotter et al. 2004. J Immunol, 172, 4410-7.
Varpula et al. 2005. Intensive Care Med, 31, 1066-71.
Wittebole et al. Mediators Inflamm, 2010, 568396.
Wray et al. 2003. Int J Biochem Cell Biol, 35, 698-705.
Wu et al. 2003. EMBO J, 22, 5241-50.
Yoo et al. 2014. Sci Rep, 4, 4220.
Rotin D, Kumar S. Nature reviews Molecular cell biology. 2009; 10(6):398-409.
Deshaies R J, Joazeiro C A. Annu Rev Biochem. 2009; 78:399-434.
Callis J. Arabidopsis Book. 2014; 12:e0174. doi: 10.1199/tab.0174.
Winn P J et al Structure. 2004; 12(9):1563-74.
van Wijk S J, Timmers H T. FASEB J. 2010; 24(4):981-93.
Heaton S M, et al. J Exp Med. 2016; 213(1):1-13.
Chen Z J. Nat Cell Biol. 2005; 7(8):758-65.
Hacker H, et al. Nature reviews Immunology. 2011; 11(7):457-68.
Tseng P H et al. Nature immunology. 2010; 11(1):70-5.
Richard M et al. Genomics. 1998; 53(2):137-45.
Ota M et al. Immunogenetics. 2007; 59(1):45-52.
Lee M N et al. Nature immunology. 2013; 14(2):179-85.
Wilcox S M et al. PLoS One. 2017; 12(5):e0175918.
Trouplin V et al. J Vis Exp. 2013; (81):e50966.
O'Hara S D et al. MBio. 2016; 7(6).
Chen P C et al. The Journal of clinical investigation. 2010; 120(12):4353-65.
Deak M, et al. The EMBO journal. 1998; 17(15):4426-41.
Soloaga A, et al. The EMBO journal. 2003; 22(11):2788-97.
Mashimo T, et al. Genomics. 2003; 82(5):537-52..
Kakuta S, et al. J Interferon Cytokine Res. 2002; 22(9):981-93.
Li G, et al. The Journal of biological chemistry. 2004; 279(2):1123-31.
Malathi K et al. RNA. 2010; 16(11):2108-19.
Ray S et al. J Gastroenterol Hepatol. 2003; 18(4):393-403.
Kekow J, et al. The Journal of clinical investigation. 1991; 87(3):1010-6.
Stylianou E, et al. Clin Exp Immunol. 1999; 116(1):115-20.
Gantier M P, Williams B R. Cytokine Growth Factor Rev. 2007; 18(5-6):363-71.
Chakrabarti A, et al. J Interferon Cytokine Res. 2011; 31(1):49-57.
Sen A, et al. J Virol. 2011; 85(8):3717-32.
Meng H, et al. J Mol Biol. 2012; 422(5):635-49.
Zanoni I et al. Cell. 2011; 147(4):868-80.
Peiser L, et al. Infection and immunity. 2000; 68(4):1953-63.
Garcia-Garcia E, Rosales C. J Leukoc Biol. 2002; 72(6):1092-108.
Komander D. Biochemical Society transactions. 2009; 37(Pt 5):937-53.
Janeway C A, Jr., Medzhitov R. Annu Rev Immunol. 2002; 20:197-216.
Kawai T, Akira S. Arthritis Res Ther. 2005; 7(1):12-9.
Hu H, Sun S C. Cell Res. 2016; 26(4):457-83.
Kagan J C, et al. Nature immunology. 2008; 9(4):361-8.
Tanimura N et al. Biochem Biophys Res Commun. 2008; 368(1):94-9.
Borst P, Elferink R O. Annu Rev Biochem. 2002; 71:537-92.
Janeway C A, Jr. Curr Biol. 1999; 9(9):R342-5.
Kawai T, Akira S. EMBO Mol Med. 2011; 3(9):513-27.
Ye Y, Rape M. Nature reviews Molecular cell biology. 2009; 10(11):755-64.
Burroughs A M et al J Struct Biol. 2008; 162(2):205-18.
Wu P Y et al. The EMBO journal. 2003; 22(19):5241-50.
Yunus A A, Lima C D. Nature structural & molecular biology. 2006; 13(6):491-9.
Capili A D, Lima C D. Curr Opin Struct Biol. 2007; 17(6):726-35.
Petersen S L et al. Cancer Cell. 2007; 12(5):445-56.
Macia E et al. Dev Cell. 2006; 10(6):839-50.
Lin Y C et al. Sci Signal. 2013; 6(289):ra71.
Reinhart K et al. Rev Bras Ter Intensive. 2013; 25(1):3-5.
Wood K A. Pharmacoeconomics. 2004; 22(14):895-906.
Hotchkiss R S. Nature reviews Immunology. 2013; 13(12):862-74.
Cavaillon J M, Adib-Conquy M. Crit Care. 2006; 10(5):233.
Annane D et al. Lancet. 2005; 365(9453):63-78.
Ren J, Wu S. Front Biosci. 2006; 11:15-22.
Levi M, et al. Semin Thromb Hemost. 2010; 36(4):367-77.
Otto G P et al. Crit Care. 2011; 15(4):R183.
Du L et al. Biomed Res Int. 2014; 2014:898646.
Schmauder-Chock E A et al. Histochem J. 1994; 26(2):142-51.
Ge Y et al. J Infect Dis. 1997; 176(5):1313-21. PubMed PMID: 9359733.
Hotchkiss R S et al. Scand J Infect Dis. 2003; 35(9):585-92.
Jorgensen I, Miao E A. Immunol Rev. 2015; 265(1):130-42.
Guo H, et al. Nat Med. 2015; 21(7):677-87.
Vincent J L et al. Crit Care Med. 2006; 34(2):344-53.
Harty J. Ulster Med J. 2014; 83(3):149-57.
Xiong J, et al. Sci Rep. 2015; 5:16724.
Rocha e Silva M. Agents Actions. 1978; 8(1-2):45-9.
Raj T et al. Am J Hum Genet. 2013; 92(4):517-29.
Rossin E J, et al. PLoS Genet. 2011; 7(1):e1001273.
Julia A, et al. Arthritis and rheumatism. 2008; 58(8):2275-86.
Franke A, et al. Nature genetics. 2010; 42(12):1118-25.
Weyand C M et al Annals of internal medicine. 1992; 117(10):801-6.
Gross J. Wound Repair Regen. 1996; 4(2):190-202.
Michalopoulos G K, DeFrances M C. Liver regeneration. Science. 1997; 276(5309):60-6.
Goss R J. Clin Orthop Relat Res. 1970; 69:227-38.
Stocum D L. Differentiation. 1984; 27(1):13-28.
McBrearty B A et al. Proceedings of the National Academy of Sciences of the United States of America. 1998; 95(20):11792-7.
Fuchs Y, Steller H. Nature reviews Molecular cell biology. 2015; 16(6):329-44.
Li Y et al. Genes & development. 2014; 28(23):2597-612.
Anderson K V et al. Cell. 1985; 42(3):791-8.
Rocchi, A. et al. Can J Gastroenterol 26, 811-817 (2012).
Marchiando, A. M. et al. Cell Host Microbe 14, 216-224 (2013).
Kanneganti, et al Immunity 27, 549-559 (2007).
Liu, L. & Li, X. Dig Dis Sci 62, 2211-2214 (2017).
Lazaridis, L. D. et al. Dig Dis Sci 62, 2348-2356 (2017).
Powell, T. R. et al. J Psychopharmacol 27, 609-615 (2013).
Mansson, L. E. et al. Am J Physiol Gastrointest Liver Physiol 303, G311-323 (2012).

1. A method of inhibiting an inflammatory response and/or an immune response in a patient in need thereof, said method comprising administering an agonist of ABCF1.

2. A method of inhibiting an inflammatory response and/or an immune response, said method comprising administering an ABCF1 protein or a polynucleotide encoding ABCF1.

3. The method of claim 2, wherein the ABCF1 protein is a soluble ABCF1 protein.

4. The method of claim 2, wherein the polynucleotide is a vector.

5. The method of claim 4, wherein the vector is a viral vector.

6. The method of any one of claims 1 to 5, wherein the patient is a patient with an autoimmune disease.

7. The method of claim 6, wherein the autoimmune disease is inflammatory bowel disease, rheumatoid arthritis, or pancreatitis.

8. The method of claim 7, wherein the inflammatory bowel disease is Crohn's disease or ulcerative colitis.

9. A method of preventing and/or treating sepsis, said method comprising administering an agonist of ABCF1.

10. A method of preventing and/or treating sepsis, said method comprising administering an ABCF1 protein or a polynucleotide encoding ABCF1.

11. The method of claim 10, wherein the ABCF1 protein is a soluble ABCF1 protein.

12. The method of claim 10, wherein the polynucleotide is a vector.

13. The method of claim 12, wherein the vector is a viral vector.

14. A method of treating an autoimmune disease, said method comprising administering an agonist of ABCF1.

15. A method of treating an autoimmune disease, said method comprising administering an ABCF1 protein or a polynucleotide encoding ABCF1.

16. The method of claim 15, wherein the ABCF1 protein is a soluble ABCF1 protein.

17. The method of claim 15, wherein the polynucleotide is a vector.

18. The method of claim 17, wherein the vector is a viral vector.

19. The method of any one of claims 15 to 18, wherein the autoimmune diseases is inflammatory bowel disease.

20. The method of claim 19 wherein said inflammatory bowel disease is Crohn's disease or ulcerative colitis.

21. A method of determining clinical outcome of diseases and/or disorders associated with increased or decreased inflammatory and/or immune responses, said method comprising determining expression of ABCF1.

22. Use of an agonist of ABCF1 to reduce an inflammatory response and/or an immune response in a patient in need thereof.

23. A method of inhibiting an inflammatory response and/or an immune response, said method comprising administering an ABCF1 protein or a polynucleotide encoding ABCF1.

24. The method of claim 2, wherein the ABCF1 protein is a soluble ABCF1 protein.

25. The method of claim 2, wherein the polynucleotide is a vector.

26. The method of claim 4, wherein the vector is a viral vector.

27. The method of any one of claims 1 to 5, wherein the patient is a patient with an autoimmune disease.

28. The method of claim 6, wherein the autoimmune disease is inflammatory bowel disease, rheumatoid arthritis, or pancreatitis.

29. The method of claim 7, wherein the inflammatory bowel disease is Crohn's disease or ulcerative colitis.

30. A method of preventing and/or treating sepsis, said method comprising administering an agonist of ABCF1.

31. A method of preventing and/or treating sepsis, said method comprising administering an ABCF1 protein or a polynucleotide encoding ABCF1.

32. The method of claim 10, wherein the ABCF1 protein is a soluble ABCF1 protein.

33. The method of claim 10, wherein the polynucleotide is a vector.

34. The method of claim 12, wherein the vector is a viral vector.

35. A method of treating an autoimmune disease, said method comprising administering an agonist of ABCF1.

36. A method of treating an autoimmune disease, said method comprising administering an ABCF1 protein or a polynucleotide encoding ABCF1.

37. The method of claim 15, wherein the ABCF1 protein is a soluble ABCF1 protein.

38. The method of claim 15, wherein the polynucleotide is a vector.

39. The method of claim 17, wherein the vector is a viral vector.

40. The method of any one of claims 15 to 18, wherein the autoimmune diseases is inflammatory bowel disease, arthritis, diabetes or multiple sclerosis.

41. The method of claim 19 wherein said inflammatory bowel disease is Crohn's disease or ulcerative colitis.

42. A method of determining clinical outcome of diseases and/or disorders associated with increased or decreased inflammatory and/or immune responses, said method comprising determining expression of ABCF1.

43. A method of modulating an immune response by modulating activity or expression of ABCF1.

44. A method of stimulating an immune response by inhibiting expression or activity of ABCF1.

45. The method of claim 44, wherein said immune response is an anti-cancer or anti-pathogen immune response.

46. The method of claim 45, wherein said pathogen is a viral or bacterial pathogen.

47. A method of determining ABCF1 expression comprising measuring the expression of a reporter gene under the control of the ABCF1 promoter.

48. The method of any one of claims 15 to 18, wherein the autoimmune diseases is diabetes, arthritis or multiple sclerosis.
